(19) United States
(12) Patent Application Publication
(10) Pub. No.: US 2025/0347696 A1
(43) Pub. Date: Nov. 13, 2025

(54) SELF-ACTIVATING FÖRSTER RESONANCE ENERGY TRANSFER (saFRET) BIOSENSORS AND METHODS FOR MAKING AND USING THEM

(71) Applicant: The Regents of the University of California, Oakland, CA (US)

(72) Inventors: Yingxiao WANG, San Diego, CA (US); Longwei LIU, San Diego, CA (US); Praopim LIMSAKUL, San Diego, CA (US); Shaoying LU, San Diego, CA (US)

(21) Appl. No.: 18/288,696

(22) PCT Filed: Apr. 27, 2022
(86) PCT No.: PCT/US2022/026513

§ 371 Date: Oct. 27, 2023

Related U.S. Application Data
(60) Provisional application No. 63/181,035, filed on Apr. 28, 2021.

(51) Int. Cl. G01N 33/68; C07K 14/47; C12N 9/12; C12N 15/62; C12N 15/63; G01N 33/50; G01N 33/52

(52) U.S. Cl. CPC G01N 33/68; C07K 14/47; C12N 9/12; C12N 15/62; C12N 15/63; C12Y 207/10; G01N 33/505; G01N 33/52; C07K 2319/60; C07K 2319/70

(57) ABSTRACT

In alternative embodiments, provided are self-activating Förster resonance energy transfer (saFRET) biosensors, and methods for making and using them. In alternative embodiments, provided are self-activating FRET (saFRET) biosensors, and methods that couple FRET and sequencing (FRET-Seq) to integrate random mutagenesis, fluorescence-activated cell sorting (FACS), and next-generation sequencing (NGS) to screen and identify sensitive biosensors from large-scale libraries directly in mammalian cells, utilizing the design of saFRET biosensors as provided herein.

### RELATED APPLICATIONS

This Patent Convention Treaty (PCT) International Application claims the benefit of priority under 35 U.S.C. § 119(e) of U.S. Provisional Application No. 63/181,035, filed Apr. 28, 2021. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under grants HL121365, GM125379, GM126016, EB03150, CA204704 and DK126138, awarded by the National Institutes of Health (NIH); and CBET 1344298, DMS1361421, awarded by the National Science Foundation. The government has certain rights in the invention.

### TECHNICAL FIELD

This invention generally relates to live-cell imaging, random mutagenesis, fluorescence-activated cell sorting (FACS), and next-generation sequencing (NGS). In alternative embodiments, provided are self-activating Förster resonance energy transfer (saFRET) biosensors, and methods for making and using them. In alternative embodiments, provided are self-activating FRET (saFRET) biosensors, and methods that couple FRET and sequencing (FRET-Seq) to integrate random mutagenesis, fluorescence-activated cell sorting (FACS), and next-generation sequencing (NGS) to screen and identify sensitive biosensors from large-scale libraries directly in mammalian cells, utilizing the design of saFRET biosensors as provided herein.

### BACKGROUND

Förster resonance energy transfer (FRET) biosensors are revolutionary for live-cell imaging, but their limited sensitivity has hindered broader applications.

Genetically-encoded biosensors based on FRET have revolutionized the imaging of molecular signals (for example, protein-protein interactions, protein activations, ion and small molecule dynamics) in live cells with high spatiotemporal resolution^{1,2}. However, the limited sensitivities of these biosensors have hindered their broader applications in cellular studies and drug screening^{3, 4}. At present, optimization of FRET biosensors is rather empirical and labor-intensive, often limited by the availability of accurate protein structures⁵. To address this problem, several methods have been proposed, mainly utilizing evolutionary strategies in bacteria and yeasts^{3,6,7-9}. While these methods are very well designed, they generally need additional selection steps to identify the optimized FRET biosensors since results from purified proteins or bacteria/yeasts cannot be translated directly to biosensor responses in mammalian cells. Directed evolution platforms in mammalian cells were established to evolve transcription factors and G protein-coupled receptors (GPCRs)¹⁰, or to optimize the brightness and membrane localization of the voltage reporters utilizing an elegant robotic cell picking system integrated with microscopy¹¹. Semi-rational design of relatively small-scale libraries (less than or equal to 100 variants) of FRET biosensors in mammalian cells have also been developed to improve RhoA FRET biosensors¹². However, there is no method that can systematically engineer and screen relatively large-scale libraries (for example, tens of thousands or larger) of FRET biosensors in mammalian cells for the identification of sensitive biosensors in a high throughput fashion.

Tyrosine kinases, including Fyn and ZAP70 kinases, play critical roles in various types of cell signaling and disease progression^{13,14,15}. For example, the elevated TCR signaling caused by hypermorphic R360P mutation in ZAP70, which is a key kinase for chronic lymphocytic leukemia (CLL)¹⁶, leads to clinical autoimmune phenotypes characterized by bullous pemphigoid, proteinuria, and colitis⁴. However, there is no efficient therapeutic inhibitor targeting ZAP70. The screening of kinase inhibitors has been limited mainly to conventional in vitro enzymatic assays^{17, 18}. FRET assays have high signal-to-noise ratio^{19, 20} for dynamic measurement of kinase activity in single live cells and can provide powerful tools for evaluating kinase inhibitors and their related therapeutic drugs^{21, 22}. However, despite the successful integration of FRET method with subcellular imaging in versatile screening assays for monitoring insulin-receptor activation¹⁹, SERCA2a-PLB interaction²³ and PKA kinase activity²⁴, FRET-HTDS assays have not been broadly applied for tyrosine kinase inhibitor screening, mainly due to the relatively small dynamic ranges of FRET biosensors below the robust >20% dynamic range needed for HTDS assays²⁵. The heterogeneous levels of kinase activities in individual host cells may impose additional noise and difficulty to the FRET-based screening platforms²⁶. Since some kinases such as ZAP70 are only expressed in suspension cells¹⁵, screening for kinase inhibitors can also be difficult using FRET biosensors and conventional imaging methods. Hence, a new FRET-screening design with high-sensitivity biosensors is needed to screen kinase inhibitors in a high-throughput manner.

### SUMMARY

In alternative embodiments, provided are self-activating FRET (saFRET) biosensors, and methods for using them that comprise coupling FRET and sequencing (FRET-Seq) to integrate random mutagenesis, fluorescence-activated cell sorting (FACS), and next-generation sequencing (NGS) to screen and identify sensitive biosensors from large-scale libraries directly in mammalian cells, utilizing the design of saFRET biosensors as provided herein. saFRET-based biosensors provided herein have a number of significant advantages over technologies that are based on the antibody-detection or biochemical binding assays. In the FRET technology, two fluorescent images from the donor and acceptor emissions are obtained simultaneously to calculate the ratio to represent the molecular activity. This ratiometric FRET imaging reduces the noise engendered from variations of the protein/peptide expression and concentration, the cell size and thickness, and the intensity of the excitation light source, as well as the instability of optical devices. Hence, the FRET signals can provide a much higher level of accuracy, comparing to the antibody-based or other protein-protein/peptide binding approaches.

In alternative embodiments, provided are chimeric, synthetic polypeptides comprising:
a first chimeric peptide module comprising an enhanced cyan fluorescent protein (CFP) (ECFP) domain amino terminal to a Src Homology 2 (SH2) domain or equivalent;
a second chimeric peptide module, attached to the carboxy-terminal of the SH2 domain or equivalent of the first peptide module by a peptide linker (optionally a flexible peptide linker, for example, a polyglycine, optionally between about 3 and 10, or between about 10 and 40 residues), comprising: a kinase substrate domain capable of being phosphorylated by the kinase; and, a fluorescent protein domain (optionally, a basic, constitutively fluorescent, yellow fluorescent protein-comprising domain, optionally a YPet domain or equivalent);
a third chimeric peptide module, attached to the carboxy-terminal of the YPet domain of the second peptide module by a peptide linker (optionally a flexible peptide linker), comprising a polypeptide having a kinase activity,
wherein the chimeric polypeptide acts as a self-activating Förster resonance energy transfer (saFRET) biosensor,
and the chimeric peptide has a general structure:

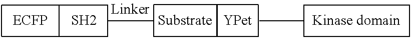

In alternative embodiments, provided are chimeric, synthetic polypeptides, wherein the polypeptide having a kinase activity is a tyrosine kinase, or a Fyn (also called Proto-oncogene tyrosine-protein kinase Fyn) or a ZAP70 (also called Zeta-chain-associated protein kinase 70) kinase, or enzymatically active fragments thereof.

In alternative embodiments, the ZAP70 kinase is a human ZAP70 kinase, optionally having the sequence:

(SEQ ID NO: 109)
MPDPAAHLPFFYGSISRAEAEEHLKLAGMADGLFLLRQCLRSLG
GYVLSLVHDVRFHHFPIERQLNGTYAIAGGKAHCGPAELCEFYS
RDPDGLPCNLRKPCNRPSGLEPQPGVFDCLRDAMVRDYVRQTWK
LEGEALEQAIISQAPQVEKLIATTAHERMPWYHSSLTREEAERK
LYSGAQTDGKFLLRPRKEQGTYALSLIYGKTVYHYLISQDKAGK
YCIPEGTKFDTLWQLVEYLKLKADGLIYCLKEACPNSSASNASG
AAAPTLPAHPSTLTHPQRRIDTLNSDGYTPEPARITSPDKPRPM
PMDTSVYESPYSDPEELKDKKLFLKRDNLLIADIELGCGNFGSV
RQGVYRMRKKQIDVAIKVLKQGTEKADTEEMMREAQIMHQLDNP
YIVRLIGVCQAEALMLVMEMAGGGPLHKFLVGKREEIPVSNVAE
LLHQVSMGMKYLEEKNFVHRDLAARNVLLVNRHYAKISDFGLSK
ALGADDSYYTARSAGKWPLKWYAPECINFRKFSSRSDVWSYGVT
MWEALSYGQKPYKKMKGPEVMAFIEQGKRMECPPECPPELYALM
SDCWIYKWEDRPDFLTVEQRMRACYYSLASKVEGPPGSTQKAEA
ACA
or
(SEQ ID NO: 110)
MPMDTSVYES PYSDPEELKD KKLFLKRDNLLIADIELGCG
NFGSVRQGVYRMRKKQIDVAIKVLKQGTEK ADTEEMMREA
QIMHQLDNPY IVRLIGVCQAEALMLVMEMA GGGPLHKFL
VGKREEIPVSN VAELLHQVSMGMKYLEEKNFVHRDLAARNV
LLVNRHYAKISDFGLSKALGADDSYYTARSAGKWPLKWYAP
ECINFRKFSSRSDVWSYGVTMWEALSYGQ KPYKKMKGPEV
MAFIEQGKRMECPPECPPELYALMSDCWIYKWEDRPDFLTV
EQRMRACYYSLASKVEGPPGSTQKAEAACA

In alternative embodiments, the Fyn kinase is a human Fyn kinase, optionally having the sequence:

(SEQ ID NO: 111)
MGCVQCKDKE ATKLTEERDG SLNQSSGYRY GTDPTPQHYPS
FGVTSIPNYNNFHAAGGQGLTVFGGVNSS SHTGTLRTRG GTG
VTLFVALYDYEARTEDDLSFHKGEKFQILNSSEGDWWEARSLTT
GETGYIPSNYVAPVDSIQAEEWYFGKLGRKDAERQLLSFGNPRG
TFLIRESETTKGAYSLSIRDWDDMKGDHVKHYKIRKLDNGGYYI
TTRAQFETLQQLVQHYSERAAGLCCRLVVPCHKGMPRLTDLSVK
TKDVWEIPRESLQLIKRLGNGQFGEVWMGTWNGNTKVAIKTLKP
GTMSPESFLEEAQIMKKLKHDKLVQLYAVVSEEPIYIVTEYMNK
GSLLDFLKDGEGRALKLPNLVDM AAQVAAGMAYIERMNYIHRD
LRSANILVGN GLICKIADFGLARLIEDNEYTARQGAKFPIKWT
APEAALYGRFTIKSDVWSFGILLTEL VTKGRVPYPGMNNREVL
EQVERGYRMPCPQD CPISLHELMI HCWKKDPEER
PTFEYLQSFL EDYFTATEPQ YQPGENL.

In alternative embodiments, the ECFP sequence comprises:

(SEQ ID NO: 115)
MVSKGEELFT GVVPILVELD GDVNGHKFSV SGEGEGDATY
GKLTLKFICT TGKLPVPWPT LVTTLTWGVQ CFSRYPDHMK
QHDFFKSAMP EGYVQERTIF FKDDGNYKTR AEVKFEGDTL
VNRIELKGID FKEDGNILGH KLEYNYISHN VYITADKQKN
GIKANFKIRH NIEDGSVQLA DHYQQNTPIG DGPVLLPDNH
YLSTQSALSK DPNEKRDHMV LLEFVTAAGI TLGMDELYK

In alternative embodiments, the SH2 domain is a human SH2, optionally having the sequence:

(SEQ ID NO: 116)
MDLPYYHGRLTKQDCETLLLKEGVDGNFLLRDSESIPGVLCLCVS
FKNIVYTYRIFREKHGYYRIQTAEGSPKQVFPSLKELISKFEKPN
QGMVVHLLKPIKRTSPSLRWRGLKLELETFVNSNSDYVDV.
or
(SEQ ID NO: 117)
MKRRSVTMTDGLTADKVTRSDGCPTSTSLPRPRDSIRSCALSMDQ
IPDLHSPMSPISESPSSPAYSTVTRVHAAPAAPSATALPASPVAR
RSSEPQLCPGSAPKTHGESDKGPHTSPSHTLGKASPSPSLSSYSD
PDSGHYCQLQPPVRGSREWAATETSSQQARSYGERLKELSENGAP
EGDWGKTFTVPIVEVTSSFNPATFQSLLIPRDNRPLEVGLLRKVK
ELLAEVDARTLARHVTKVDCLVARILGVTKEMQTLMGVRWGMELL
TLPHGRQLRLDLLERFHTMSIMLAVDILGCTGSAEERAALLHKTI
QLAAELRGTMGNMFSFAAVMGALDMAQISRLEQTWVTLRQRHTEG
AILYEKKLKPFLKSLNEGKEGPPLSNTTFPHVLPLITLLECDSAP
PEGPEPWGSTEHGVEVVLAHLEAARTVAHHGGLYHTNAEVKLQGF
QARPELLEVESTEFQMRLLWGSQGASSSQARRYEKFDKVLTALSH
KLEPAVRSSEL

In alternative embodiments, an exemplary ZAP70-saFRET biosensor as provided herein comprises the following sequence (see FIG. 3F, FIG. 5F and FIG. 5G);

(SEQ ID NO: 118)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAARMHWYFGKITRRESERLLLNPENPRGTFLVRESETTKGAYAL
SVSDFDNAKGLNVKHYKIRKLDSGGFYITSRTQFSSLQQLVAYYS
KHADGLCHRLTNVCGSTSGSGKPGSGEGSSREYACISGELELSKG
EELFTGVVPILVELDGDVNGHKFSVSGEGEGDATYGKLTLKLLCT
TGKLPVPWPTLVTTLGYGVQCFARYPDHMKQHDFFKSAMPEGYVQ
ERTIFFKDDGNYKTRAEVKFEGDTLVNRIELKGIDFKEDGNILGH
KLEYNYNSHNVYITADKQKNGIKANFKIRHNIEDGGVQLADHYQQ
NTPIGDGPVLLPDNHYLSYQSALFKDPNEKRDHMVLLEFLTAAGI
TEGMNELYKGSSAGGSAGGSAGGSAGGSAGGSGSAGGSAGGSTSA
GGSAGGSAGGSAGGSAGGSGSAGGSAGGSTSAGGSAGGSAGGSAG
GSAGGSGSAGGSAGGSTSAGGSAGGSAGGSAGGSAGGGTSRDKKL
FLKRDNLLIADIELGCGNFGSVRQGVYRMRKKQIDVAIAVLKQGT
EKADTEEMMREAQIMHQLDNPYIVRLIGVCQAEALMLVMEMAGGG
PLHKFLVGKREEIPVSNVAELLHQVSMGMKYLEEKNFVHRDLAAR
NVLLVNRHYAKISDFGLSKALGADDSYYTARSAGKWPLKWYAPEC
INFRKFSSRSDVWSYGVTMWEALSYGQKPYKKMKGPEVMAFIEQG
KRMECPPECPPELYALMSDCWIYKWEDRPDFLTVEQRMRACYYSL
ASKVEGPPGSTQKAEAACA*
(* is for a stop codon)

Domains within the exemplary ZAP70-saFRET biosensor are:

*(SEQ ID NO: 118)
(SEQ ID NO: 118)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAARMHWYFGKITRRESERLLLNPENPRGTFLVRESETTKGAYAL
SVSDFDNAKGLNVKHYKIRKLDSGGFYITSRTQFSSLQQLVAYYS
KHADGLCHRLTNVCGSTSGSGKPGSGEGSSREYACISGELELSKG
EELFTGVVPILVELDGDVNGHKFSVSGEGEGDATYGKLTLKLLCT
TGKLPVPWPTLVTTLGYGVQCFARYPDHMKQHDFFKSAMPEGYVQ
ERTIFFKDDGNYKTRAEVKFEGDTLVNRIELKGIDFKEDGNILGH
KLEYNYNSHNVYITADKQKNGIKANFKIRHNIEDGGVQLADHYQQ
NTPIGDGPVLLPDNHYLSYQSALFKDPNEKRDHMVLLEFLTAAGI
TEGMNELYKGSSAGGSAGGSAGGSAGGSAGGSGSAGGSAGGSTSA
GGSAGGSAGGSAGGSAGGSGSAGGSAGGSTSAGGSAGGSAGGSAG
GSAGGSGSAGGSAGGSTSAGGSAGGSAGGSAGGSAGGGTSRDKKL
FLKRDNLLIADIELGCGNFGSVRQGVYRMRKKQIDVAIAVLKQGT
EKADTEEMMREAQIMHQLDNPYIVRLIGVCQAEALMLVMEMAGGG
PLHKFLVGKREEIPVSNVAELLHQVSMGMKYLEEKNFVHRDLAAR
NVLLVNRHYAKISDFGLSKALGADDSYYTARSAGKWPLKWYAPEC
INFRKFSSRSDVWSYGVTMWEALSYGQKPYKKMKGPEVMAFIEQG
KRMECPPECPPELYALMSDCWIYKWEDRPDFLTVEQRMRACYYSL
ASKVEGPPGSTOKAEAACA*

The above is the ZAP70-saFRET biosensor amino acid sequence, related to FIG. 3F, 5F, 5G.
Note for the exemplary ZAP70-saFRET biosensor:
The underlined amino acid is the ZAP70 kinase domain sequence, or

(SEQ ID NO: 120)
DKKLFLKRDNLLIADIELGCGNFGSVRQGVYRMRKKQIDVAIAVL
KQGTEKADTEEMMREAQIMHQLDNPYIVRLIGVCQAEALMLVMEM
AGGGPLHKFLVGKREEIPVSNVAELLHQVSMGMKYLEEKNFVHRD
LAARNVLLVNRHYAKISDFGLSKALGADDSYYTARSAGKWPLKWY
APECINFRKFSSRSDVWSYGVTMWEALSYGQKPYKKMKGPEVMAF
IEQGKRMECPPECPPELYALMSDCWIYKWEDRPDFLTVEQRMRAC
YYSLASKVEGPPGSTQKAEAACA

The (red labeled) sequence is the EV linker, or

(SEQ ID NO: 121)
GSSAGGSAGGSAGGSAGGSAGGSGSAGGSAGGSTSAGGSAGGSAG
GSAGGSAGGSGSAGGSAGGSTSAGGSAGGSAGGSAGGSAGGSGSA
GGSAGGSTSAGGSAGGSAGGSAGGSAGGGTSR

The cyan-labeled (cyan labeled) amino acid represents the ECFP sequence, or

(SEQ ID NO: 122)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAA

and the yellow labeled (yellow labeled) part represents the YPet sequence, or

(SEQ ID NO: 123)
SKGEELFTGVVPILVELDGDVNGHKFSVSGEGEGDATYGKLTLKL
LCTTGKLPVPWPTLVTTLGYGVQCFARYPDHMKQHDFFKSAMPEG
YVQERTIFFKDDGNYKTRAEVKFEGDTLVNRIELKGIDFKEDGNI
LGHKLEYNYNSHNVYITADKQKNGIKANFKIRHNIEDGGVQLADH
YQQNTPIGDGPVLLPDNHYLSYQSALFKDPNEKRDHMVLLEFLTA
AGITEGMNELYK

The pink labeled (pink labeled) amino acid represents part of the substrate sequence, or SREYACI (SEQ ID NO:44), which in alternative embodiments is replaced with alternative substrates sequence as described herein, for example, alternative substrates are listed in FIG. 3G and FIG. 3H.

In alternative embodiments, the EV linker is changed to other commonly used linkers to construct a new saFRET biosensor, e.g., P2A linker, 34-mer linker, 17-mer linker, see below for exemplary sequences.

In alternative embodiments, an exemplary Fyn-saFRET biosensor as provided herein comprises the following sequence (see FIG. 2C, FIG. 2D and FIG. 2E);

(SEQ ID NO: 119)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAARMHWYFGKITRRESERLLLNPENPRGTFLVRESETTKGAYAL
SVSDFDNAKGLNVKHYKIRKLDSGGFYITSRTQFSSLQQLVAYYS
KHADGLCHRLTNVCGSTSGSGKPGSGEGSEKIEGTYHWFELSKGE
ELFTGVVPILVELDGDVNGHKFSVSGEGEGDATYGKLTLKLLCTT
GKLPVPWPTLVTTLGYGVQCFARYPDHMKQHDFFKSAMPEGYVQE
RTIFFKDDGNYKTRAEVKFEGDTLVNRIELKGIDFKEDGNILGHK
LEYNYNSHNVYITADKQKNGIKANFKIRHNIEDGGVQLADHYQQN
TPIGDGPVLLPDNHYLSYQSALFKDPNEKRDHMVLLEFLTAAGIT
EGMNELYKGSSAGGSAGGSAGGSAGGSAGGSGSAGGSAGGSTSAG
GSAGGSAGGSAGGSAGGSGSAGGSAGGSTSAGGSAGGSAGGSAGG
SAGGSGSAGGSAGGSTSAGGSAGGSAGGSAGGSAGGGTSRKDVWE
IPRESLQLIKRLGNGQFGEVWMGTWNGNTKVAIKTLKPGTMSPES
FLEEAQIMKKLKHDKLVQLYAVVSEEPIYIVTEYMNKGSLLDFLK
DGEGRALKLPNLVDMAAQVAAGMAYIERMNYIHRDLRSANILVGN
GLICKIADFGLARLIEDNEYTARQGAKFPIKWTAPEAALYGRFTI
KSDVWSFGILLTELVTKGRVPYPGMNNREVLEQVERGYRMPCPQD
CPISLHELMIHCWKKDPEERPTFEYLQSFLEDYFTATEPQYQP
GENL*
(* is for a stop codon)

Domains within the exemplary Fyn-saFRET biosensor:

(SEQ ID NO: 119)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAARMHWYFGKITRRESERLLLNPENPRGTFLVRESETTKGAYAL
SVSDFDNAKGLNVKHYKIRKLDSGGFYITSRTQFSSLQQLVAYYS
KHADGLCHRLTNVCGSTSGSGKPGSGEGSEKIEGTYHWFELSKGE
ELFTGVVPILVELDGDVNGHKFSVSGEGEGDATYGKLTLKLLCTT
GKLPVPWPTLVTTLGYGVQCFARYPDHMKQHDFFKSAMPEGYVQE
RTIFFKDDGNYKTRAEVKFEGDTLVNRIELKGIDFKEDGNILGHK
LEYNYNSHNVYITADKQKNGIKANFKIRHNIEDGGVQLADHYQQN
TPIGDGPVLLPDNHYLSYQSALFKDPNEKRDHMVLLEFLTAAGIT
EGMNELYKGSSAGGSAGGSAGGSAGGSAGGSGSAGGSAGGSTSAG
GSAGGSAGGSAGGSAGGSGSAGGSAGGSTSAGGSAGGSAGGSAGG
SAGGSGSAGGSAGGSTSAGGSAGGSAGGSAGGSAGGGTSRKDVWE
IPRESLQLIKRLGNGQFGEVWMGTWNGNTKVAIKTLKPGTMSPES
FLEEAQIMKKLKHDKLVQLYAVVSEEPIYIVTEYMNKGSLLDFLK
DGEGRALKLPNLVDMAAQVAAGMAYIERMNYIHRDLRSANILVGN
GLICKIADFGLARLIEDNEYTARQGAKFPIKWTAPEAALYGRFTI
KSDVWSFGILLTELVTKGRVPYPGMNNREVLEQVERGYRMPCPQD
CPISLHELMIHCWKKDPEERPTFEYLOSFLEDYFTATEPQYQPGE
NL*
*Means stop codon.

The above exemplary Fyn-saFRET biosensor amino acid sequence is related to FIG. 2C, 2D, 2E.
Notes for the exemplary Fyn-saFRET biosensor:
The underlined amino acid is the Fyn kinase domain sequence, or

(SEQ ID NO: 124)
KDVWEIPRESLQLIKRLGNGQFGEVWMGTWNGNTKVAIKTLKPGT
MSPESFLEEAQIMKKLKHDKLVOLYAVVSEEPIYIVTEYMNKGSL
LDFLKDGEGRALKLPNLVDMAAQVAAGMAYIERMNYIHRDLRSAN
ILVGNGLICKIADFGLARLIEDNEYTARQGAKFPIKWTAPEAALY
GRFTIKSDVWSFGILLTELVTKGRVPYPGMNNREVLEQVERGYRM
PCPQDCPISLHELMIHCWKKDPEERPTFEYLQSFLEDYFTATEPQ
YQPGENL

The red labeled (red labeled) part is the EV linker, or

(SEQ ID NO: 125)
GSSAGGSAGGSAGGSAGGSAGGSGSAGGSAGGSTSAGGSAGGSAG
GSAGGSAGGSGSAGGSAGGSTSAGGSAGGSAGGSAGGSAGGSGSA
GGSAGGSTSAGGSAGGSAGGSAGGSAGGGTSR

The cyan-labeled (cyan labeled) amino acid represents the ECFP sequence, or

(SEQ ID NO: 126)
MVSKGEELFTGVVPILVELDGDVNGHRFSVSGEGEGDATYGKLTL
KFICTTGKLPVPWPTLVTTLTWGVQCFSRYPDHMKQHDFFKSAMP
EGYVQERTIFFKDDGNYKTRAEVKFEGDTLVNRIELKGIDFKEDG
NILGHKLEYNYISHNVYITADKQKNGIKANFKIRHNIEDGSVQLA
DHYQQNTPIGDGPVLLPDNHYLSTQSALSKDPKEKRDHMVLLEFV
TAA

The yellow labeled (yellow labeled) part represents the YPet sequence, or

(SEQ ID NO: 127)
SKGEELFTGVVPILVELDGDVNGHKFSVSGEGEGDATYGKLTLKL
LCTTGKLPVPWPTLVTTLGYGVQCFARYPDHMKQHDFFKSAMPEG
YVQERTIFFKDDGNYKTRAEVKFEGDTLVNRIELKGIDFKEDGNI
LGHKLEYNYNSHNVYITADKQKNGIKANFKIRHNIEDGGVQLADH
YQQNTPIGDGPVLLPDNHYLSYQSALFKDPNEKRDHMVLLEFLTA
AGITEGMNELYK

The pink labeled (pink labeled) amino acid, or EGTYHWF (SEQ ID NO:3), represents part of the substrate sequence, which in alternative embodiments is replaced by alternative substrate sequences as provided herein, for example, as listed in FIG. 2D and FIG. 2E.

In alternative embodiments, the EV linker is changed to alternative linkers known in the art to construct an alternative saFRET biosensor, for example, using: a P2A linker, 34-mer linker, 17-mer linker, optionally having the sequences:

P2A linker:
(SEQ ID NO: 128)
GSGATNFSLLKQAGDVEENPGP
T2A linker:
(SEQ ID NO: 129)
GSGEGRGSLLTCGDVEENPGP
34 mer linker:
(SEQ ID NO: 130)
GSTSGSGKPGSGEGSTKGSTSGSGKPGSGEGSTK
17 mer linker:
(SEQ ID NO: 131)
GSTSGSGKPGSGEGSTK

In alternative embodiments, provided are nucleic acids encoding the chimeric, synthetic polypeptide as provided herein.

In alternative embodiments, provided are expression vectors (optionally vectors, plasmids, phages, phagemids or recombinant viruses) comprising or having contained therein a nucleic acid as provided herein.

In alternative embodiments, provided are cells comprising or having contained therein a nucleic acid as provided herein, or a chimeric, synthetic polypeptide as provided herein, wherein optionally the cell is a human cell, and optionally the human cell is a lymphocyte or a T cell, or a CAR T cell.

In alternative embodiments, provided are methods for identifying a kinase inhibitor in a cell, comprising:
(a) providing a cell expressing a nucleic acid as provided herein, and expressing a chimeric, synthetic polypeptide as provided herein; or, providing a cell comprising or having contained therein a chimeric, synthetic polypeptide as provided herein,
wherein optionally the cell is a human cell, and optionally the human cell is a lymphocyte or a T cell, or a CAR T cell;
(b) providing a test molecule, wherein optionally the test molecule is a synthetic molecule or a molecule from a kinase inhibitor library;
(c) contacting the test molecule with the cell and measuring or detecting a change in a detectable signal generated by the chimeric, synthetic polypeptide as provided herein, as compared to a cell expressing the chimeric, synthetic polypeptide as provided herein, that has not been contacted with the test molecule, wherein a change in detectable signal identifies the test molecule as an inhibitor of the polypeptide having the kinase activity in the third chimeric peptide module.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-E illustrate construction and validation of exemplary self-activating FRET (saFRET) biosensors, as provided herein:

FIG. 1A schematically illustrates mammalian cell biosensor library development, screening and sequencing in mammalian cells;

FIG. 1B schematically illustrates domain structure and activation mechanism of an exemplary saFRET biosensor with a fused kinase domain;

FIG. 1C-E illustrate replacement of the kinase domain by its kinase-dead version abolished the FRET ratio and the Fyn inhibitor PP1-induced dynamic changes of the saFRET biosensor during live-cell imaging,

FIG. 1C-D illustrate representative images (FIG. 1C) and time courses (FIG. 1D) of Fyn-saFRET biosensor with active kinase domain (KA) or kinase-dead domain (KD) before and after PP1 treatment;

FIG. 1E graphically illustrates measuring basal FRET ratios in active kinase domain and kinase-dead domain (KD);

FIG. 1F illustrates an image of a gel showing biosensor phosphorylation; and

FIG. 1G schematically illustrates an exemplary modular design of a sensing unit, this functional Fyn-saFRET biosensor was utilized to create a template for the library generation, as discussed in detail in Example 1, below.

FIG. 2A-F illustrate identification of biosensors by NGS and sequence-function analysis:

FIG. 2A illustrates an exemplary workflow of sequencing data analysis;

FIG. 2B graphically illustrates four-dimensional (4D) plot of the enrichment ratios (Eᵥ) of substrate sequences from different sorting groups;

FIG. 2A illustrates representative time-lapse images of the parental (WT) and improved biosensor (EKIEGTYHWF) (SEQ ID NO:1) before and after PP1 treatment;

FIG. 2D graphically illustrates the quantified dynamic changes of biosensor variants (EKIEGTYXXX) (SEQ ID NO:2) upon PP1 treatment, including the biosensor variants EGTYHWF (SEQ ID NO:3); EGTYIHY (SEQ ID NO:4); EGTYIWC (SEQ ID NO:5); EGTYFQC (SEQ ID NO:6); EGTYHQM (SEQ ID NO: 7); EGTYYFF (SEQ ID NO:8); EGTYIHW (SEQ ID NO:9); EGTYFMC (SEQ ID NO: 10); EGTYMYT (SEQ ID NO:11); EGTYFHF (SEQ ID NO:12); EGTYMWE (SEQ ID NO:13); EGTYTFA (SEQ ID NO:14); EGTYWCH (SEQ ID NO:15); EGTYYIF (SEQ ID NO:16); EGTYCNF (SEQ ID NO:17); EGTYPCQ (SEQ ID NO: 18); EGTYDPQ (SEQ ID NO:19); EGTYIIY (SEQ ID NO:20); EGTYVLW (SEQ ID NO:21); EGTYCFM (SEQ ID NO:22); EGTYNHM (SEQ ID NO:23); EGTYFEY (SEQ ID NO:24); EGTYEAF (SEQ ID NO:25); EGTYLLL (SEQ ID NO: 26); EGTYPFT (SEQ ID NO:27); EGTYHIL (SEQ ID NO:28); EGTYIDI (SEQ ID NO: 29); EGTYINF (SEQ ID NO:30); EGTYVGI (SEQ ID NO:31); EGTYDFE (SEQ ID NO:32); EGTYPLM (SEQ ID NO:33); EGTYVFM (SEQ ID NO:34); EGTYVFW (SEQ ID NO:35); EGTYVQF (SEQ ID NO:36); EGTYIDF (SEQ ID NO: 37); EGTYVEF (SEQ ID NO:38); EGTYRGA (SEQ ID NO:39); EGTYWFM (SEQ ID NO:40); EGTYVRL (SEQ ID NO:41);

FIG. 2E graphically illustrates time courses of normalized ECFP/FRET ratio of the biosensor variants (see FIG. 2D for biosensor variant SEQ IDs), with that of the parental biosensor labeled in black; and

FIG. 2F graphically illustrates normalized FRET ratios as a function of time, as discussed in detail in Example 1, below.

FIG. 3A-H illustrate development and optimization of an exemplary ZAP70 FRET biosensor:

FIG. 3A schematically illustrates the design of an exemplary self-activating ZAP70 FRET biosensor as the screening template, with as substrates XXXYVNVSGEL (SEQ ID NO:42) or SREYXXXSGEL (SEQ ID NO:43);

FIG. 3B-C illustrates representative images (FIG. 3B) and graphically illustrate time courses (FIG. 3C) of FRET ratios of an exemplary ZAP70 saFRET biosensor with Active- or Dead-kinase domain, before and after TAK-659 treatment;

FIG. 3D graphically illustrates a 4D plot of the four enrichment ratios (Eᵥ) of substrate sequences, and the enrichment ratios in KAH group (Eᵥ(KAH)) was color-coded, whereas Ev(KAL), Ev(KDH) and Ev(KDL) are plotted along the three dimensional coordinates, and the selected substrate sequences are highlighted with colors represented by the values of their Ev(KAH);

FIG. 3E graphically illustrates a scatter plot of the substrates: exemplary ZAP70 saFRET biosensors with the top 10 highest products of Ev(KAH) and Ev(KDL) were labeled in red, or the ▴ (better biosensors) or blue or the • (worse biosensors) SREYACI (SEQ ID NO:44), SREYYDM (SEQ ID NO:45), SREYSEI (SEQ ID NO:46); SREYEKM (SEQ ID NO:47), SREYAFP (SEQ ID NO:48);

FIG. 3F illustrates time-lapse images of the parental (WT) and two selected saFRET biosensors after TAK-659 treatment;

FIG. 3G graphically illustrates percentage changes of saFRET biosensor variants after TAK-659 treatment, where time courses of FRET ratio of the selected saFRET biosensor variants (SREYXXXSGEL (SEQ ID NO:43)), with that of the parental biosensor (WT) marked in black, the color bar indicates ECFP/FRET ratio, with hot and cold colors representing the high and low ratios, respectively, the selected saFRET biosensor variants are SREYACI (SEQ ID NO:44), SREYYDM (SEQ ID NO:45), SREYSEI (SEQ ID NO:46); SREYEKM (SEQ ID NO: 47), SREYAFP (SEQ ID NO:48); SREYEYC (SEQ ID NO:49), SREYEYM (SEQ ID NO:50), SREYYYP (SEQ ID NO:51), SREYEQM (SEQ ID NO:52); and

FIG. 3H graphically illustrates normalized FRET ratios of selected saFRET biosensor variants (see FIG. 3G) as a function of time, as discussed in detail in Example 1, below.

FIG. 4A-J illustrate data showing the sensitivity and specificity of an exemplary ZAP70 FRET biosensor in a human T cell:

FIG. 4A schematically illustrates a working mechanism of the exemplary ZAP70 biosensor in reporting TCR signaling;

FIG. 4B-E illustrate time-lapse ECFP/FRET ratio (FRET ratio) images (FIG. 4B, FIG. 4D) and time courses (FIG. 4D, FIG. 4E) of improved (FIG. 4B, FIG. 4C) or parental (WT) (FIG. 4E, FIG. 4E) biosensors before and after TCR activation induced by CD3/CD28 antibody stimulation,
where FIG. 4C illustrates use of biosensor SREYACI (SEQ ID NO:44), and FIG. 4E illustrates use of biosensor SREYVNV (SEQ ID NO:53);

FIG. 4F schematically illustrates a schematic of membrane-bound biosensors which target different membrane compartments, where Lyn- and Kras-ZAP70 biosensors target the lipid rafts or non-raft regions, respectively;

FIG. 4G illustrates time-lapse FRET ratio images of ZAP70 activities in different membrane compartments after TCR activation;

FIG. 4H graphically illustrates time courses of ECFP/FRET ratio of an exemplary ZAP70 biosensor in different membrane compartments before and after CD3/CD28 antibody stimulation;

FIG. 4I graphically illustrates a schematic of CD19-CAR Jurkat T cell engaging with a CD19⁺ tumor Toledo cell;

FIG. 4J graphically illustrates time-lapse FRET ratio images of CAR-T cell expressing the improved exemplary ZAP70 biosensor before and after the engagement with a target tumor Toledo cell,
as discussed in detail in Example 1, below.

FIG. 5A-G illustrates high-throughput drug screening platform using an exemplary saFRET biosensor:

FIG. 5A illustrates a schematic of the high throughput drug screening platform;

FIG. 5B illustrates FRET-Ratio images of the cells with different inhibitors;

FIG. 5C graphically illustrates a summary of screening results;

FIG. 5C graphically illustrates the activity of top 10 selected inhibitors;

FIG. 5E graphically illustrates counter screening using a mutant biosensor with a kinase-dead domain to subtract the noise engendered from non-specific fluorescence, where the Scatter plot illustrates the FRET ratio changes in the positive and negative screenings using an exemplary saFRET biosensor fused with an active kinase or a kinase-dead domain, respectively;

FIG. 5F illustrates FRET ratio images of live-cell imaging with different inhibitors;

FIG. 5G graphically illustrates time courses of the FRET ratio before and after inhibitor treatment,
as discussed in detail in Example 1, below.

FIG. 6A-L illustrate data showing inhibition of T cell activation by the HTDS-identified ZAP70 inhibitors Staurosporine and AZD7762:

FIG. 6A schematically illustrates an exemplary experimental scheme and timeline for experiments in FIG. 6B-D, where the Jurkat T cells were pre-treated with inhibitors for 30 minutes before anti-TCR stimulation by anti-CD3/CD28 antibodies for 5 minutes;

FIG. 6B illustrates immunostaining images of pLAT (Y191) in Jurkat T cells with different inhibitor pre-treatments;

FIG. 6C graphically illustrates quantification of pLAT (Y191) intensity of single cells in different groups;

FIG. 6D graphically illustrates quantification of pZAP70 (Y493) intensity of single cells in different groups;

FIG. 6E schematically illustrates an exemplary experimental scheme and timeline for CD69 staining experiment;

FIG. 6F graphically illustrates a flow-cytometry analysis of CD69 expression in T cells after anti-TCR stimulation, with different inhibitor pre-treatments.

FIG. 6G schematically illustrates an exemplary experimental scheme and timeline of P116 cells reconstituted with ZAP70;

FIG. 6H graphically illustrates CD69 expression in P116 cells with or without the expression of ZAP70 (WT) and its mutant (R360P);

FIG. 6I graphically illustrates quantification of pZAP70 (Y493) intensity of single cells in different P116 groups;

FIG. 6J illustrates images of pLAT (Y191) in P116-ZAP70 R360P cells with different inhibitor pre-treatments;

FIG. 6K graphically illustrates quantification of pLAT (Y191) intensity of single cells in P116-ZAP70 R360P cells with different inhibitor pre-treatment. (n>150 for each group; and

FIG. 6L graphically illustrates quantification flow-cytometric analysis of CD69 expression in P116-ZAP70-R360P cells with different inhibitor pre-treatment, where ZAP70-WT or ZAP70-R360P expression levels were indicated by YPet intensity,
as discussed in detail in Example 1, below.

FIG. 7A-D illustrates mammalian cell library screening by FACS;

FIG. 7A graphically illustrates Sanger sequencing results showing random mutagenesis in the mutation region of the substrate peptide where EKIXXXYGVV (SEQ ID NO:54) represents library 1 (Lib1) with active (KA) or dead kinase (KD), and EKIEGTYXXX (SEQ ID NO:2) represents library 2 (Lib2) with active (KA) or dead kinase (KD);

FIG. 7B schematically illustrates an exemplary mammalian cell library screening by FACS, the ECFP/FRET ratio of the FRET biosensor variants expressed in single cells was analyzed;

FIG. 7C schematically and graphically illustrates different control groups in FACS experiment, from left to right: only ECFP-expressing cells, only YPet-expressing cells, co-expression of ECFP- and YPet-expressing cells, mixture of only ECFP- or YPet-expressing cells, cells with KD FRET biosensor, cells with KA FRET biosensor, and the top panel shows the relation between YPet intensity (y-axis) and ECFP intensity (x-axis), and the bottom panel shows the relation between FRET intensity (y-axis) and ECFP intensity (x-axis);

FIG. 7D graphically illustrates data from a FACS experiment: after gate setting using the control biosensors in FIG. 7C, we analyzed and sorted the cells from different libraries, and after single-cell gating, the cells with medium expression of FRET biosensor (as represented by YPet expression intensity) were gated and divided into High and Low ECFP/FRET ratio groups, and based on the ECFP/FRET ratio shown in the histogram plot, we can successfully separate the cells with different ratios (CFP/FRET),
as discussed in detail in Example 1, below.

FIG. 8A-D illustrates the positive correlation of biosensors between the improved performance and the product of Eᵥ(KAH) and Eᵥ(KDL):

FIG. 8A graphically illustrates how desired biosensors identified were verified to be not enriched in either KAL or KDH group;

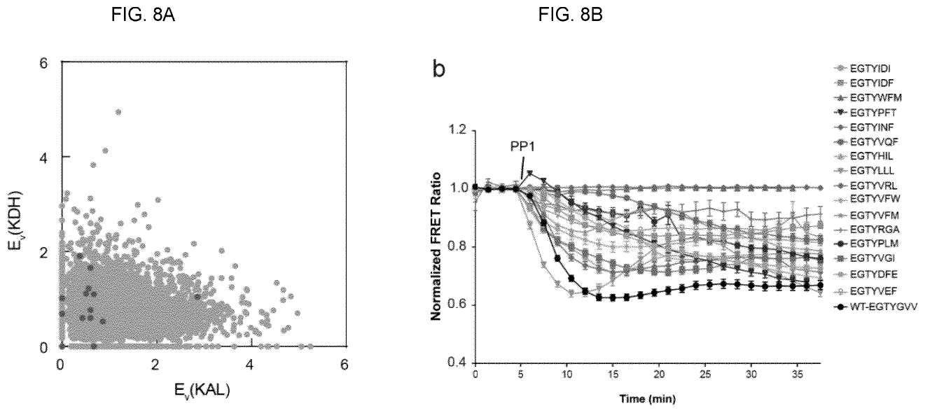

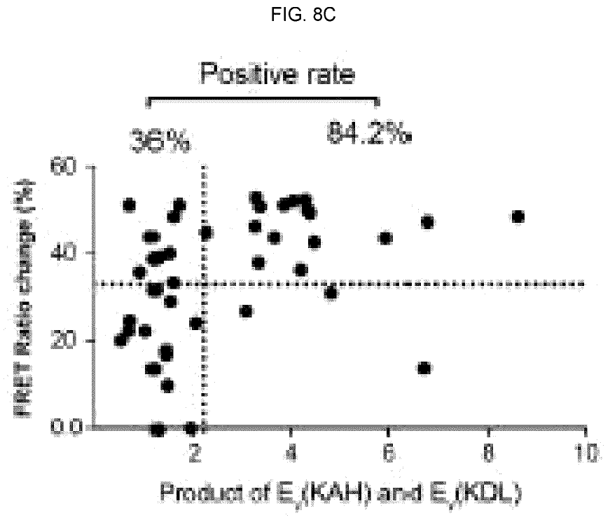

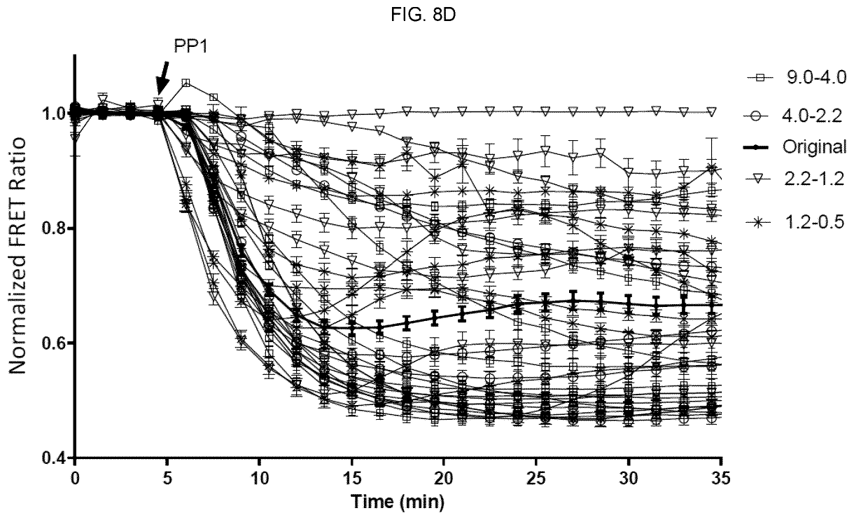

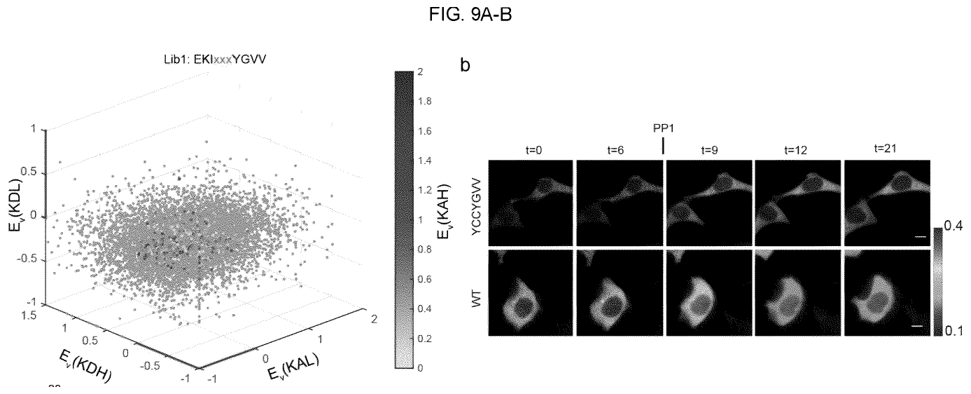

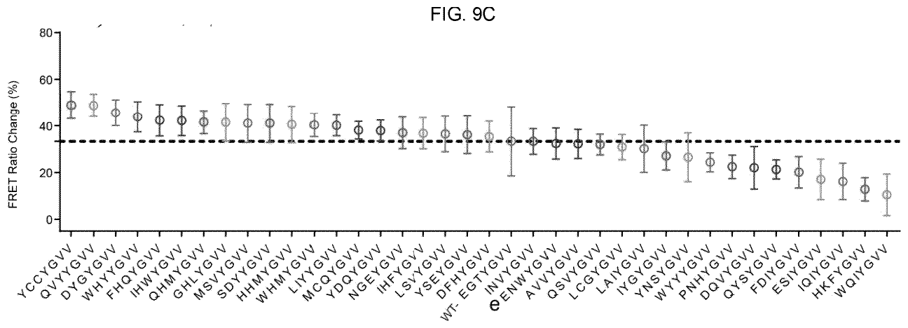

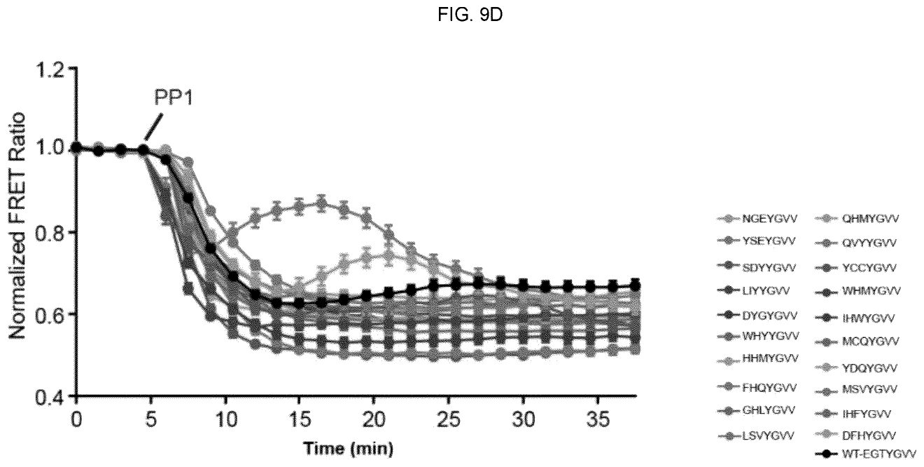

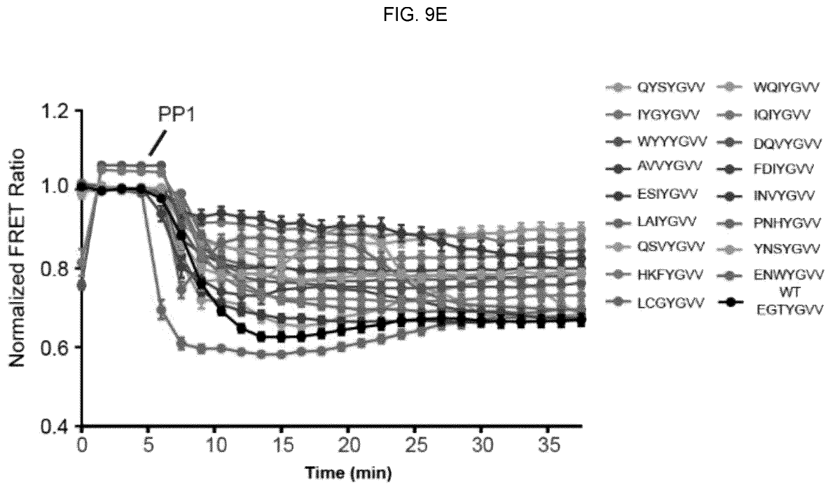

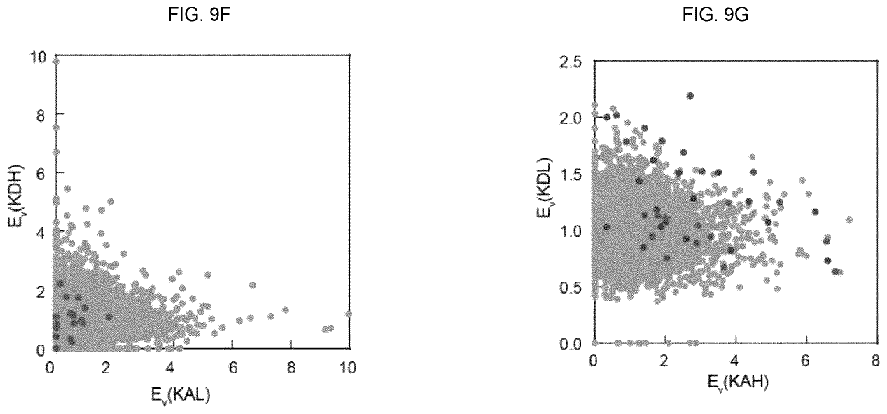

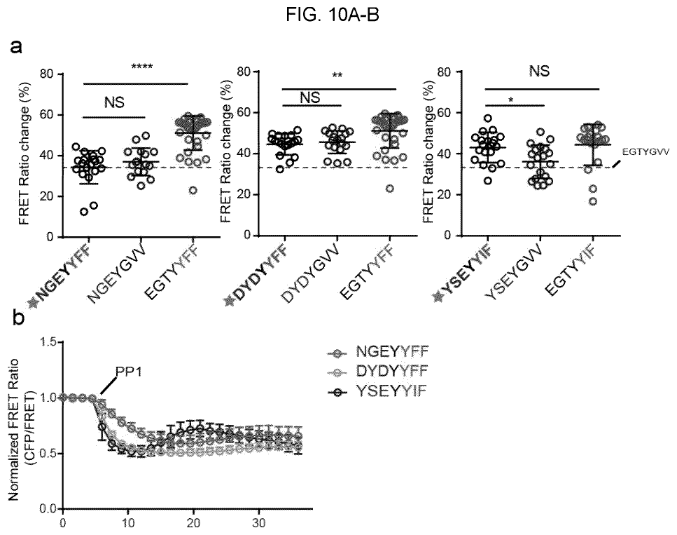

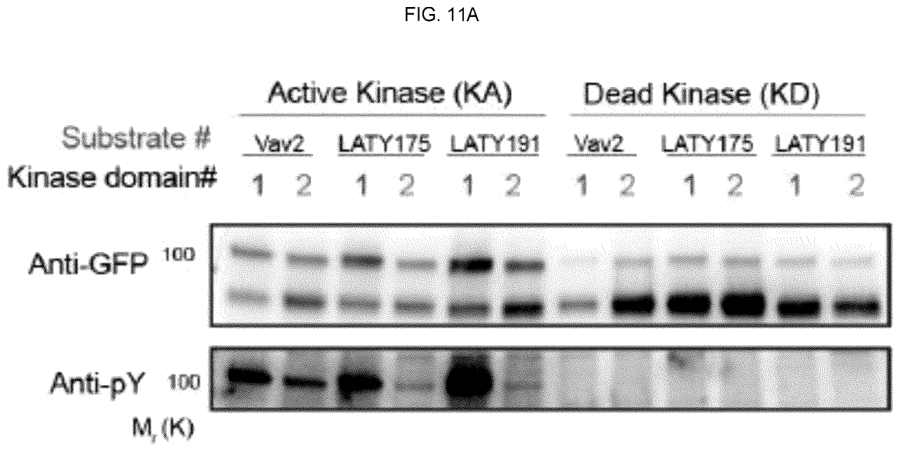

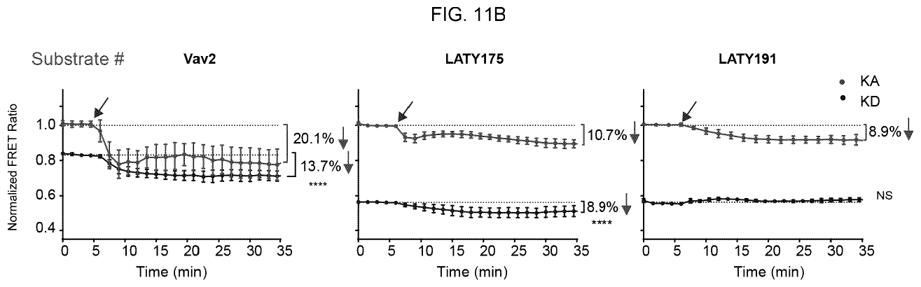

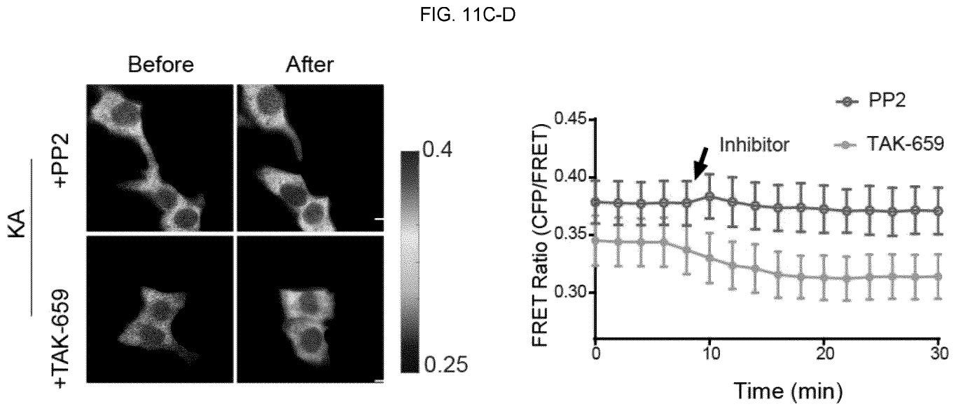

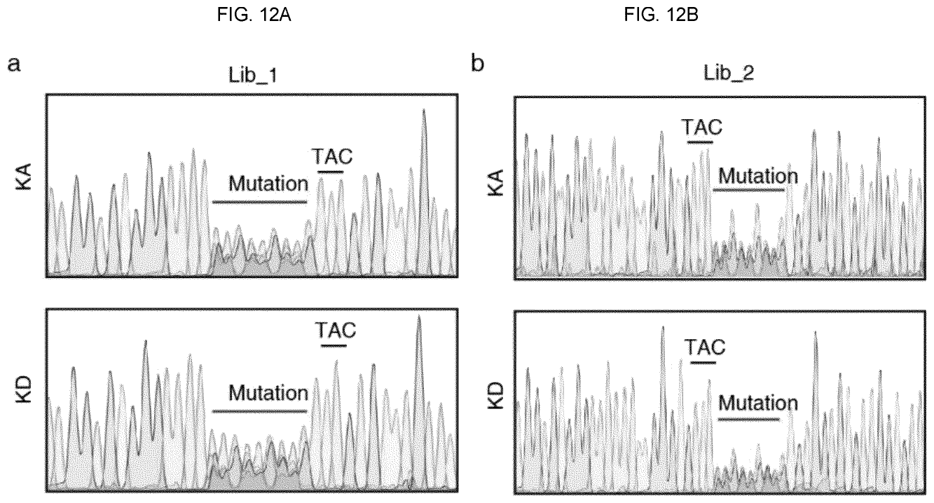

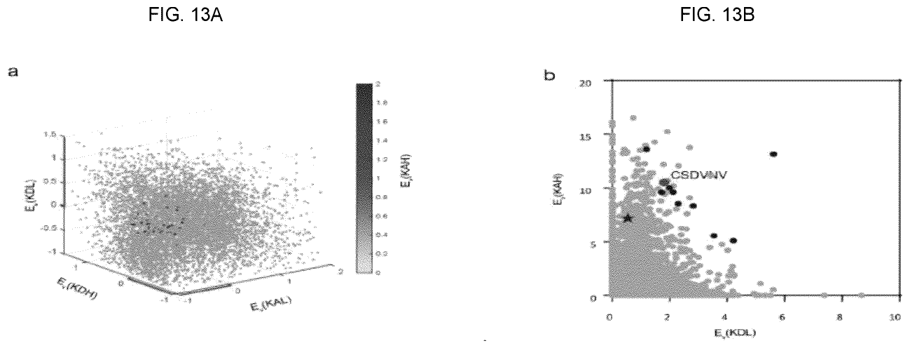

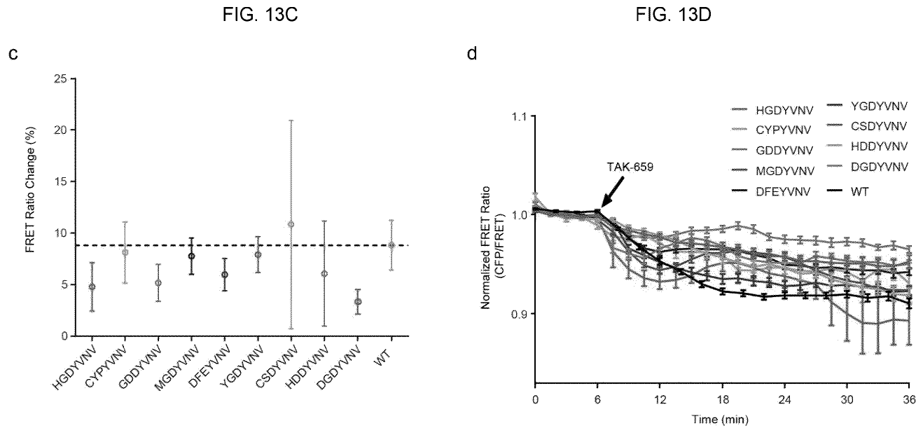

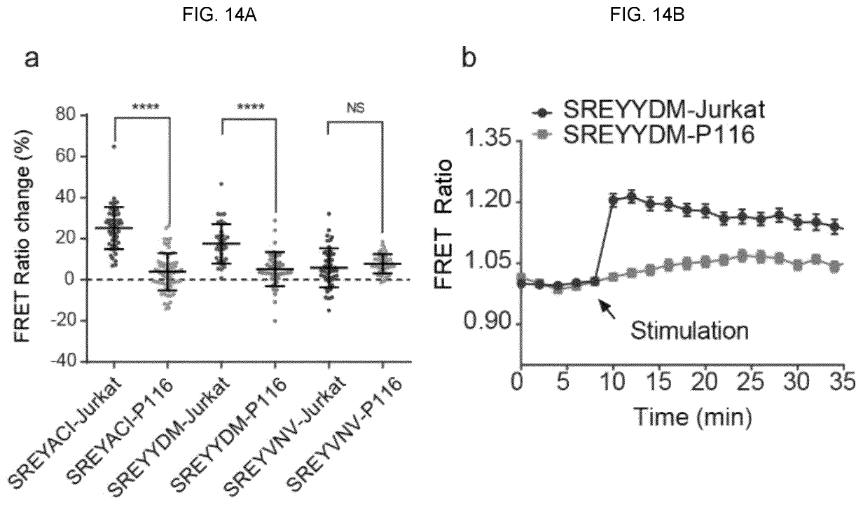

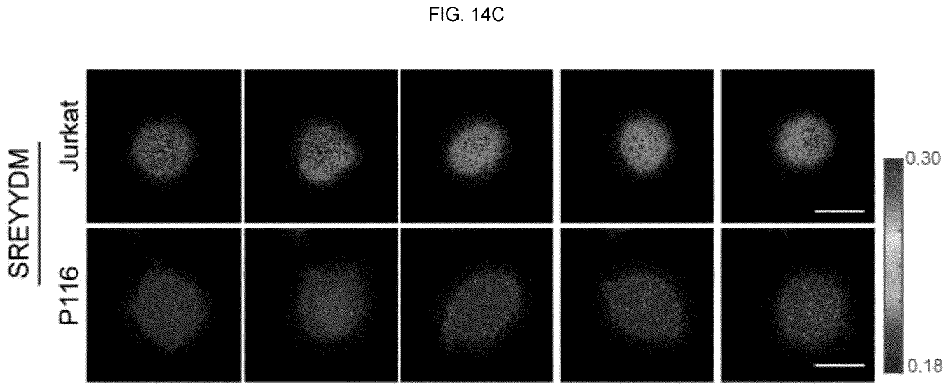

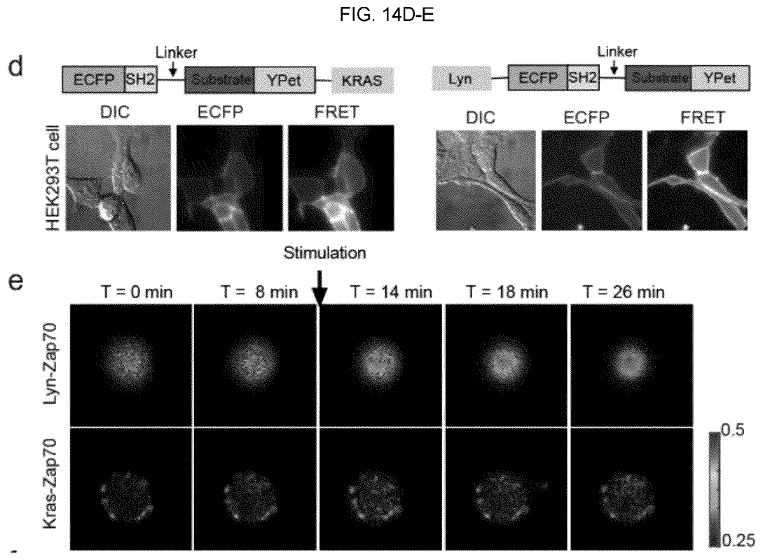

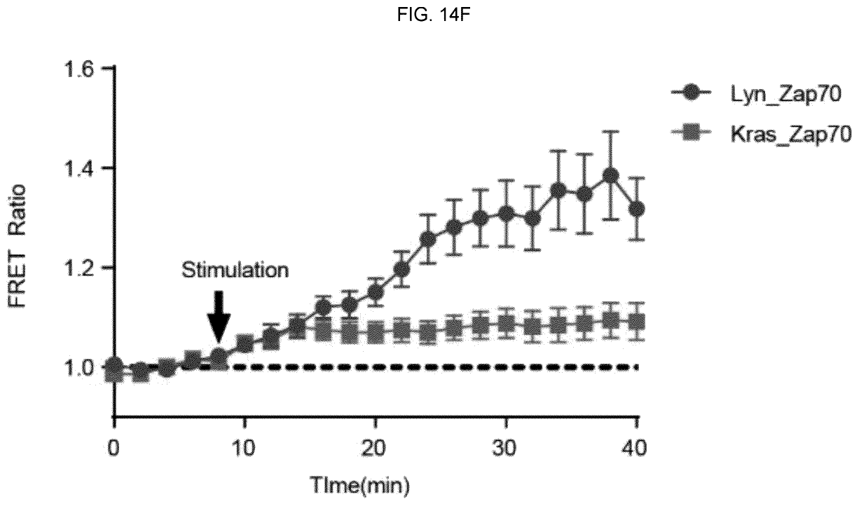

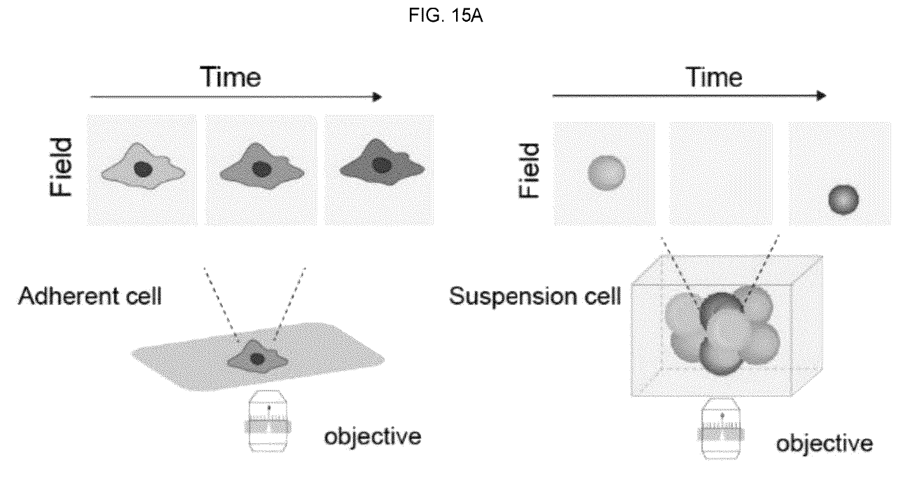

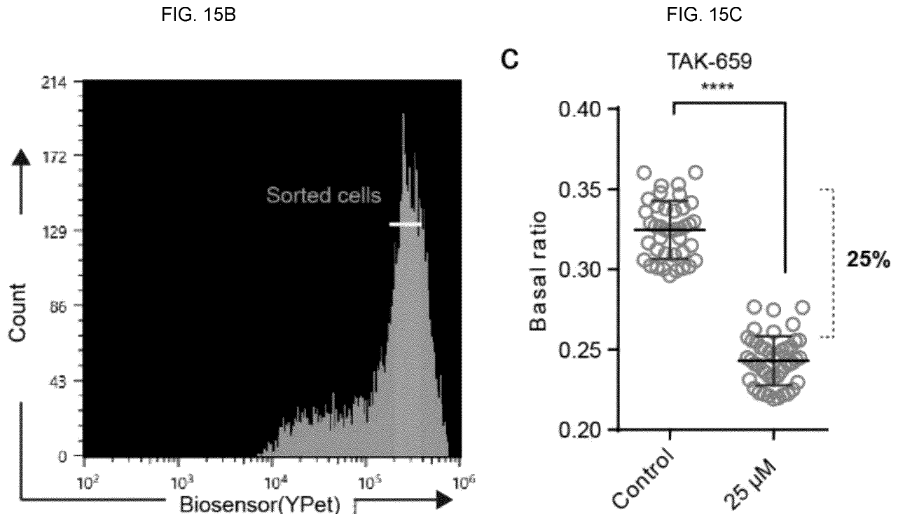

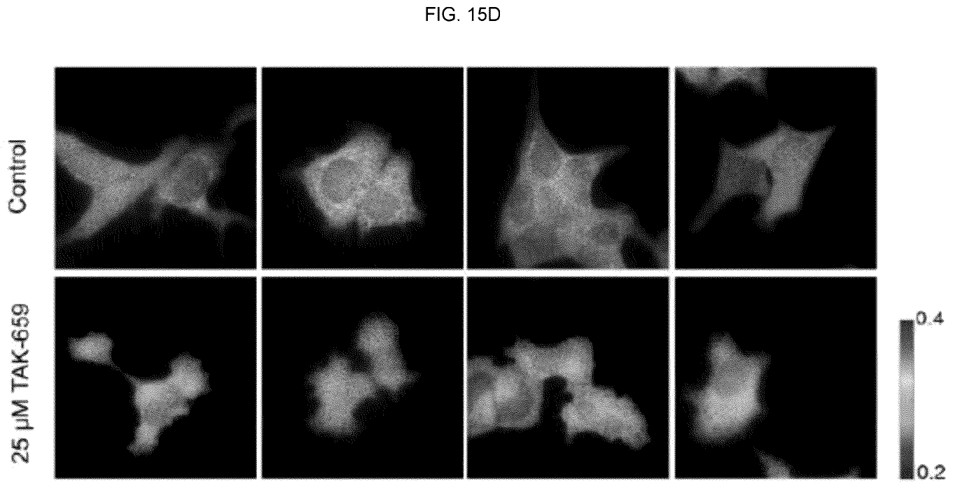

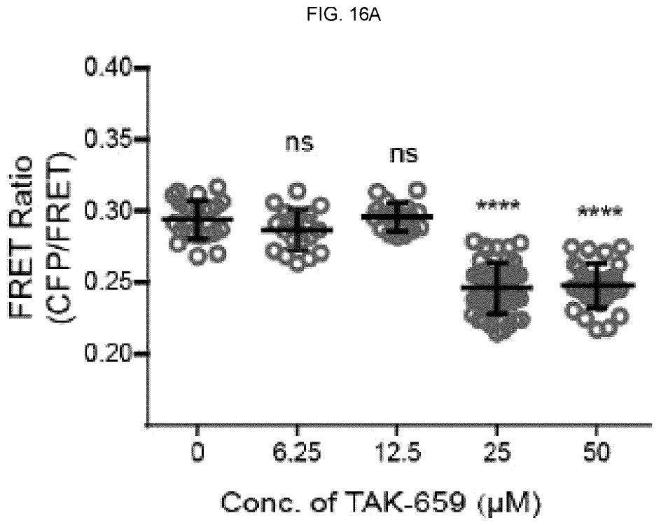

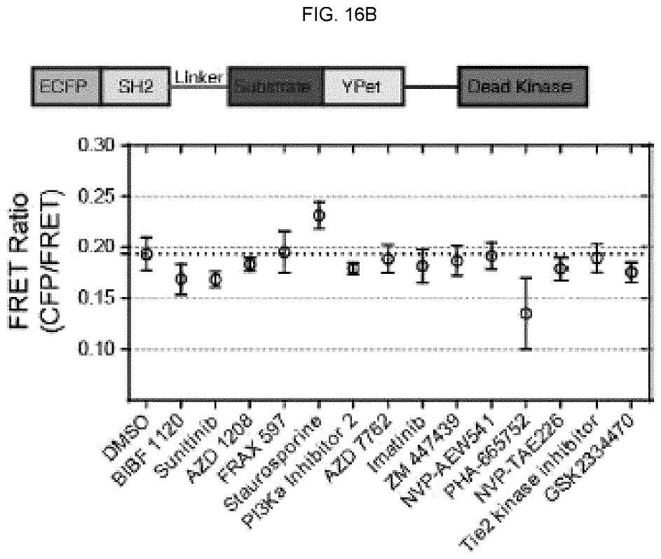

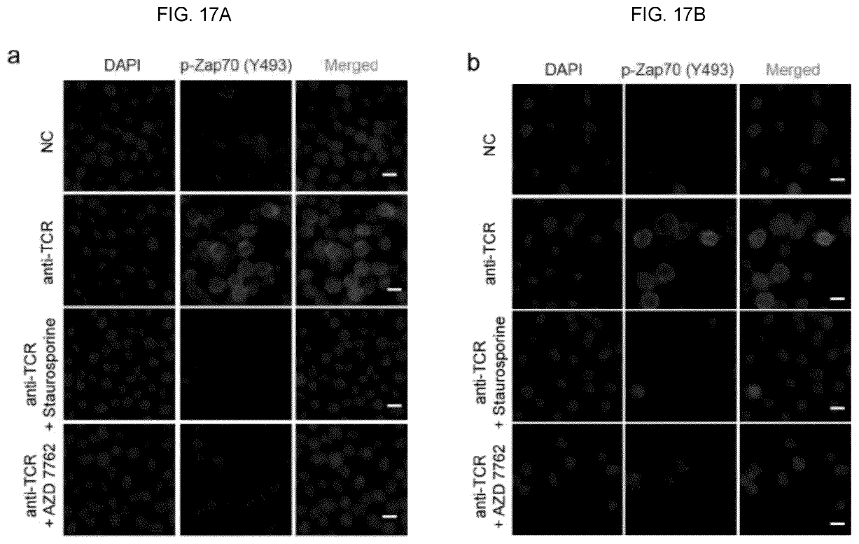

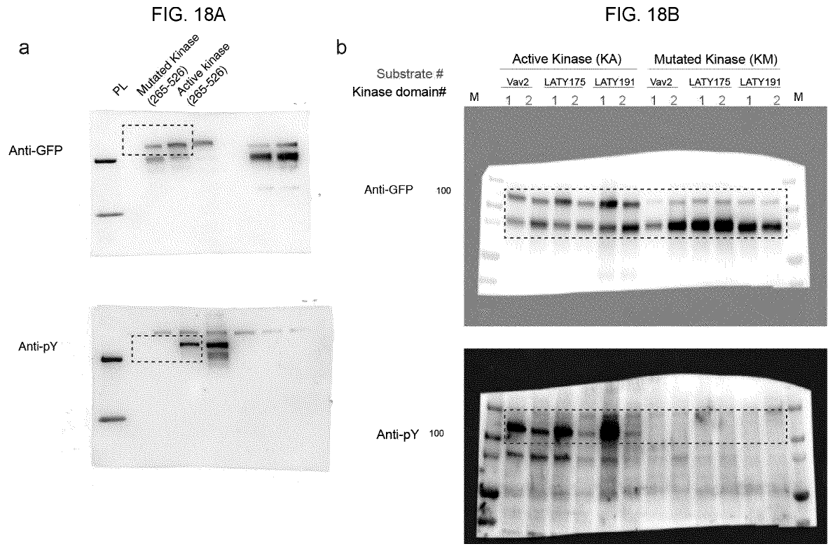

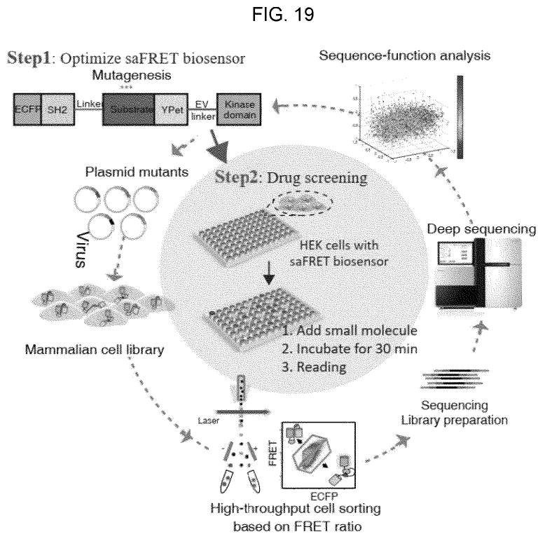

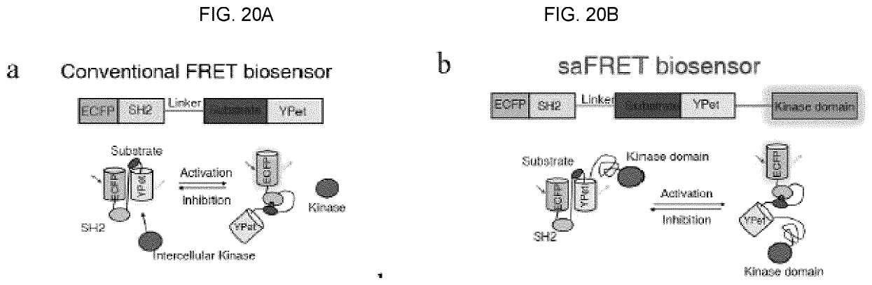

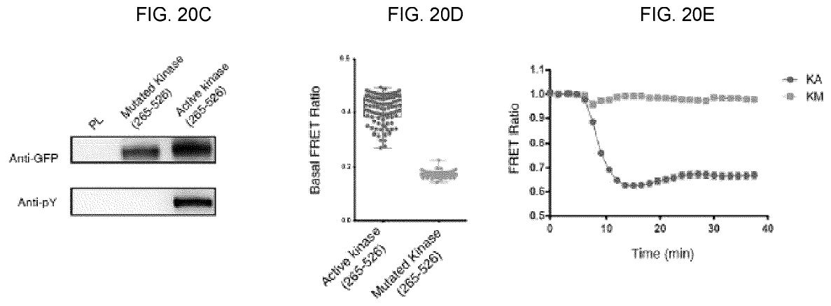

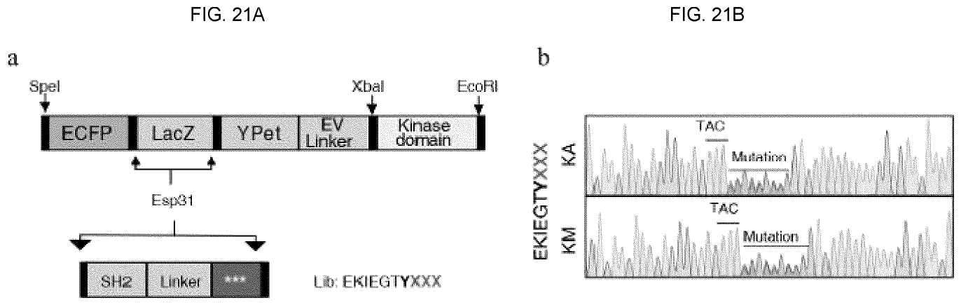

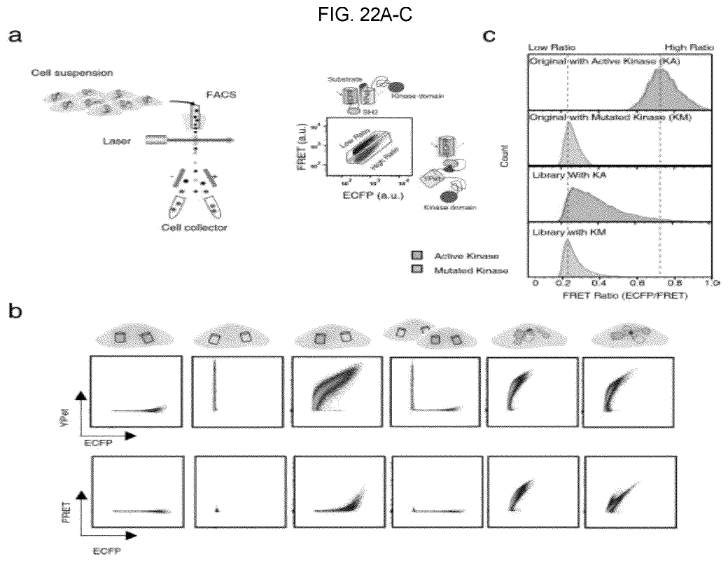

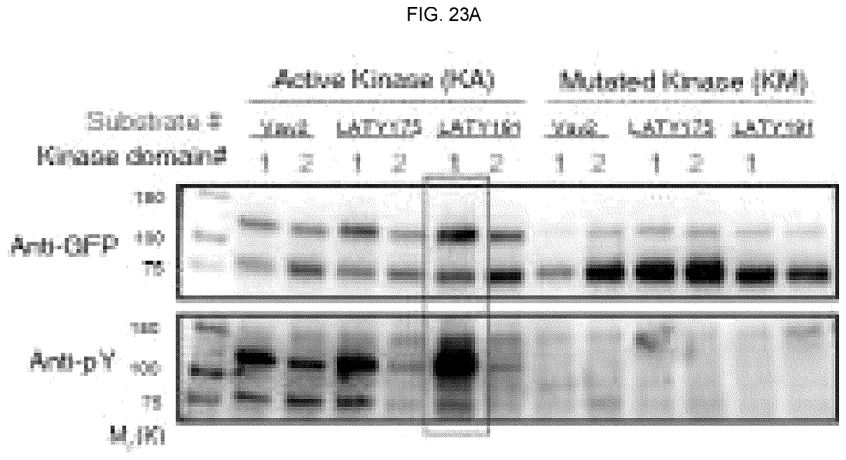

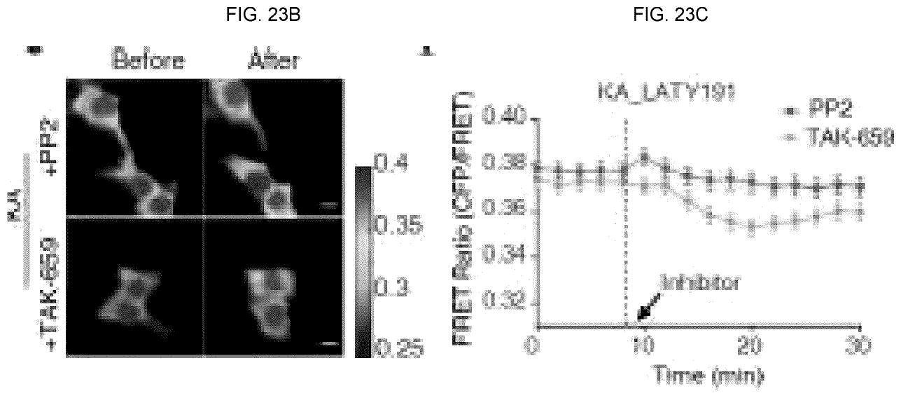

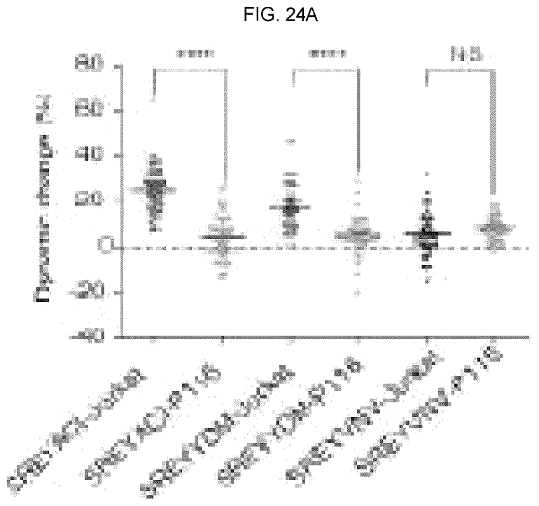

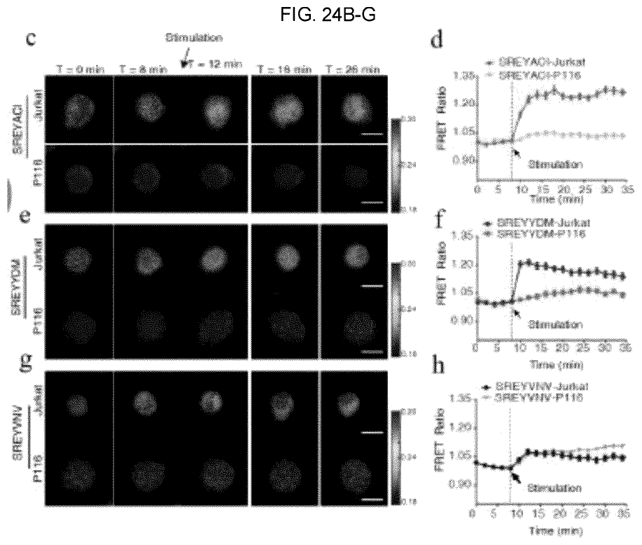

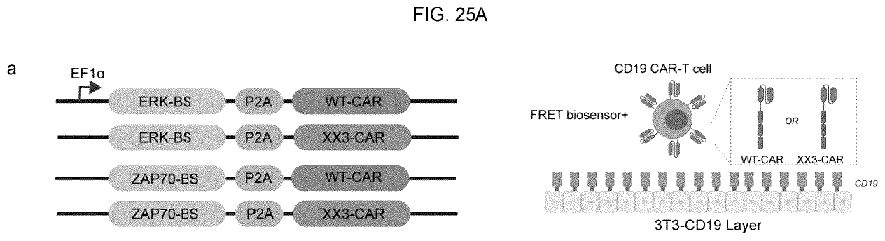

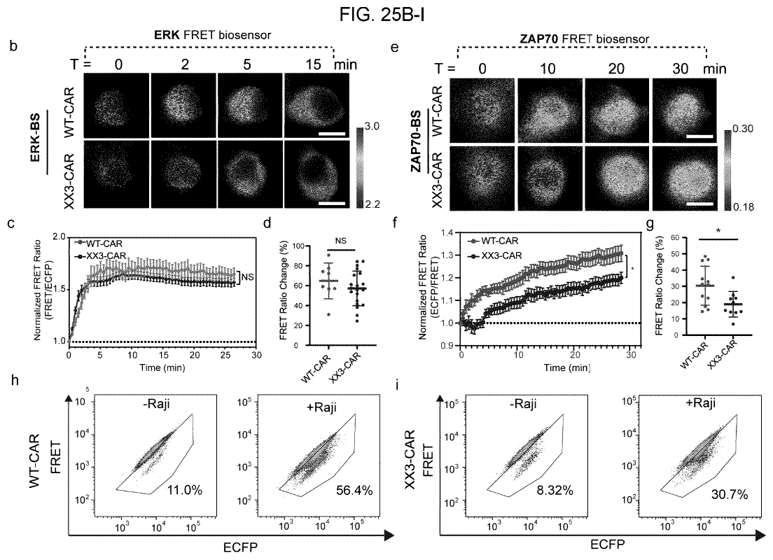

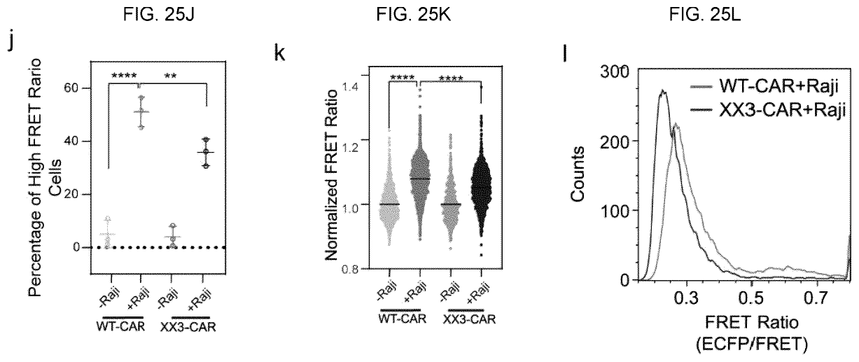

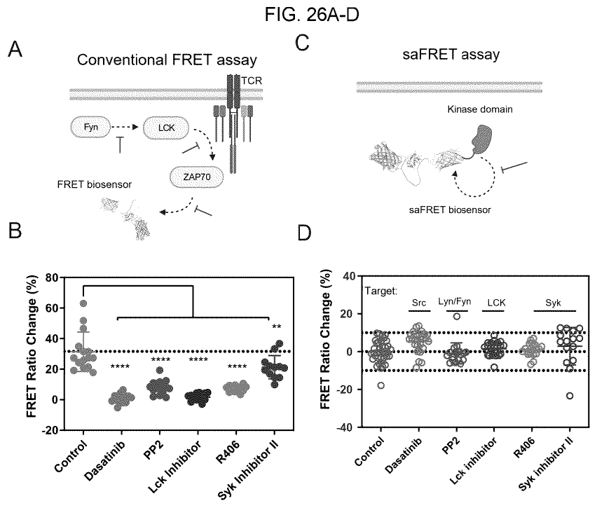

FIG. 8B graphically illustrates quantification of the dynamic ECFP/FRET ratio of the worse biosensor variants tested, the time course of ECFP/FRET ratio of the wild type biosensor before and after PP1 treatment was labeled as a black line,
where biosensor variants tested are EGTYIDI (SEQ ID NO:29), EGTYIDF (SEQ ID NO:37); EGTYWFM (SEQ ID NO:40), EGTYPFT (SEQ ID NO:27), EGTYINF (SEQ ID NO:30), EGTYVQF (SEQ ID NO:36), EGTYHIL (SEQ ID NO: 28), EGTYLLL (SEQ ID NO:26), EGTYVRL (SEQ ID NO:41), EGTYVFW (SEQ ID NO:35), EGTYVFM (SEQ ID NO:34), EGTYRGA (SEQ ID NO:39), EGTYPLM (SEQ ID NO:33), EGTYVGI (SEQ ID NO:31), EGTYDFE (SEQ ID NO: 32), EGTYVEF (SEQ ID NO:38), EGTYGVV (SEQ ID NO:55);

FIG. 8C graphically illustrates the relation between the dynamic range (%) and the product of Eᵥ(KAH) and Eᵥ(KDL), where the dash lines represent the dynamic change (across y-axis) and the value of Eᵥ(KAH)×Eᵥ(KDL) (across x-axis) of wild-type biosensor;

FIG. 8D graphically illustrates the biosensors with different levels of Eᵥ(KAH)×Eᵥ(KDL) were divided into four groups and their time courses accordingly colored with red, pink, light blue, and blue, as discussed in detail in Example 1, below.

FIG. 9A-B illustrates the improvement of the exemplary Fyn FRET biosensor via Lib1:

FIG. 9A graphically illustrates a 4D plot of the enrichment ratio of substrates in different groups for Lib1 (xxxY), in which the amino acid residues before the consensus tyrosine were mutated, the enrichment ratio of the biosensors in the KAH group was color-coded, the substrates satisfying all four criteria were highlighted in color;

FIG. 9B illustrates representative time-lapse images of the parental biosensor and one of the selected biosensors after PP1 treatment, the color bar represents the ECFP/FRET ratio, with hot and cold colors representing the high and low ratios, respectively;

FIG. 9C graphically illustrates quantification of the FRET dynamic change (%) of selected biosensor variants upon PP1 treatment (n>15 in each group), where the selected biosensor variants are YCCYGVV (SEQ ID NO:56); QVYYGVV (SEQ ID NO: 57); DYGYGVV (SEQ ID NO:58); WHYYGVV (SEQ ID NO:59); FHQYGVV (SEQ ID NO:60); IHWYGVV (SEQ ID NO:61); QHMYGVV (SEQ ID NO: 62); GHLYGVV (SEQ ID NO:63); MSVYGVV (SEQ ID NO:64); SDYYGVV (SEQ ID NO:65); HHMYGVV (SEQ ID NO:66); WHMYGVV (SEQ ID NO:67); LIYYGVV (SEQ ID NO:68); MCQYGVV (SEQ ID NO:69); YDQYGVV (SEQ ID NO: 70); NGEYGVV (SEQ ID NO:71); IHFYGVV (SEQ ID NO:72); LSVYGVV (SEQ ID NO:73); YSEYGVV (SEQ ID NO:74); DFHYGVV (SEQ ID NO:75); EGTYGVV (SEQ ID NO:55); INVYGVV (SEQ ID NO:76); ENWYGVV (SEQ ID NO: 77); AVVYGVV (SEQ ID NO:78); QSVYGVV (SEQ ID NO:79); LCGYGVV (SEQ ID NO:80); LAIYGVV (SEQ ID NO:81); IYGYGVV (SEQ ID NO:82); YNSYGVV (SEQ ID NO:83); WYYYGVV (SEQ ID NO:84); PNHYGVV (SEQ ID NO: 85); DQVYGVV (SEQ ID NO:86); QYSYGVV (SEQ ID NO:87); FDIYGVV (SEQ ID NO:88); ESIYGVV (SEQ ID NO:89); IQIYGVV (SEQ ID NO:90); HKFYGVV (SEQ ID NO:91); WQIYGVV (SEQ ID NO:92);

FIG. 9D-E graphically illustrate quantification of the normalized dynamic ECFP/FRET ratio of the better (FIG. 9D) and worse (FIG. 9E) biosensor variants that have been tested, FRET ratio change of the parental biosensor was marked in black line (n≥15 in each group),
where in FIG. 9D the biosensor variants are: NGEYGVV (SEQ ID NO:71), YSEYGVV (SEQ ID NO:74), SDYYGVV (SEQ ID NO:65), LIYYGVV (SEQ ID NO: 68), DYGYGVV (SEQ ID NO:58), WHYYGVV (SEQ ID NO:58), HHMYGVV (SEQ ID NO:66), FHQYGVV (SEQ ID NO:60), GHLYGVV (SEQ ID NO:63), LSVYGVV (SEQ ID NO:73), QHMYGVV (SEQ ID NO:62), QVYYGVV (SEQ ID NO: 57), YCCYGVV (SEQ ID NO:56), WHMYGVV (SEQ ID NO:67), IHWYGVV (SEQ ID NO:61), MCQYGVV (SEQ ID NO:69), YDQYGVV (SEQ ID NO:70), MSVYGVV (SEQ ID NO:64), IHFYGVV (SEQ ID NO:72), DFHYGVV (SEQ ID NO: 75), EGTYGVV (SEQ ID NO:55); and

FIG. 9E graphically illustrates normalized FRET ratios as a function of time for various biosensor variants, the SEQ IDs for the biosensor variants listed above;

FIG. 9F-G illustrate scatter plots of the enrichment ratio of biosensor variants for Ev(KDH) (FIG. 9F) and Ev(KDL) (FIG. 9G), where red and blue dots represent biosensor variants with better and worse performance than the parental biosensor, respectively,
as discussed in detail in Example 1, below.

FIG. 10A-G illustrates the combination of two improved mutants from Lib 1 and Lib2:

FIG. 10A graphically illustrates comparison of the biosensors with combined sequences from both Lib 1 and Lib 2 versus (vs) their parental improved biosensors from either Lib 1 or Lib2, star indicates the biosensors with combined substrate sequences (left columns, sequences NGEYYFF (SEQ ID NO:93), NGEYGVV (SEQ ID NO: 71), EGTYYFF (SEQ ID NO:8)), and the middle columns (DYDYYFF (SEQ ID NO: 94), DYDYGVV (SEQ ID NO:95), EGTYYFF (SEQ ID NO:8)) are improved biosensors from Lib1, and the right columns (YSEYYIF (SEQ ID NO:96), YSEYGVV (SEQ ID NO:74), EGTYYIF (SEQ ID NO:16)) from Lib2; the dashed line indicates the mean FRET change of original WT (EGTYGVV) (SEQ ID NO:55) biosensor; and

FIG. 10B graphically illustrates time courses of the ECFP/FRET ratio signals (normalized FRET ratios) of the combined biosensors (NGEYYFF (SEQ ID NO:93), (DYDYYFF (SEQ ID NO:94), (YSEYYIF (SEQ ID NO:96)) after PP1 treatment,
as discussed in detail in Example 1, below.

FIG. 11A-D illustrate data examining kinase domains and substrates for an exemplary ZAP70 saFRET biosensor:

FIG. 11A illustrates images gels showing the effect of kinase domain on the biosensor phosphorylation: Kinase domain 1: ZAP70 327-619; and Kinase domain 2: ZAP70 327-601;

FIG. 11B graphically illustrates quantification of the dynamic ECFP/FRET ratio changes of exemplary ZAP70 saFRET biosensors with different substrates and kinase domain, upon the treatment by TAK-659 (black-arrow), percentage indicates the reduction (red-arrow) of FRET ratio after TAK-659 treatment; and

FIG. 11C-D illustrate representative images (FIG. 11C) and graphically illustrates time courses (FIG. 11D) of the ECFP/FRET ratio signals of an exemplary ZAP70 saFRET biosensor with different inhibitors, TAK-659; and PP2, a Src family kinase inhibitor,
as discussed in detail in Example 1, below.

FIG. 12A-B illustrate data showing unbiased library generation for an exemplary ZAP70 biosensor:

FIG. 12A graphically illustrates sequencing results of library 1 (Lib1) with active (KA) or dead kinase (KD), TAC encodes for tyrosine; and

FIG. 12B graphically illustrates sequencing results of library 2 (Lib2) with active (KA) or dead kinase (KD),
as discussed in detail in Example 1, below.

FIG. 13A-D illustrate data showing the mutation of amino acid residues upstream to the consensus tyrosine in the substrate of the biosensors:

FIG. 13A illustrates a 4D plot of the enrichment ratio of substrates from different groups; the enrichment ratio in the KAH group is color-coded, and the substrates satisfying all four criteria were highlighted with color;

FIG. 13B graphically illustrates a scatter plot of biosensors with different substrates, where the biosensor variants with the top 10 products of Eᵥ(KAH) and Eᵥ(KDL) from Lib1 were labeled in Red (better biosensors than the parental biosensor) or Blue (worse biosensors than the parental biosensor);

FIG. 13C graphically illustrates quantification of the dynamic change of biosensor variants (HGDYVNV (SEQ ID NO:97), CYPYVNV (SEQ ID NO:98), GDDYVNV (SEQ ID NO:99), MGDYVNV (SEQ ID NO:100), DFEYVNV (SEQ ID NO: 101), YGDYVNV (SEQ ID NO:102), CSDYVNV (SEQ ID NO:103), HDDYVNV (SEQ ID NO:104), DGDYVNV (SEQ ID NO:105)), upon PP1 treatment; and

FIG. 13D graphically illustrates quantification of the normalized dynamic ECFP/FRET ratio of the selected biosensor variants (SEQ ID NO:s listed in FIG. 13C), and FRET ratio change of the parental biosensor was marked in black line,
as discussed in detail in Example 1, below.

FIG. 14A-F illustrate verification of the improved biosensors in primary human CD4+ T cells:

FIG. 14A graphically illustrates dynamic ranges of exemplary ZAP70 biosensors (SREYACI (SEQ ID NO:44), SREYYDM (SEQ ID NO:45)) with different substrates, SREYVNV (SEQ ID NO:53) represents the parental biosensor;

FIG. 14B-C illustrate time courses (FIG. 14B) and time-lapse images (FIG. 14C) and of the SREYYDM (SEQ ID NO:45) biosensor before and after TCR activation induced by CD3/CD28 antibody stimulation;

FIG. 14D schematically illustrates a design of an exemplary membrane-bound ZAP70 FRET biosensors and their membrane localization in HEK cells;

FIG. 14E illustrates representative time-lapse images of ZAP70 activity change in different membrane compartments after TCR activation in primary human T cells; color bar indicates ECFP/FRET intensity ratio, with hot and cold colors representing the high and low ratios, respectively; and

FIG. 14F illustrates time courses of normalized ECFP/FRET ratio of an exemplary ZAP70 FRET biosensor in different membrane compartments, as discussed in detail in Example 1, below.

FIG. 15A-D illustrate stable HEK 293T cell line with exemplary ZAP70 saFRET biosensor for HTDS assay targeting an exemplary ZAP70 kinase;

FIG. 15A schematically illustrates the advantage of imaging adherent cells compared to suspension cells in general imaging platforms, suspension cells, such as immune cells, float freely in media, and the focus or the observation field can easily become lost over time, especially at high magnification scale during imaging;

FIG. 15B illustrates cell sorting of the stable HEK293T cell line with a similar expression level of an exemplary ZAP70 saFRET biosensor, these sorted cells are used for HTDS assay;

FIG. 15C graphically illustrates the isolated stable HEK cell line expressing an exemplary ZAP70 saFRET biosensor demonstrated approximately 25% change after a high-dose 25 μM TAK659 treatment; and

FIG. 15D illustrates representative ECFP/FRET ratio images of an exemplary ZAP70 saFRET biosensor after 25 μM TAK659 treatment,
as discussed in detail in Example 1, below.

FIG. 16A-B illustrate HTDS using exemplary ZAP70 saFRET biosensors:

FIG. 16A graphically illustrates a concentration-dependent response of saFRET biosensor to TAK-659, the 10 μM TAK659 treatment could not reduce the FRET ratio significantly; and

FIG. 16B top panel schematically illustrates a design of an exemplary ZAP70 biosensor with kinase-dead domain (saFRETkd); and

FIG. 16B bottom panel graphically illustrates the FRET ratio changes of a saFRET biosensor with kinase-dead domain in counter screening, small molecules which have non-specific effects on FRET signals are eliminated in this step, as discussed in detail in Example 1, below.

FIG. 17A-B illustrate data showing taht staurosporine and AZD7762 are potent inhibitors of ZAP70 signaling pathway:

FIG. 17A illustrates representative images of pZAP70 (Y493) in Jurkat T cells with different treatments; and

FIG. 17B illustrates representative images of pZAP70 (Y493) in P116-ZAP70-R360P cells with different treatment,
as discussed in detail in Example 1, below.

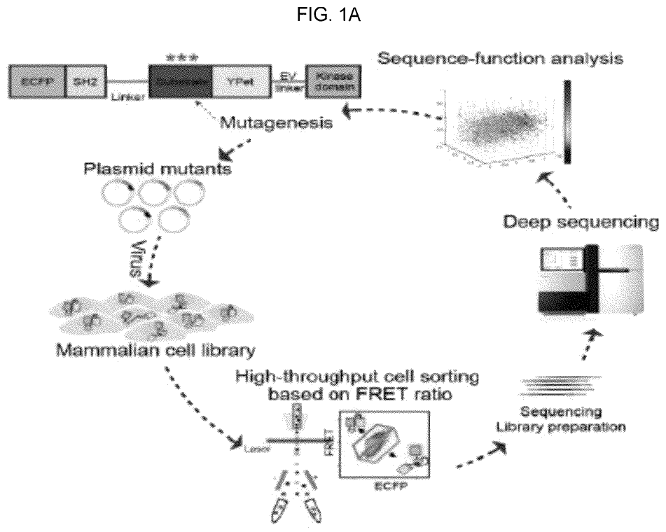

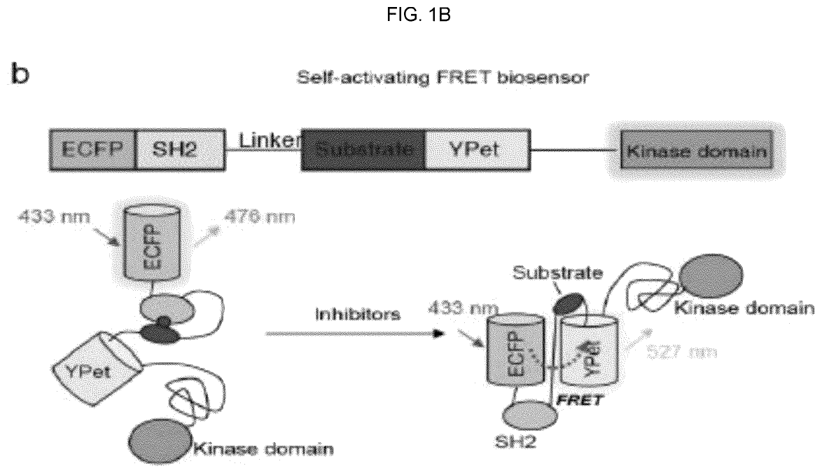

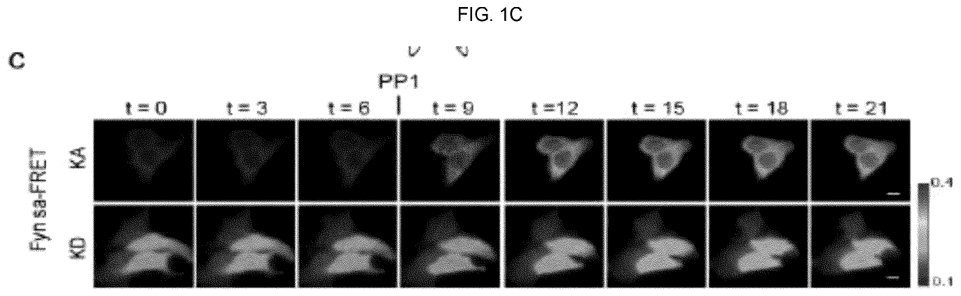

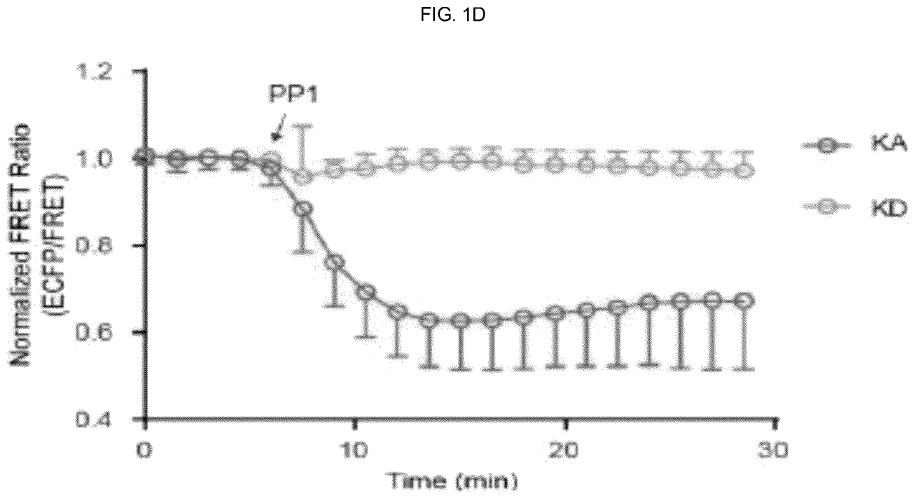

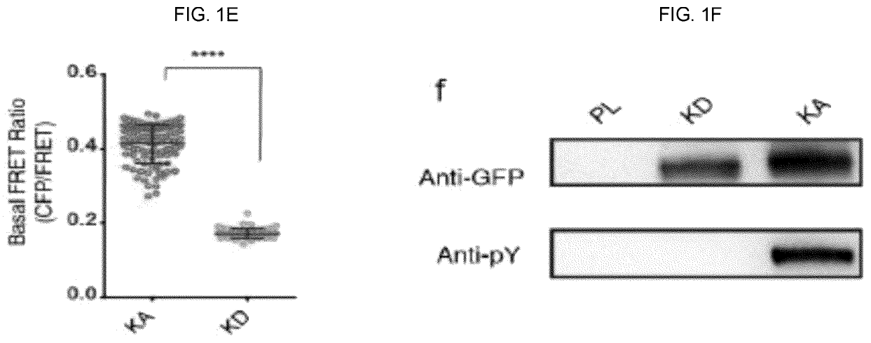

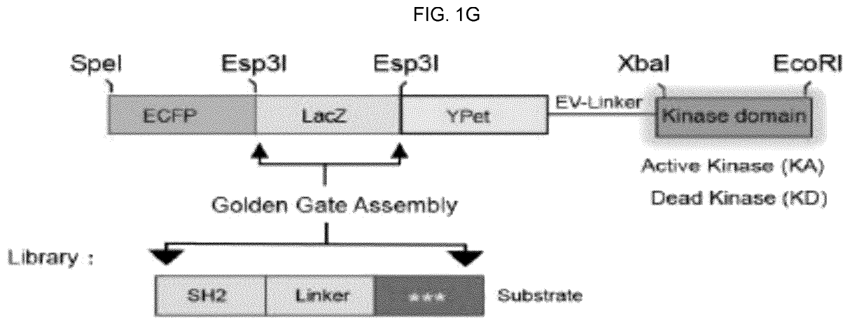

FIG. 18A-B illustrates images of Western blots, the dash line indicates the cropped regions in FIG. 1A (FIG. 18A) and FIG. 9B (FIG. 18B), respectively, as discussed in detail in Example 1, below.

FIG. 19 schematically illustrates saFRET biosensor optimization by use of directed evolution for drug screening, as discussed in detail in Example 2, below.

FIG. 20A-E illustrate design and validation of exemplary saFRET biosensors:

FIG. 20A schematically illustrates a conventional saFRET biosensor;

FIG. 20B schematically illustrates an exemplary saFRET biosensor as provided herein, an engineered self-activating FRET (saFRET) biosensor with an additional kinase domain; and

FIG. 20C-E illustrate data showing that the FRET change was specifically mediated by the kinase domain in HEK293 cells, and if the active kinas domain is replaced with a kinase-dead domain, the phosphorylation in the peptide (imaged in FIG. 20C) and the FRET ratio significantly reduced both in endpoint cell imaging assay (graphically illustrated in FIG. 20D) and live cell imaging (graphically illustrated in FIG. 20E),
as discussed in detail in Example 2, below.

FIG. 21A-B illustrate modularized templates for library generation:

FIG. 21A schematically illustrates generation of a DNA library using a fully modularized template; for the Fyn biosensor, three positions of the amino acid after the tyrosine site in the substrate were randomly mutated, the bottom panel illustrates a PCR product using an NNK primer, the illustrated peptide is EKIEGTYXXX (SEQ ID NO: 2);

FIG. 21B graphically illustrates verification of random mutagenensis using sanger sequence,
as discussed in detail in Example 2, below.

FIG. 22A-C illustrate mammalian cell library screening by FACS:

FIG. 22A schematically illustrates an exemplary mammalian cell library screening by FACS, where FRET ratio of the FRET biosensors can be measured in individual cells;

FIG. 22B illustrates different control groups in a FACS experiment: from left to right: ECFP only, YPet only, ECFP and YPet mixture in cells, ECFP cell and YPet cell mixture, cells with KA FRET biosensor, cell with KM FRET biosensor;

FIG. 22C graphically illustrates cell selection strategy in FACS experiments, where only the top 5% of the cells were selected and subjected to analysis,
as discussed in detail in Example 2, below.

FIG. 23A illustrates an image of Western blots of kinase domains and substrates: kinase domain 1:327 to 619, and kinase domain 2:327-601, as explained in detail in Example 2, below.

FIG. 23B-C illustrate representative images (FIG. 23B) and quantification results (FIG. 23C) of live cell imageing of sa-FRET biosensor of an exemplary Zap70 with different inhibitors; Zap70 saFRET biosensor could respond to TAK-659, a Zap70 kinase inhibitor rather than PP2, an Sre family kinase inhibitor, as explained in detail in Example 2, below.

FIG. 24A illustrates the dynamic range of different biosensors in human T cells: SREYACI (SEQ ID NO:44), SREYYDM (SEQ ID NO:45), SREYVNV (SEQ ID NO: 53), as explained in detail in Example 2, below.

FIG. 24B-G illustrate representative time lapse images (FIG. 24B, FIG. 24D, FIG. 24F) and graphic representation of FRET ratio dynamic change (FIG. 24C, FIG. 24E, FIG. 24G) (SREYACI (SEQ ID NO:44), SREYYDM (SEQ ID NO:45), SREYVNV (SEQ ID NO:53)) of different biosensors before and after TCR activation induced by CD3/CD28 antibody clusters.

FIG. 25A-L illustrate data showing less ZAP70 activation in XX3-CD19 CAR-T cells:

FIG. 25A is a schematic drawings of constructs: the ERK- or ZAP70-FRET biosensor was co-expressed with WT-CAR or XX3-CAR in Jurkat T cells; in XX3-CAR, the tyrosine in the first and second ITAM motif was mutated to phenylalanine; the CAR T cells were then dropped onto the 3T3 cells that constitutively express CD19 to monitor the dynamic ZAP70 or ERK kinase activations;

FIG. 25B illustrates representative images of ERK biosensor in T cells after attaching to the 3T3 CD19+ cell monolayer, scale bar=10 μm;

FIG. 25C graphically illustrates time courses of FRET ratio (FRET/CFP) of ERK biosensor in WT- or XX3-CAR-T cells (N=8 and 20 in each group), error bars, mean±SEM;

FIG. 25D graphically illustrates percentage changes of ERK-FRET biosensor in WT- or XX3-CAR-T cells (Unpaired two-tailed Student's t-test, NS, P>0.05), error bars, mean±SD;

FIG. 25E illustrates representative images of ZAP70 biosensor in T cells after attaching to the 3T3 CD19+ cell monolayer, scale bar=10 μm;

FIG. 25F graphically illustrates time courses of FRET ratio (ECFP/FRET) of ZAP70 biosensor in WT CAR-T or XX3-CAR-T cells (N=12 and 10 in each group), error bars, mean±SEM;

FIG. 25G graphically illustrates percentage changes of ZAP70-FRET biosensor in WT CAR-T or XX3-CAR-T cells (Unpaired two-tailed Student's t-test, *P=0.017), error bars, mean±SD;

FIG. 25H graphically illustrates flow-cytometry analysis of ECFP/FRET in WT-CAR T cells before and after CD19+ Raji cell stimulation;

FIG. 25I graphically illustrates flow-cytometry analysis of ECFP/FRET in XX3-CAR T cells before and after CD19+ Raji cell stimulation;

FIG. 25J graphically illustrates percentage of High-FRET ratio cells in different groups of three independent experiments, (One-way ANOVA, ****p<0.0001, **P=0.0054), error bars, mean±SD;

FIG. 25K graphically illustrates normalized FRET ratio of WT- or XX3 CAR-T cells before and after CD19+ Raji cell stimulation. (One-way ANOVA, ****P<0.0001), error bars, mean±SEM; and

FIG. 25L graphically illustrates a histogram of FRET ratio in WT- or XX3 CAR-T cells after CD19+ Raji cell stimulation,
as described in detail in Example 3, below.

FIG. 26A-D illustrate that the FRET change induced by the identified inhibitors was specifically mediated by the change of the ZAP70 kinase domain:

FIG. 26A schematically illustrates a conventional FRET assay;

FIG. 26B graphically illustrate FRET ratio changes using the conventional FRET assay;

FIG. 26C schematically illustrates modified, engineered saFRET assay as provided herein; and

FIG. 26D graphically illustrate FRET ratio changes using a saFRET assay as provided herein;

as discussed in Example 3, below.

Like reference symbols in the various drawings indicate like elements.

### DETAILED DESCRIPTION

In alternative embodiments, provided are self-activating Förster resonance energy transfer (saFRET) biosensors, and methods for making and using them. In alternative embodiments, provided are methods encompassing a systematic approach that couples FRET and sequencing (FRET-Seq) to integrate random mutagenesis, fluorescence-activated cell sorting (FACS), and next-generation sequencing (NGS) to screen and identify sensitive biosensors from large-scale libraries directly in mammalian cells, utilizing the design of self-activating FRET (saFRET) biosensors as provided herein.

In alternative embodiments, provided herein is a new platform for screening drugs that incorporate use of protein kinases (for example, Fyn and ZAP70 kinases) in the self-activating FRET (saFRET) biosensor as provided herein. In this invention, we rationally designed a saFRET biosensor by linking an active kinase domain to the conventional FRET biosensor to achieve intermolecular activation and made it suitable for high throughput drug screening (HTDS) after optimization by using a directed-evolution platform.

In alternative embodiments, a kinase domain is directly fused to a FRET biosensor, which allows the screening of drugs targeting the kinase in a cell (for example, a HEK cell), minimizing the effect of the heterogeneity of individual cells due to the endogenously expressed kinases. The advantage of our saFRET is also that it can be used to screen drugs for any kinases that express either in adherent or suspension cells. For some kinases such as Zap70, the expression is relatively restricted to suspension cells (for example, T cells) and make it difficult to screening drugs using the conventional FRET biosensor. The high performance of saFRET biosensor enables us to screen small molecules that target any kinase expressed only in suspension cells in adherent cells (for example, HEK293 cells) by using the imaging platform in a short period (within one hour). We have demonstrated that this design has high specificity and sensitivity since it has less chance to be influenced by other signaling pathways in HEK cells.

The saFRET-based biosensors as provided herein also can present a number of significant advantages over technologies that are based on the antibody-detection or biochemical binding assays. In the FRET technology, two fluorescent images from the donor and acceptor emissions are obtained simultaneously to calculate the ratio to represent the molecular activity. This ratiometric FRET imaging reduces the noise engendered from variations of the protein/peptide expression and concentration, the cell size and thickness, and the intensity of the excitation light source, as well as the instability of optical devices. Hence, the FRET signals can provide a much higher level of accuracy, comparing to the antibody-based or other protein-protein/peptide binding approaches.

To create a high performance saFRET for a specific kinase, we have developed the new method for FRET biosensor optimization based on directed evolution. The directed-evolution platform provides a systematic and general approach for optimizing the biosensor in mammalian cells. The most innovative aspect of this platform is the systematic approach for the direct screening of optimized FRET biosensors that are capable of detecting, in principle, any post-translational modification, with the domains orthogonal to the endogenous signaling molecules. At the current stage, the optimization of FRET biosensors in their sensitivity and specificity is rather semi-rational and labor-intensive, mostly in a trial-and-error fashion, for example, see Ibraheem, Yap et al. 2011, Komatsu, Aoki et al. 2011, Piljic, de Diego et al. 2011, Lam, St-Pierre et al. 2012. In contrast, in platforms as provided herein, we integrate site-saturated mutagenesis, mammalian cell library, fluorescence-activated cell sorting (FACS), and NGS together in a framework of directed evolution to optimize the FRET biosensors. Although directed evolution and FACS have been employed to develop FPs with novel properties and binding pairs with high affinities (see for example, Shaner, Campbell et al. 2004, Nguyen and Daugherty 2005, Shaner, Lin et al. 2008), there has been no general method established to optimize FRET biosensors, as proposed here directly in mammalian cells; thus, the direct screening of FRET biosensor libraries in mammalian cells as provided herein is novel. We fuse the kinase domain with a linker at the C-terminus of the biosensors to allow the substrate phosphorylation by the intramolecular kinase domain and, subsequently, the conformational changes of sensing unit, which can lead to the FRET signal readouts for screening. The individual mammalian cells hosting biosensor libraries can hence be sorted by FACS based on FRET signals to identify favorable substrates of the target kinase as well as their efficient binding domains at the same time. The sequences of the substrate and binding domain can be revealed by amplicon production and NGS sequencing systematically.

Exemplary biosensors of Fyn and ZAP70 kinases exhibit high performance and have enabled the dynamic imaging of T-cell activation mediated by T-cell receptors (TCRs) and chimeric antigen receptors (CARs). A high-throughput drug screening (HTDS) assay of a kinase inhibitor library based on the improved saFRET biosensors further allowed the identification of compounds that demonstrated novel efficiency in inhibiting ZAP70 kinase activity and disease-related T-cell activation. Thus, the compositions and products of manufacture as provided herein comprising saFRET biosensors as provided herein have been demonstrated as an effective platform to screen large-scale biosensor libraries in mammalian cells for cellular imaging and drug screening.

As described in Example 1, below, utilizing a self-activating FRET (saFRET) biosensor fused to an active kinase domain, we have developed a new method to couple FRET signals to next generation sequencing (NGS) (FRET-Seq) of biosensor libraries in mammalian cells for the improvement of biosensor performance (FIG. 1A). This FRET-Seq platform was applied to systematically improve both Fyn and ZAP70 biosensors in a high throughput fashion. The improved ZAP70 biosensor and its corresponding saFRET were applied to single T cell and CAR-T cell imaging and a HTDS assay of compound libraries for the identification of efficient ZAP70 kinase inhibitors that can suppress T cell activations engendered from pathological ZAP70 mutations. The saFRET biosensor design also enables stable phosphorylation/activation of the biosensor dominated by the corresponding active kinase domain, allowing HTDS of ZAP70 inhibitors in HEK cells to avoid the interference of ZAP70 upstream kinases as in T cells of conventional drug screen assays.

### Products of Manufacture and Kits

Provided are compositions and products of manufacture and kits comprising saFRET biosensors as provided herein, which are used for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Making and Using Exemplary Self-Activating FRET (saFRET) Biosensors

This example demonstrates that methods and compositions as provided herein using the exemplary Self-Activating FRET (saFRET) biosensors as provided herein are effective and can provide an effective, systematic approach coupling FRET and sequencing (FRET-Seq) to integrate random mutagenesis, fluorescence-activated cell sorting (FACS), and next-generation sequencing (NGS) to screen and identify sensitive biosensors from large-scale libraries directly in mammalian cells.

Utilizing exemplary self-activating FRET (saFRET) biosensors fused to an active kinase domain, we have developed a new method to couple FRET signals to next generation sequencing (NGS) (FRET-Seq) of biosensor libraries in mammalian cells for the improvement of biosensor performance (FIG. 1A). This FRET-Seq platform was applied to systematically improve both Fyn and ZAP70 biosensors in a high throughput fashion. The improved ZAP70 biosensor and its corresponding saFRET were applied to single T cell and CAR-T cell imaging and a HTDS assay of compound libraries for the identification of efficient ZAP70 kinase inhibitors that can suppress T cell activations engendered from pathological ZAP70 mutations. The saFRET biosensor design also enables stable phosphorylation/activation of the biosensor dominated by the corresponding active kinase domain, allowing HTDS of ZAP70 inhibitors in HEK cells to avoid the interference of ZAP70 upstream kinases as in T cells of conventional drug screen assays.

### Results

Engineering of Fyn saFRET Biosensor

A kinase FRET biosensor was constructed to contain a tyrosine substrate peptide and a Src Homology 2 (SH2) domain as the sensing unit, and a FRET pair of fluorescent proteins (FPs) as the reporting unit. The FRET efficiency of the two FPs can be modulated by the sensing unit². To minimize the impact of noise introduced by heterogeneously expressed kinases in host cells, a self-activating FRET (saFRET) biosensor was constructed by fusing an active kinase domain to the biosensor via an EV linker to allow self-activation to dominate the FRET signals (FIG. 1B). For Fyn saFRET biosensor, a Fyn kinase domain was linked to a Fyn-kinase-specific peptide substrate (EKIEGTYGVV (SEQ ID NO:112), from p34cdc2)²⁷. Replacement of the kinase domain by its kinase-dead version abolished the FRET ratio and the Fyn inhibitor PP1-induced dynamic changes of the saFRET biosensor during live-cell imaging (FIG. 1C-E), as well as the biosensor phosphorylation (FIG. 1f), indicating that the FRET change of the saFRET biosensor is specifically mediated by the active kinase domain. With the modular design of its sensing unit, this functional Fyn-saFRET biosensor was utilized to create a template for the library generation (FIG. 1G).

### FRET Screening and FACS Sorting of Biosensors in Mammalian Cells

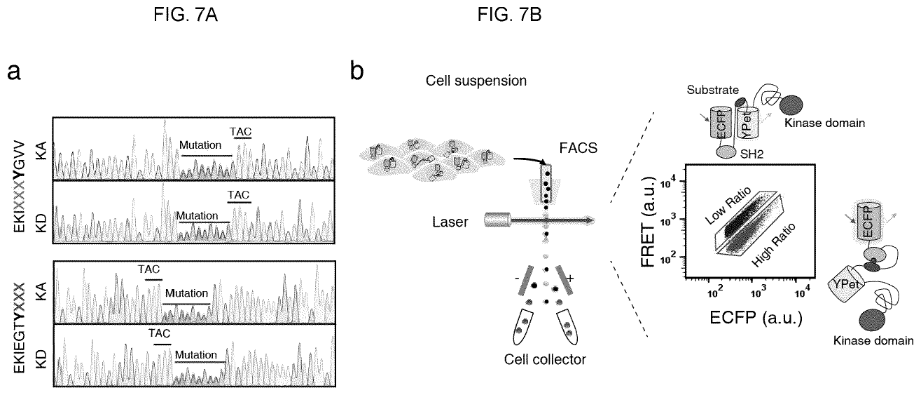

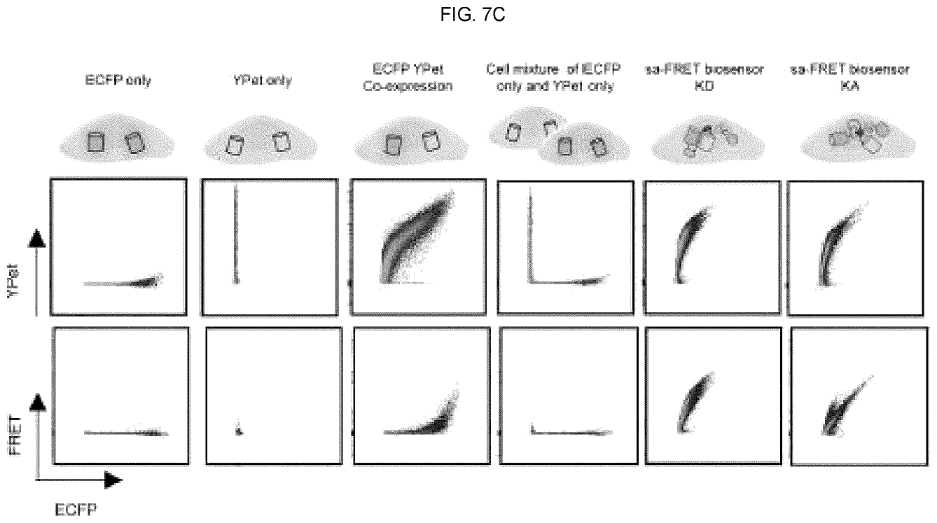

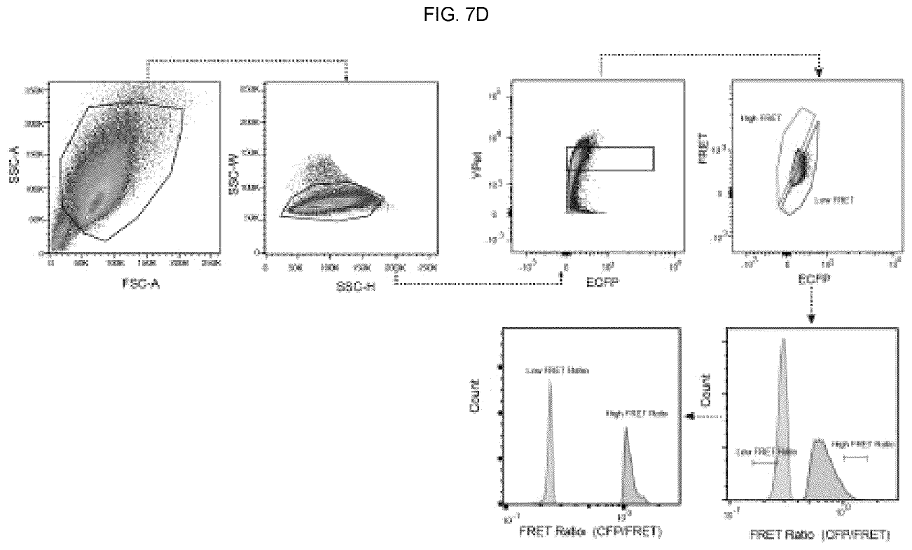

Since the amino acids surrounding the consensus tyrosine residue of the substrate are important in being recognized by the corresponding kinase²⁸ and the SH2 domain^{29, 30}, these neighboring residues were subjected to site-saturation mutagenesis separately to create two biosensor libraries (Lib1: −1, −2, −3, Y or Lib2: Y, +1, +2, +3) by using degenerate primers (NNK) 31, with each library consisting of 32,768 variants (Supplementary FIG. 1a, or FIG. 7A). To reduce false positive selection, we also generated control libraries with dead kinase (K299M mutation³²) for counter screening. After the generation of a mammalian cell library by infection with viral libraries, individual cells expressing biosensor variants were sorted into low and high FRET ratios (ECFP/FRET ratios) by FACS (Supplementary FIG. 1b-d, or FIG. 7B-D). Then, the sequences of selected biosensor variants were decoded by NGS (see supplementary methods: NGS sequencing).

### Identifying the Sequences of Desired Biosensor Variants

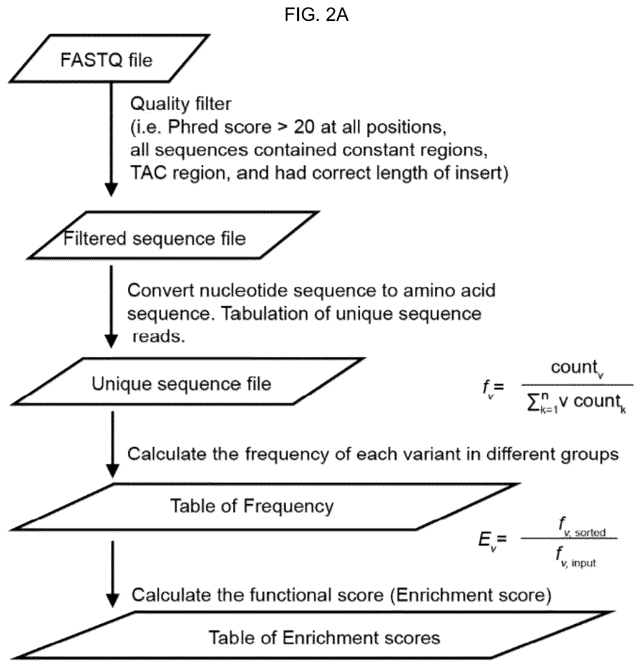

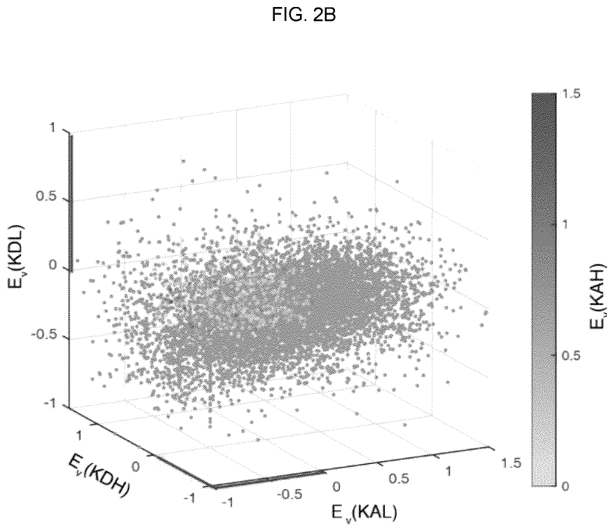

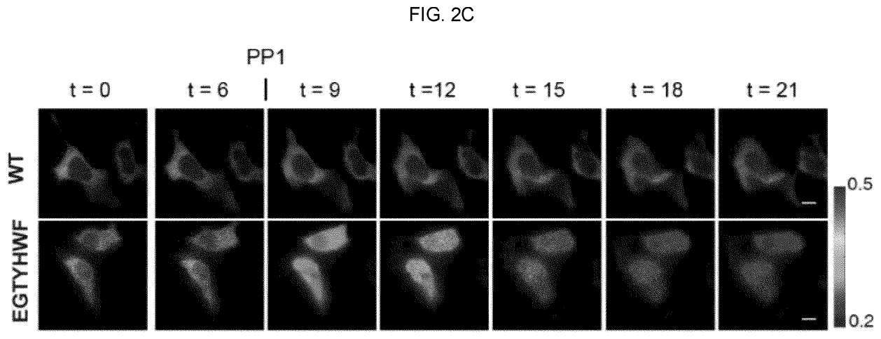

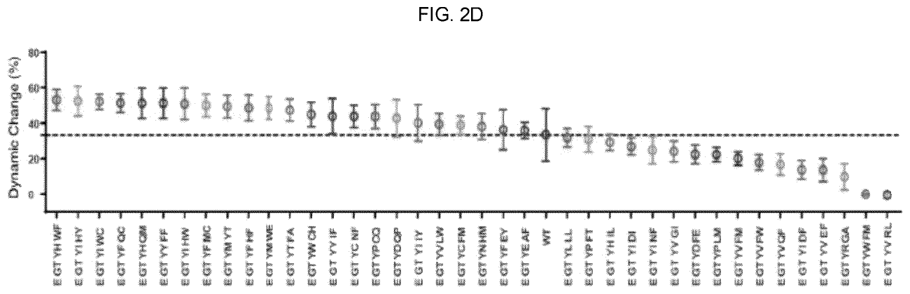

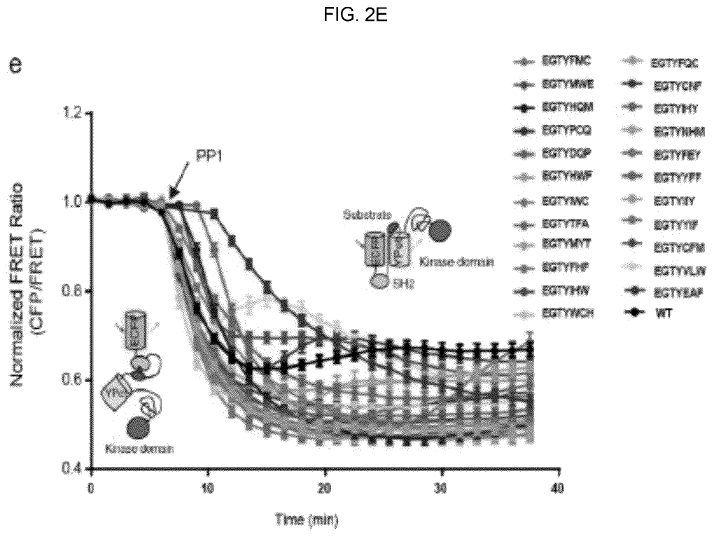

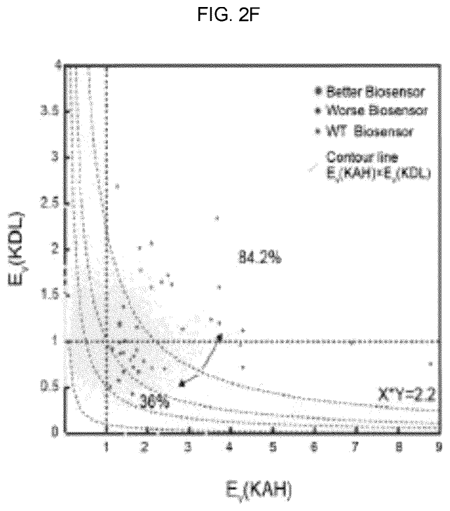

FACS screening and sorting enriches cells containing the desired biosensor variants. The change in frequency of each variant sequence between the FACS-sorted groups and their input control before sorting can represent the enrichment of the variant by sorting^{33,34}, which can be quantified by calculating the enrichment ratio (Eᵥ) of each variant after sorting (See supplementary method for details: Sequencing analysis). Since the ECFP/FRET ratio of the saFRET biosensor depends on its phosphorylation by kinase domain, the ECFP/FRET ratio of the desired saFRET biosensor variants should be high with an active kinase domain, but low with a kinase-dead domain³⁵. As such, the most sensitive biosensors can be enriched in (1) a high-ratio sorted saFRET library with Active Kinase domain (KAH), and (2) a low-ratio sorted library with Kinase-Dead domain (KDL). These desired biosensors should not be enriched in two other libraries, viz. (3) the low-ratio sorted library with an active kinase (KAL) and (4) the high-ratio sorted library with a kinase-dead domain (KDH). Using this multiplex sorting strategy in combination with NGS and analysis, we acquired an average of 16 million reads per library and converted the raw reads into amino acid sequences (FIG. 2a). We then applied the four selection criteria to select biosensor sequences, as illustrated in the four-dimensional plot (FIG. 2b). We expect to systematically assess all the library variants to identify sensitive biosensors with the substrate sequences that allow (a) favorable phosphorylation by the corresponding kinase and (b) efficient binding to the intramolecular SH2 domain upon phosphorylation.

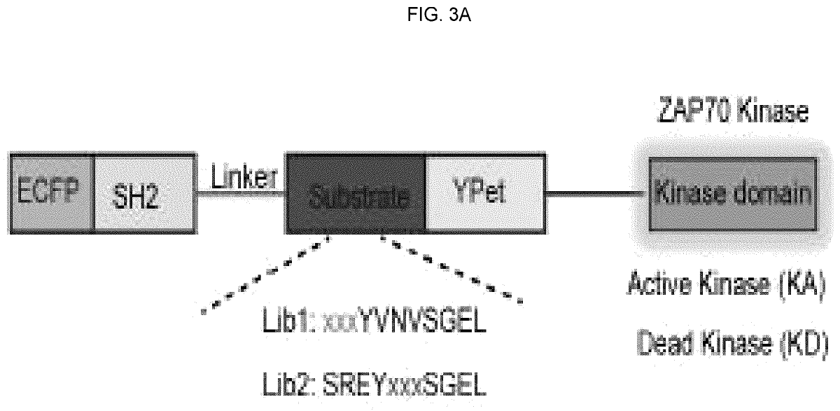

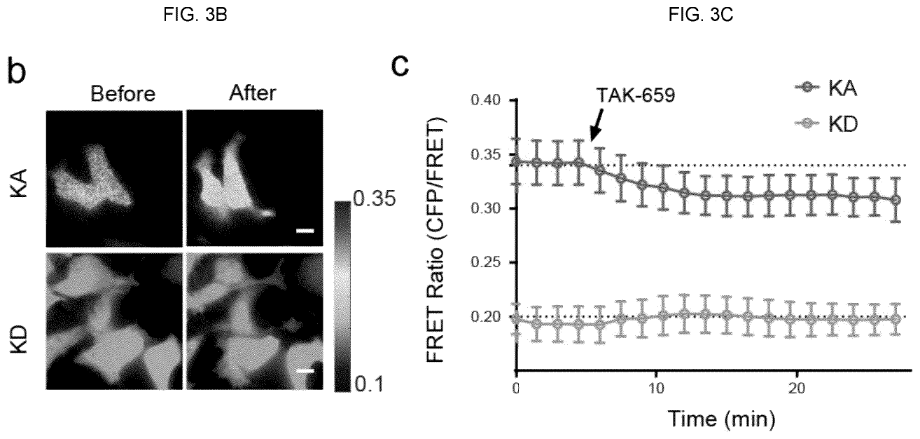

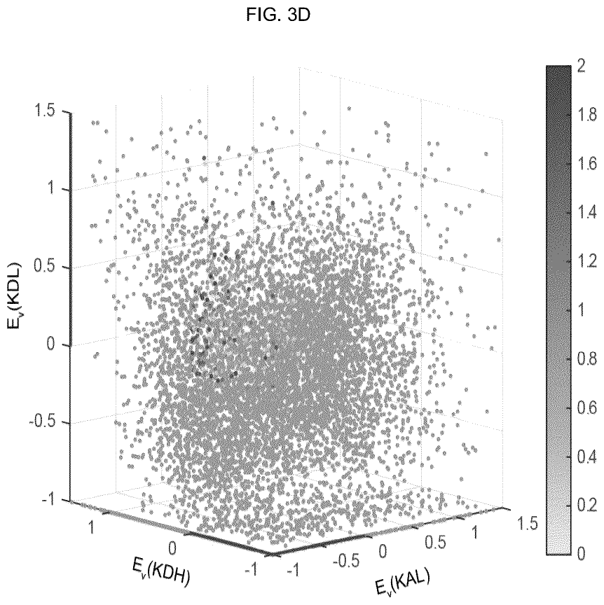

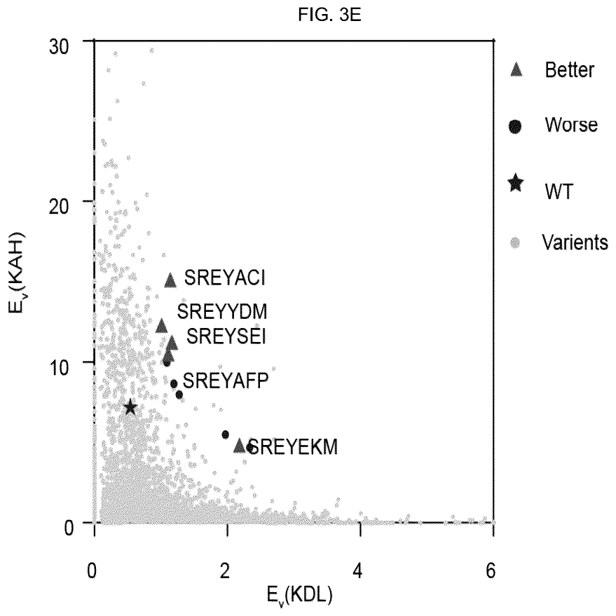

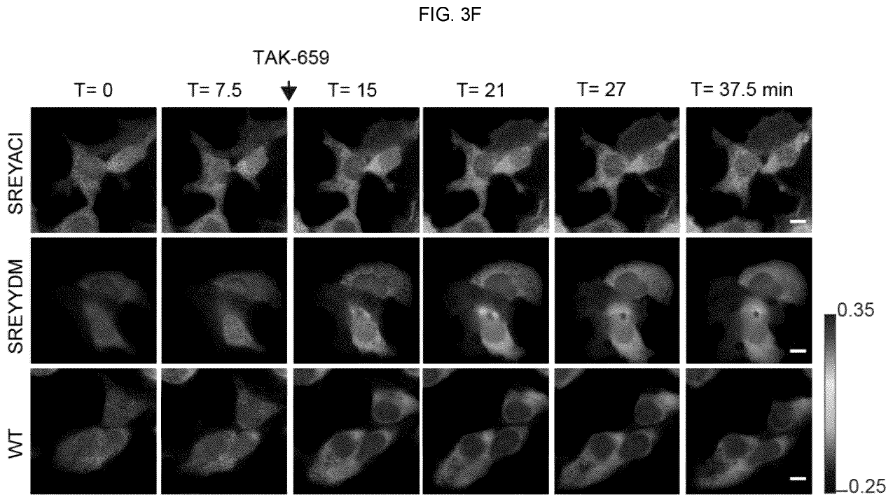

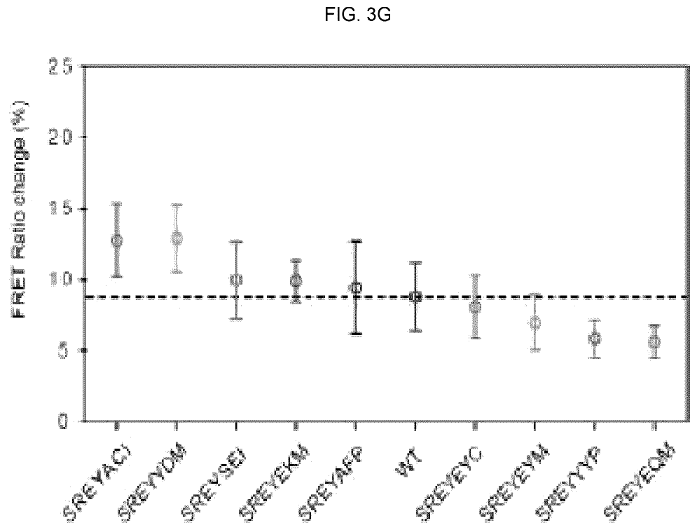

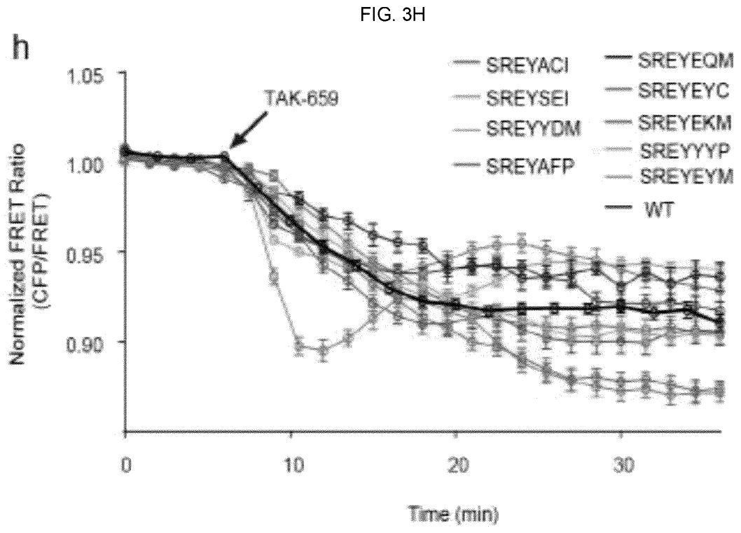

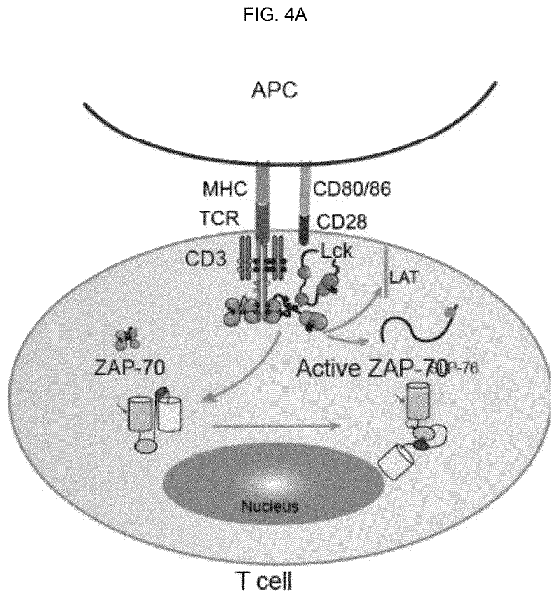

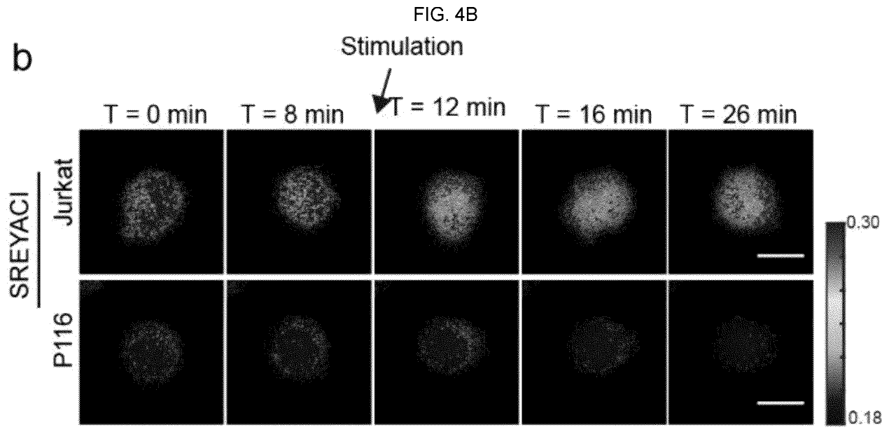

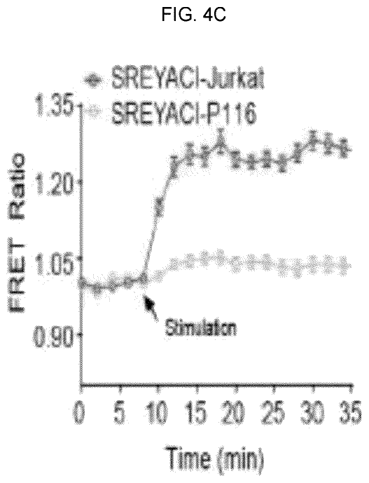

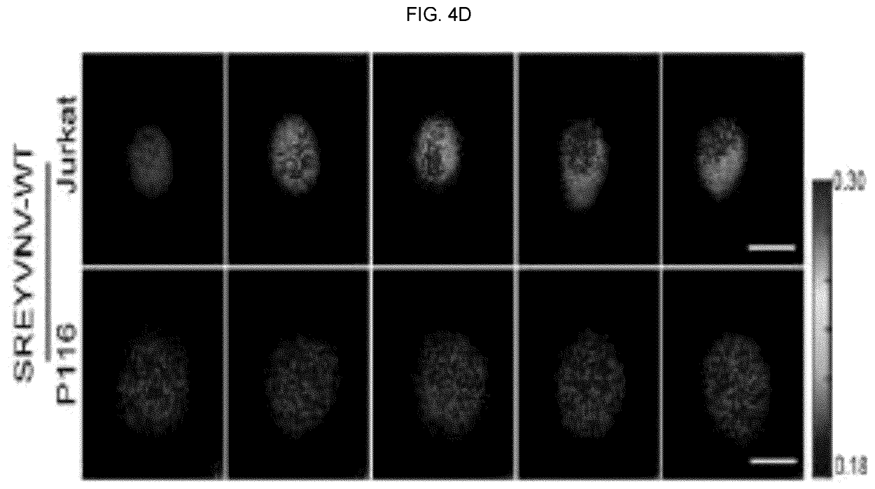

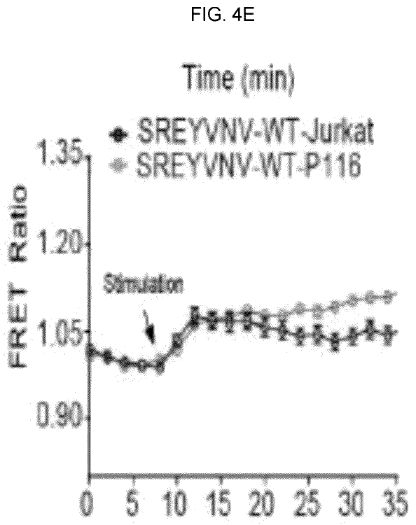

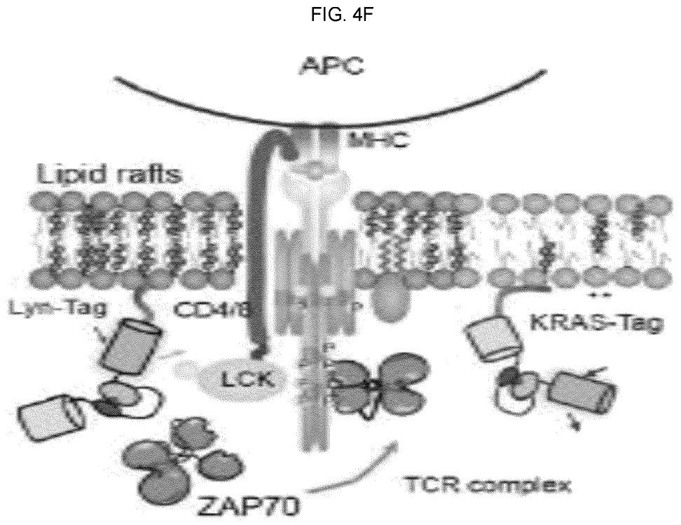

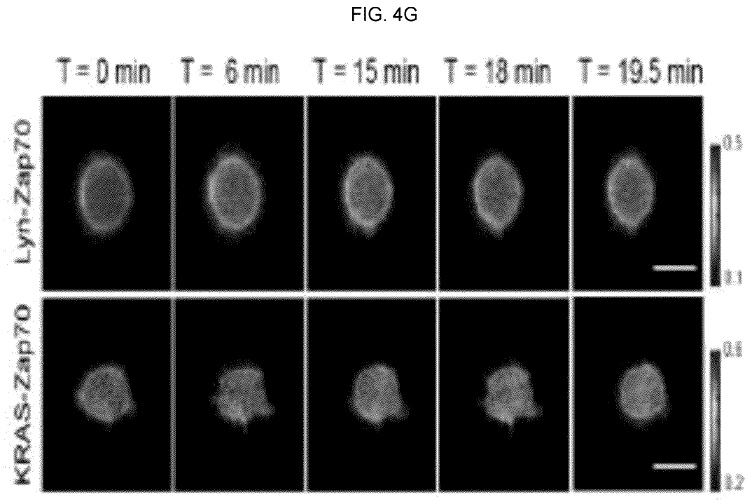

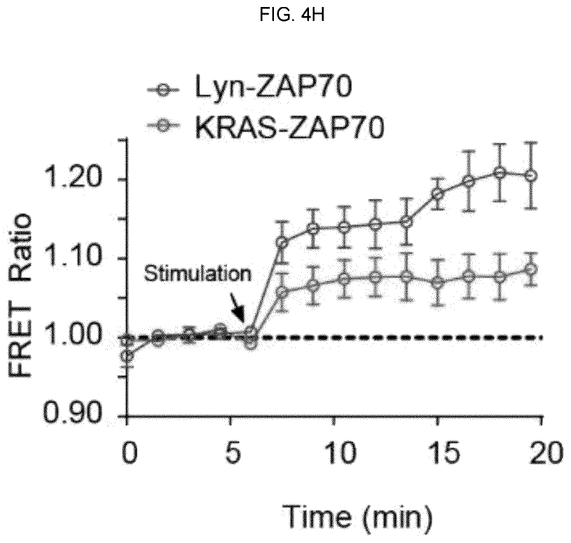

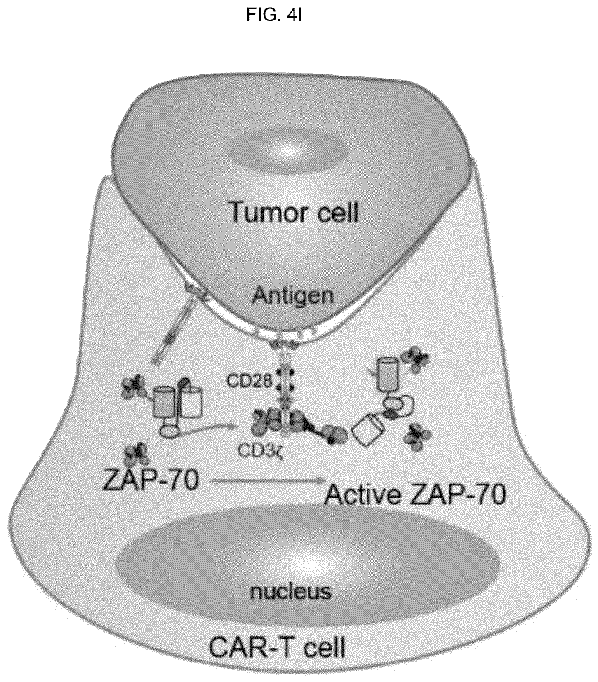

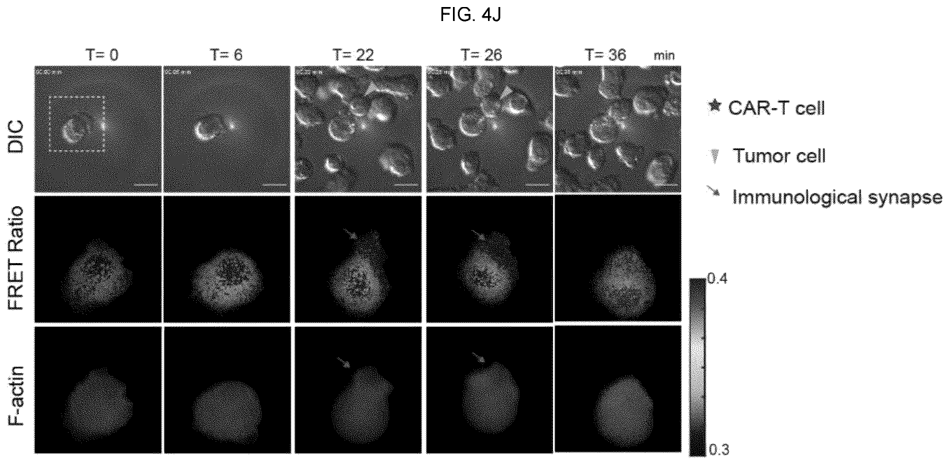

Previous reports suggest that the amino acid residues located downstream of tyrosine may be more important in determining the substrate response to kinases and the binding of phosphorylated peptides toward SH2 domains^{29, 30}. Hence, we first examined the Lib2 variants targeting residues downstream of the consensus tyrosine to verify the selection strategy. Among the forty variants of Fyn biosensors enriched in the KAH group, multiple biosensor variants were identified to have significantly improved dynamic changes comparing to the parent Fyn biosensor upon treatment with PP1, an inhibitor of Src family kinases including Fyn (FIG. 2c-e, Supplementary FIG. 2a-b, or FIG. 8A-B). The probability of identifying a better biosensor than the parental biosensor increased significantly from one-dimensional selection (KAH only) to four-dimensional selection after further filtering the variants via KAL, KDH, KDL enrichment criteria, thus verifying the importance of the multiplex selection strategy. The product of Eᵥ(KAH) and Eᵥ(KDL) was also found to be an efficient ranking factor for the desired biosensor candidates, with the success rate of identifying a biosensor better than the parent biosensor increased from 36% to 84% when the product value was raised to be over 2.2 in evaluating the 40 tested clones (FIG. 2F and Supplementary FIG. 2c-d, or FIG. 8C-D). In fact, a selected biosensor with the EKIEGTYHWF (SEQ ID NO: 1) substrate sequence demonstrated a approximately 60% increase in sensitivity to PP1 treatment in HEK cells, compared to the parental biosensor (FIG. 2C-E). With a similar filtering and selection approach, the success rate for Lib1 to identify better candidates than the parental biosensor was significantly lower than that of Lib2 (Supplementary FIG. 3, or FIG. 9), consistent with previous publications^{29, 30}. Interestingly, the combination of two improved mutants from Lib1 and Lib2 did not further improve the performance of the biosensor (Supplementary FIG. 4, or FIG. 10), potentially due to the uncooperative effect of amino acids up- and down-stream of tyrosine in the substrate in response to recognition by kinase and SH2 domain. Our findings indicate that the FRET-Seq platform combining FRET-based FACS screening with sorting and NGS can directly optimize the Fyn FRET biosensor in mammalian cells.

### Extending the Platform for ZAP70 Biosensor Optimization

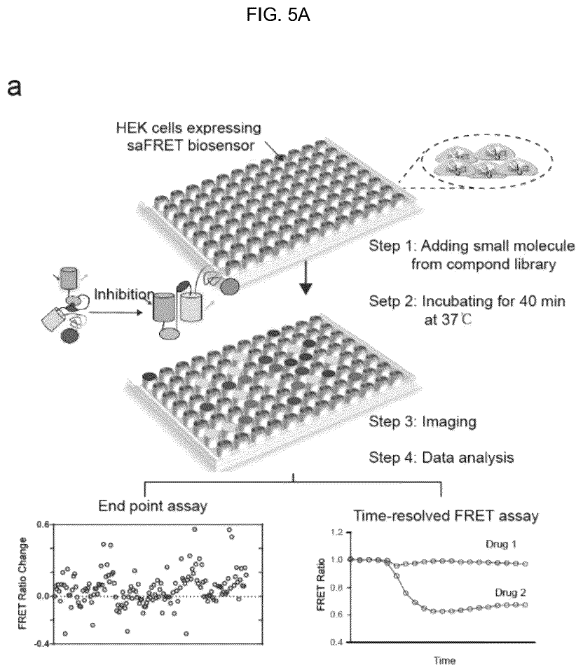

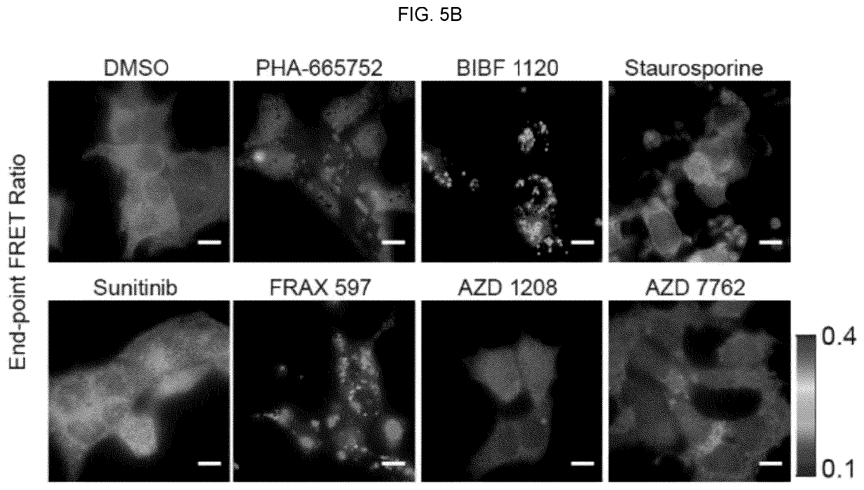

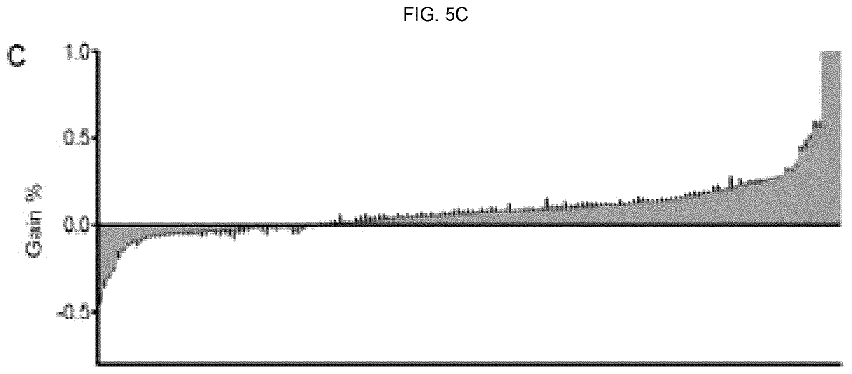

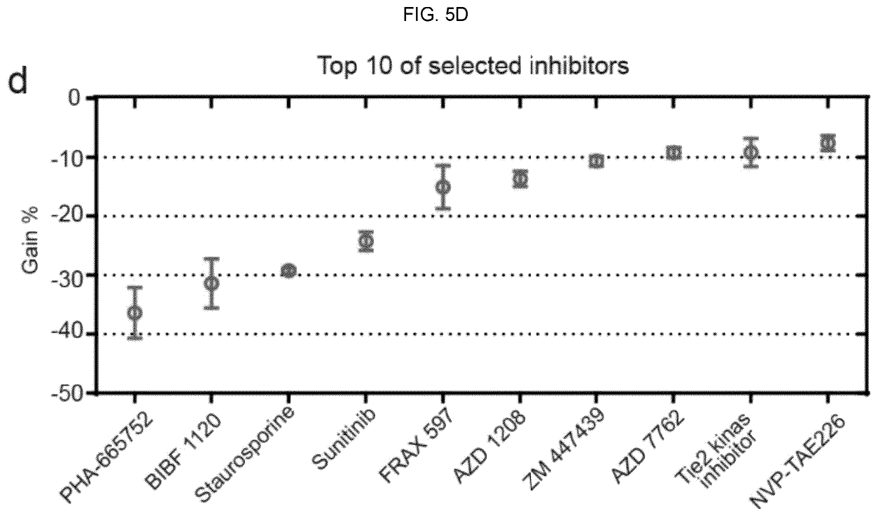

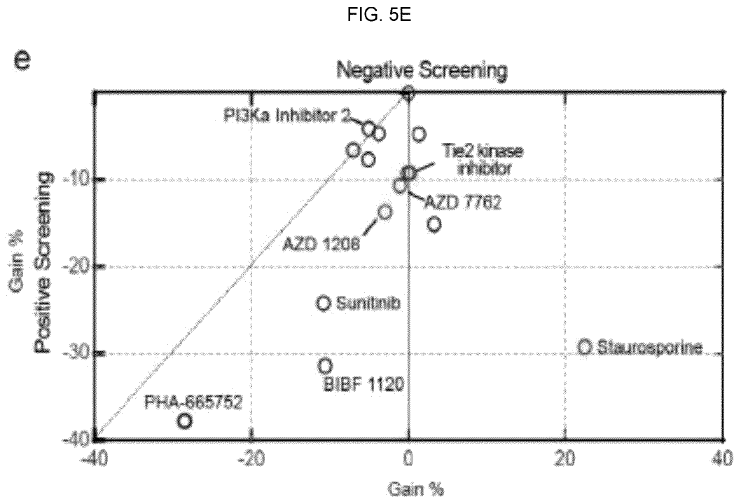

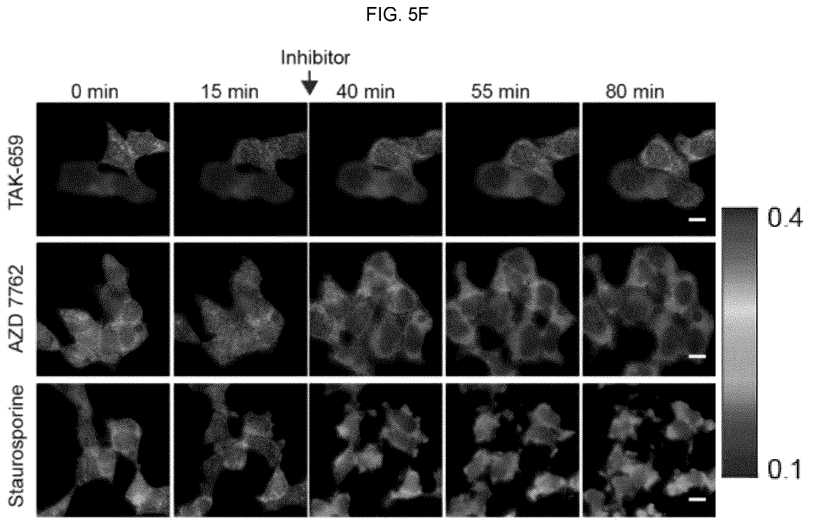

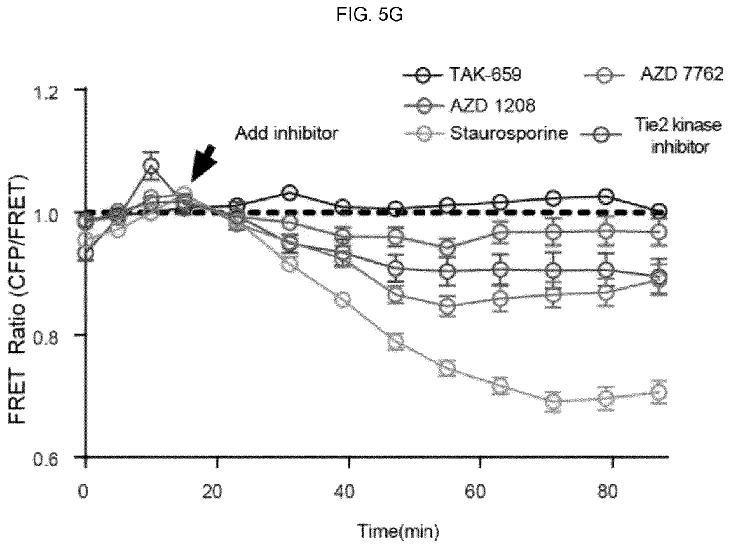

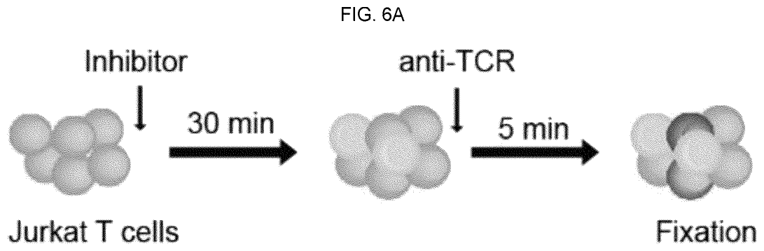

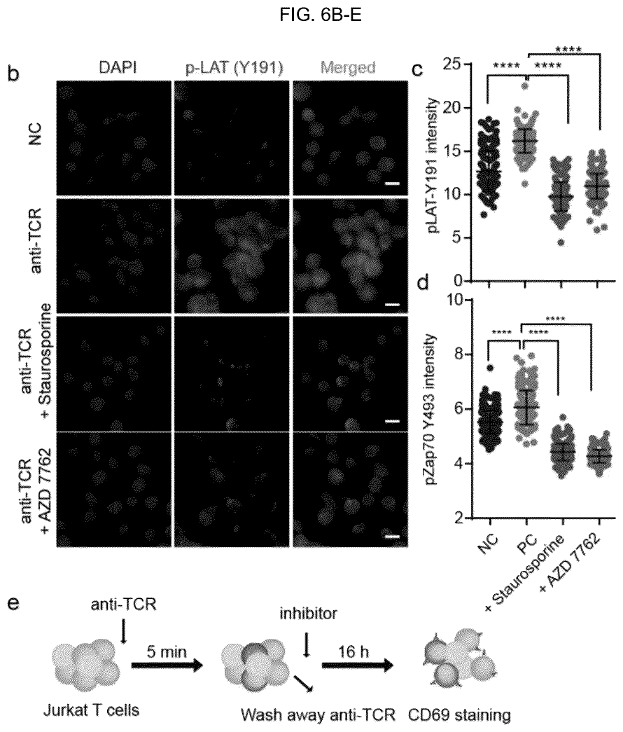

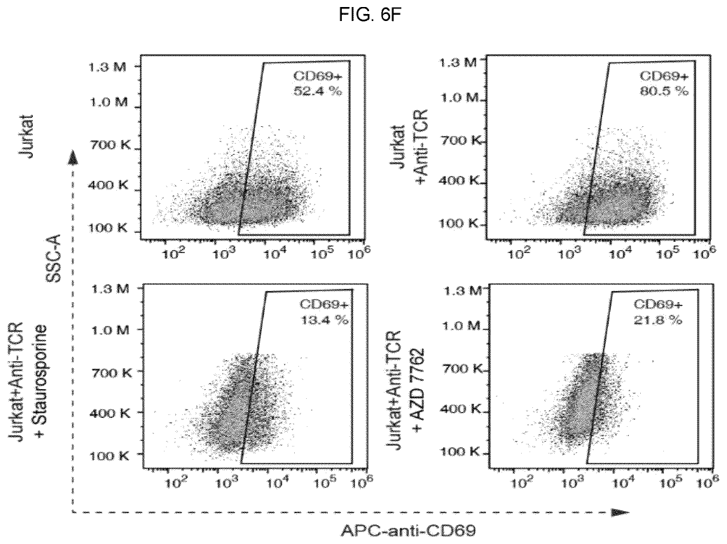

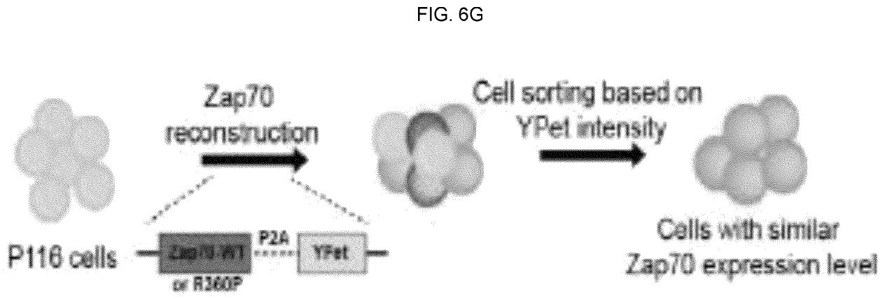

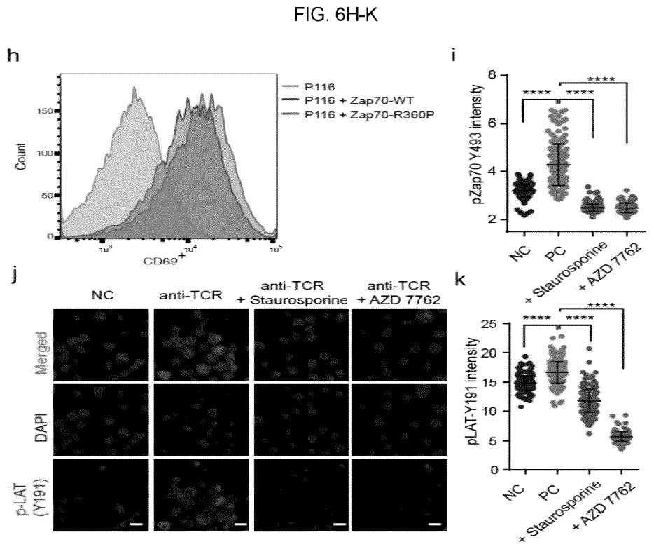

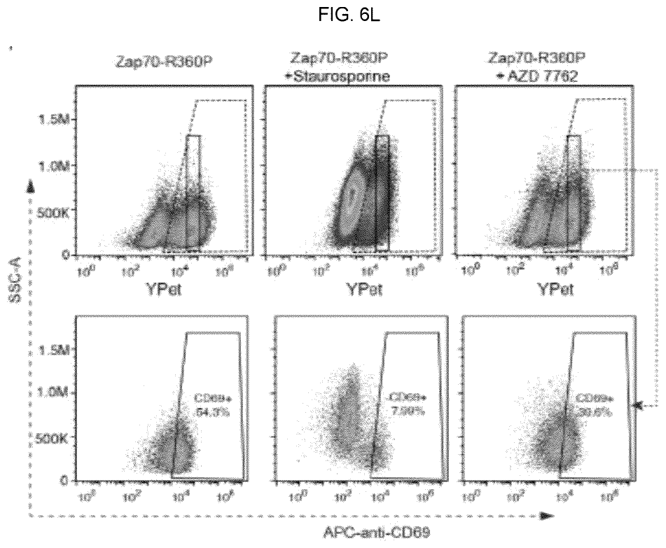

To extend FRET-seq as a general platform to optimize different kinase FRET biosensors in mammalian cells, we further applied this technology to improve the ZAP70 kinase FRET biosensor. A ZAP70 saFRET biosensor was constructed by fusing ZAP70 kinase domain to a ZAP70 FRET biosensor through the EV linker (FIG. 3a), with the substrate sequences derived from the ZAP70 substrate molecule VAV2³⁶ or LAT^{37, 38} The combination of ZAP70 kinase domain (327-619) and a substrate from LATY191 (SREYVNVSGEL (SEQ ID NO:107)) 15 showed an efficient phosphorylation level of the saFRET biosensor (Supplementary FIG. 5a, or FIG. 11A). The high performance of this combination was further verified by live-cell imaging, in which the saFRET biosensor specifically responded to TAK-659 (25 μM), a moderate inhibitor of ZAP70 kinase³⁹ (FIG. 3b,c, Supplementary FIG. 5b, or FIG. 11B), but not to PP2, a Src family kinase inhibitor⁴⁰ (Supplementary FIG. 5c,d, or FIG. 11C-D). Similar to the Fyn saFRET biosensor, the FRET change of ZAP70 saFRET biosensor was dominated by the active kinase domain, as evidenced by the observations that the TAK-659-induced dynamic changes (FIG. 3 b,c) and phosphorylation (Supplementary FIG. 5a, or FIG. 11A) were abolished when the kinase domain was replaced by its kinase-dead version (K369A)⁴¹. Hence, we selected the substrate LATY191 and the kinase domain 327-619 for generating the template of ZAP70 saFRET biosensor to develop substrate mutant libraries, including Library 1 (Lib1: −1, −2, −3, Y) and Library 2 (Lib2: Y, +1, +2, +3) (Supplementary FIG. 6, or FIG. 12).

Using the established platform for library screening and sequencing, the ZAP70 biosensor candidates selected via the four-dimensional plot were further ranked by the product of Eᵥ (KAH) and Eᵥ (KDL) (FIG. 3D-E). The screening of Lib1 did not lead to a better biosensor (Supplementary FIG. 7, or FIG. 13), consistent with the finding of the Fyn biosensor library. In contrast, six of the selected variants from Lib2 showed significantly higher FRET ratio changes than the parental biosensor upon TAK-659 treatment (FIG. 3F-G). The dynamic range of FRET ratio of the biosensors containing SREYACISGEL (SEQ ID NO:113) or SREYYDMSGEL (SEQ ID NO:114) increased approximately 50% comparing to the parental one, when transiently expressed in HEK cells (FIG. 3G-H). As such, our FRET-seq platform can serve as a general high-throughput approach to engineer FRET biosensor from libraries and develop sensitive kinase biosensors.

Visualizing T Cell Signaling with the Improved Biosensors

To examine the functionality of our improved biosensors, we removed the kinase domain and applied the ZAP70 biosensor to monitor T cell activation. Our ZAP70 biosensors demonstrated large FRET ratio changes in Jurkat T cells, but not in their ZAP70-deficient derivative, P116 cells, when stimulated with CD3/CD28-antibody clusters to activate the T cell receptors (TCRs)-ZAP70 signaling pathway⁴² (FIG. 4a-e and supplementary FIG. 8a-c, or FIG. 14A-C). The selected variant SREYACISGEL (SEQ ID NO:113) exhibited more than four-fold increase in sensitivity at 25±10%, comparing to the parental template (SREYVNVSGEL (SEQ ID NO:107)) at 5.8±9% (FIG. 4b-e). These results suggest that our biosensor has improved sensitivity and specificity in reporting ZAP70 kinase activity.

We further applied our improved biosensor (SREYACISGEL (SEQ ID NO: 113)) to visualize the ZAP70 activities in membrane compartments. During T cell activation, TCR becomes phosphorylated by Lck kinase to recruit and activate ZAP70 for the formation of the detergent-resistant microdomains located in lipid rafts⁴³⁻⁴⁵ (FIG. 4f). Our Lyn- and Kras-tagged ZAP70 biosensors were engineered to anchor at the cell membrane, with their Lyn- and Kras-tags targeting rafts and non-raft compartments⁴⁶, respectively (supplementary FIG. 8d, or FIG. 14D). The FRET ratios of the biosensors with Lyn-tag, but not Kras-tag, were significantly elevated upon TCR activation in Jurkat (FIG. 4G-H) or human primary CD4+ T cells (Supplementary FIG. 8e,f, or FIG. 14E-F). This observation suggests that the activation of the ZAP70 kinase upon TCR stimulation mainly occurs in membrane rafts, consistent with prior studies^{44, 45}. The improved biosensor also allowed dynamic imaging of ZAP70 activity during CAR-T/tumor cell engagement (FIG. 4i). Spatially polarized ZAP70 activity was observed to be transiently enriched in the immunological synapse after engagement, but gradually declined during cell detachment and synapse dissolution (FIG. 4J). Hence, our improved biosensors are applicable to visualize the physiological ZAP70 activities in T cells with high spatiotemporal resolutions in both TCR and CAR signaling.

### High Throughput FRET-Based Drug Screening

The saFRET biosensor design enables us to screen small molecule inhibitors of ZAP70 kinase activity in adherent HEK cells, which overcomes the limitation of using suspension cells (Supplementary FIG. 9a, or FIG. 15A) and is compatible with high-throughput drug screening (HTDS) utilizing FRET imaging. Furthermore, HEK cells lack endogenous ZAP70 kinase⁴⁷ and have minimized the heterogeneous background noise of individual cells as in T cells. A stable HEK cell line was first established to express similar copy numbers of the ZAP70 saFRET biosensor (SREYACISGEL (SEQ ID NO:113)) whose average FRET ratio reduction was approximately 25% when treated with TAK695 (25 μM) without noticeable cytotoxicity (Supplementary FIG. 9b-d, or FIG. 15B-D). This sensitivity of the biosensor should allow an image-based, high-throughput platform in 96-well glass-bottom plates capable of both endpoint and quantitative real-time FRET measurements (FIG. 5A), thus overcoming the difficulty attributed to the relatively weak sensitivity of the parent biosensor unsuitable for HTDS assays²⁵ (Supplementary FIG. 5b, or FIG. 11B).

We first screened a 96-member kinase inhibitor library to identify efficient inhibitors of ZAP70 kinase. During the screening, library inhibitors at 10 μM were used as the screening dosage to identify the inhibitors more potent than TAK-659, which was relatively ineffective in suppressing ZAP70 activity at 10 μM (Supplementary FIG. 10a, or FIG. 16A). After 40 minutes of incubation with inhibitors, the cells were imaged, and the FRET ratio changes of individual inhibitors compared to the solvent control were calculated to identify promising candidates after this primary endpoint screening (FIG. 5B-D). We further exploited a control biosensor with a kinase-dead domain (saFRETkd) for counter screening to eliminate false-positive hits due to auto-fluorescence or other non-specific effects⁴⁸. Among the candidates identified, PHA665752, Pi3Kα inhibitor 2, and sunitinib showed a nonspecific reduction of FRET ratio with saFRETkd, which were eliminated from further analysis (FIG. 5e and Supplementary FIG. 10b, or FIG. 16B). Dynamic tracking of the changes in FRET ratio of the remaining biosensor candidates after inhibitor treatment verified the endpoint screening results (FIG. 5f,g). This HTDS assay revealed that Staurosporine, AZD7762, and Tie2 kinase inhibitor from the compound library can effectively inhibit the ZAP70 activity.

### Staurosporine and AZD7762 Inhibit Clinical-Relevant T Cell Activation

We tested the phosphorylation of LAT, a downstream substrate of ZAP70 kinase, and the activation of T cells with the top two identified inhibitors, staurosporine and AZD7762. Significant reductions of phosphorylated LAT (Y191) (FIG. 6A-C) and ZAP70 (FIG. 6d and Supplementary FIG. 11, or FIG. 17) were observed after treatments with staurosporine or AZD7762, verifying the accuracy of the biosensor screening results. These two small molecules also suppressed the expression of CD69 (FIG. 6E-F), a surface marker for activated T cells.

To examine the efficacy of staurosporine and AZD7762 in inhibiting pathological T cell activation, we used a disease model where T cell activation is mediated by ZAP70-R360P mutation, a main cause of a severe human autoimmune syndrome⁴. We introduced wild type ZAP70 or the R360P-mutant of ZAP70 into ZAP70-deficient P116 T cells (FIG. 6G). ZAP70-R360P led to enhanced T cell auto-activation compared to wild type ZAP70 (FIG. 6H). Staurosporine or AZD7762 significantly inhibited the phosphorylation of ZAP70 and LAT (Y191) (FIG. 6I-K, and Supplementary FIG. 11, or FIG. 17), and the subsequently activation of T cells marked by CD69 (FIG. 6L). Hence, staurosporine or AZD7762 may be applicable to mitigate ZAP70R360P-mediated autoimmune disease. As such, the inhibitors identified through the saFRET-HTDS assay can efficiently inhibit the ZAP70 kinase signaling pathway and its subsequent T cell activation, which can have therapeutic potentials in treating diseases involving abnormal ZAP70 kinase or T cell activation.

### Discussion

We have developed the FRET-Seq platform to improve FRET biosensors directly in mammalian cells in a high-throughput manner. This strategy combines several techniques to systematically and efficiently develop FRET biosensors. First, high-throughput FACS screening and sorting based on FRET allow the selection of improved variants from comprehensive libraries in large scale, and these can then be identified by the integration of NGS and analysis. Second, the saFRET design can overcome difficulties in mammalian-cell library screening caused by the heterogenic kinase activities from individual cells. Third, the counter-sorting strategy incorporating a kinase-active or kinase-dead domain in biosensor variants promotes the biosensor specificity during the screening process. The FRET-Seq platform can be readily extended to screen more diverse substrate libraries, or integrated with in silico simulation to optimize other important components of the FRET biosensors, such as the linker and/or SH2 domain for phosphorylated tyrosine³⁸, by identifying the hot spots for mutagenesis to increase the success rate of library screening⁴⁹. While Fyn and ZAP70 kinase biosensors were chosen as the primary targets in this study to provide the proof-of-concept and verification for our approach, the FRET-Seq platform is generalizable to optimize a broad range of other fluorescent biosensors, particularly those for detecting enzyme-based posttranslational modifications. These improved biosensors should enable us to monitor signaling events in single live cells with unprecedented sensitivity and specificity. It is of note that the performance of FRET biosensor is determined by multifactor (for example, kinase selectivity of substrates, the orientation and affinity of the substrate binding to SH2 domain), the substrates that are optimal for biosensor may not be the same as the ones preferred only by the kinases^{50, 51}.

The high-throughput FRET imaging platform using the improved saFRET biosensors allowed the HTDS of efficient and specific small molecules in live mammalian cells for therapeutic purposes²¹, overcoming issues in conventional assays related to cell permeability and cytotoxicity^{52, 53, 54}. The saFRET biosensor design also enables the stabilization of FRET signals in adherent cells, which is crucial for HTDS assay to screen inhibitors targeting kinases that are mainly expressed in suspension cells, for example, ZAP70. Since ZAP70 kinase is crucial for T-cell functions, but can be compensated by Syk in innate immunity⁵⁵, specific inhibitors of ZAP70 kinase (but not targeting Syk) should not cause perturbation of innate immunity and hence can have a high selectivity in targeting T-cell related diseases, for example, controlling allograft rejection and autoimmune diseases such as rheumatoid arthritis, and multiple sclerosis¹⁵. Furthermore, our saFRET biosensor with a ZAP70 kinase-dead domain in HEK cells expressing Syk kinase⁵⁶ had a low basal level and did not show any response after TAK-659 treatment which targets both ZAP70 and Syk (FIG. 3b,c), suggesting that the FRET signals of our saFRET biosensor is unlikely affected by the Syk activities. Thus, the inhibitors identified using this assay should be selective for ZAP70 kinase. This is significant as the inhibition of both ZAP70 and Syk can have devastating effect on immunity and coagulation. Using high content screening platforms equipped with fully automated cellular imaging apparatus and analysis algorithms, the saFRET-HTDS system should also be readily applicable to screen large-scale compound libraries for novel drug discovery or repurposing of FDA approved drugs⁵⁷. These large-scale libraries can also allow counter-screening against other kinases (for example, Syk, Lck) incorporating into our saFRET biosensors to further screen and identify ZAP70 inhibitors with high selectivity.

### Example 1 Figure Legends

### FIG. 1, Example 1. Construction and Validation of saFRET Biosensors

a, Schematics of mammalian cell biosensor library development, screening and sequencing in mammalian cells.
b, Domain structure and activation mechanism of a saFRET biosensor with a fused kinase domain.
c-d, Representative images (c) and time courses (d) of Fyn-saFRET biosensor with active kinase domain (KA) (n=98) or kinase-dead domain (KD) (n=69) before and after PP1 treatment. Error bars, mean±SD. Scale bars, 10 μm. The color bar indicates enhanced cyan fluorescent protein (CFP) (ECFP)/FRET emission ratio, with hot and cold colors representing the high and low ratios, respectively.

### FIG. 2, Example 1. Identification of Biosensors by NGS and Sequence-Function Analysis

a, Workflow of sequencing data analysis.
b, Four-dimensional (4D) plot of the enrichment ratios (Eᵥ) of substrate sequences from different sorting groups. The enrichment ratios in KAH group (Eᵥ(KAH)) are color-coded, whereas Eᵥ(KAL), Eᵥ(KDH) and Eᵥ(KDL) are plotted along with the three-dimensional coordinates. The selected substrate sequences are highlighted with colors represented by the values of their Eᵥ(KAH).
c, Representative time-lapse images of the parental (WT) and improved biosensor (EKIEGTYHWF) (SEQ ID NO: 1) before and after PP1 treatment. Scale bars, 10 μm. The color bar indicates ECFP/FRET ratio, with hot and cold colors representing the high and low ratios, respectively.
D, The quantified dynamic changes of biosensor variants (EKIEGTYXXX) (SEQ ID NO: 2) upon PP1 treatment (n≥20 for each group). Error bars, mean±SD.
e. Time courses of normalized ECFP/FRET ratio of the biosensor variants, with that of the parental biosensor labeled in black (n≥20 for each group). Error bars, Mean±SEM.
f, Mapping of verified substrates in the scatter plot of the enrichment ratios. The dynamic ranges of biosensors were found to have a positive correlation with the product of Eᵥ(KAH) and Eᵥ(KDL). The percentage indicates the success rate of identify better biosensors in different product groups (above or below the contour line of 2.2). Red and blue dots represent biosensor variants with better and worse performance than the parent biosensor, respectively. The red dotted lines represent the contour lines of product of Eᵥ(KAH) and Eᵥ(KDL).
e, Quantification of the basal FRET ratio of Fyn-saFRET biosensor with KA (n=98) or KD domain (n=69). (Unpaired two-tailed Student's t-test, ****P<0.0001). Error bars, mean±SD.
f, Western blot analysis of the biosensor phosphorylations.
g, Modularized template for the library generation of biosensor variants. The bottom panel illustrates the PCR product of substrate variants using the NNK primer.

### FIG. 3, Example 1. Development and Optimization of ZAP70 FRET Biosensor

a, Design of the self-activating ZAP70 FRET biosensor as the screening template.
b-c, Representative images (b) and time courses (c) of FRET ratios of the ZAP70 saFRET biosensor with Active-(KA, n=31) or Dead-(KD, n=19) kinase domain, before and after TAK-659 treatment. Error bars, mean±SD. Scale bars, 10 μm.
d, The 4D plot of the four enrichment ratios (Eᵥ) of substrate sequences. The enrichment ratios in KAH group (Eᵥ(KAH)) was color-coded, whereas Ev(KAL), Ev(KDH) and Ev(KDL) are plotted along the three dimensional coordinates. The selected substrate sequences are highlighted with colors represented by the values of their Ev(KAH).
e, Scatter plot of the substrates. The ZAP70 saFRET biosensors with the top 10 highest products of Ev(KAH) and Ev(KDL) were labeled in red (better biosensors) or blue (worse biosensors).
f, Time-lapse images of the parental (WT) and two selected saFRET biosensors after TAK-659 treatment. Scale bars, 10 μm.
g, Percentage changes of saFRET biosensor variants after TAK-659 treatment (n≥15 for each group). Error bars, mean±SD.
h, Time courses of FRET ratio of the selected saFRET biosensor variants (SREYXXXSGEL (SEQ ID NO:43)), with that of the parental biosensor (WT) marked in black (n is greater than or equal to (≥) 15 for each group). Error bars, Mean±SEM.
b,e, The color bar indicates ECFP/FRET ratio, with hot and cold colors representing the high and low ratios, respectively.

### FIG. 4, Example 1. The Sensitivity and Specificity of the ZAP70 FRET Biosensor in Human T Cell

a, Working mechanism of the ZAP70 biosensor in reporting TCR signaling.
b-e, Time-lapse ECFP/FRET ratio (FRET ratio) images (b,d) and time courses (c,e) of improved (b,c) or parental (WT) (d,e) biosensors before and after TCR activation induced by CD3/CD28 antibody stimulation (n is greater than or equal to (≥) 37 for each group). Error bars, mean±SD. Scale bars, 10 μm.
f, Schematics of membrane-bound biosensors which target different membrane compartments. Lyn- and Kras-ZAP70 biosensors target the lipid rafts or non-raft regions, respectively.
g, Time-lapse FRET ratio images of ZAP70 activities in different membrane compartments after TCR activation. Scale bars, 10 μm.
h, Time courses of ECFP/FRET ratio of ZAP70 biosensor in different membrane compartments before and after CD3/CD28 antibody stimulation. (N=8 and 9 in each group) Error bars, mean±SEM.
i, Schematics of CD19-CAR Jurkat T cell engaging with a CD19⁺ tumor Toledo cell.
j Time-lapse FRET ratio images of CAR-T cell expressing the improved ZAP70 biosensor before and after the engagement with a target tumor Toledo cell. Scale bars, 10 μm.
The color bar in b, d, g, j indicates FRET ratio (ECFP/FRET), with hot and cold colors representing the high and low ratios, respectively.

### FIG. 5, Example 1: High-Throughput Drug Screening Platform Using saFRET Biosensor

a, Schematics of the high throughput drug screening platform. First, the cells cultured in 96-well glass bottom plate were treated either with DMSO or inhibitors from the kinase inhibitor library. After 40 minutes of incubation, the cells were imaged, and the FRET ratio change compared to the control cell was calculated. This platform can also allow dynamic tracking of the FRET ratio change after inhibitor treatment in single cells.
b, FRET-Ratio images of the cells with different inhibitors. Scale bars, 10 μm.
c, Summary of screening results. Some of the inhibitors have shown high efficiency in inhibiting ZAP70 kinase.
d, Top 10 selected inhibitors (n is greater than or equal to (_) 25 for each group). Error bars, mean±SD.
e, Counter screening using a mutant biosensor with a kinase-dead domain to subtract the noise engendered from non-specific fluorescence. The Scatter plot illustrates the FRET ratio changes in the positive and negative screenings using the saFRET biosensor fused with an active kinase or a kinase-dead domain, respectively.
f, FRET ratio images of live-cell imaging with different inhibitors. The TAK-659 (10 μM) was used as the negative control, which cannot sufficiently inhibit the ZAP70 kinase. Scale bars, 10 μm.
g, Time courses of the FRET ratio before and after inhibitor treatment (n is greater than or equal to (≥) 8 for each group). Error bars, mean±SEM.

### FIG. 6, Example 1: Inhibition of T Cell Activation by the HTDS-Identified ZAP70 Inhibitors: Staurosporine and AZD7762

a, Experimental scheme and timeline for experiments in b-d. The Jurkat T cells were pre-treated with inhibitors for 30 minutes before anti-TCR stimulation by anti-CD3/CD28 antibodies for 5 minutes.
b, Immunostaining images of pLAT (Y191) in Jurkat T cells with different inhibitor pre-treatments. Scale bars, 10 μm.
c, Quantification of pLAT (Y191) intensity of single cells in different groups. (n>150 for each group, One-way ANOVA, ****P<0.0001). Error bars, mean±SD.
d, Quantification of pZAP70 (Y493) intensity of single cells in different groups. (n>200 for each group, One-way ANOVA, ****P<0.0001). Error bars, mean±SD.
e, Experimental scheme and timeline for CD69 staining experiment.
f, Flow-cytometry analysis of CD69 expression in T cells after anti-TCR stimulation, with different inhibitor pre-treatments.
g, Experimental scheme and timeline of P116 cells reconstituted with ZAP70. Full length ZAP70-WT or R360P were expressed with YPet via a cleavable P2A linker. P116 cells with similar ZAP70-WT or ZAP70-R360P expressions were sorted and isolated for further analysis based on YPet intensity.
h, CD69 expression in P116 cells with or without the expression of ZAP70 (WT) and its mutant (R360P).
i, Quantification of pZAP70 (Y493) intensity of single cells in different P116 groups. (n>100 for each group, One-way ANOVA, ****P<0.0001). Error bars, mean±SD.
j, Images of pLAT (Y191) in P116-ZAP70 R360P cells with different inhibitor pre-treatments. Scale bars, 10 μm.
k, Quantification of pLAT (Y191) intensity of single cells in P116-ZAP70 R360P cells with different inhibitor pre-treatment. (n>150 for each group, One-way ANOVA, ****P<0.0001). Error bars, mean±SD.
l, Flow-cytometric analysis of CD69 expression in P116-ZAP70-R360P cells with different inhibitor pre-treatment. ZAP70-WT or ZAP70-R360P expression levels were indicated by YPet intensity.

### FIG. 7A-D, or Supplementary FIG. 1: Mammalian Cell Library Screening by FACS

a, Sanger sequencing results showing random mutagenesis in the mutation region of the substrate peptide where EKIXXXYGVV (SEQ ID NO:54) represents library 1 (Lib1) with active (KA) or dead kinase (KD), and EKIEGTYXXX (SEQ ID NO:2) represents library 2 (Lib2) with active (KA) or dead kinase (KD). TAC encodes for tyrosine.
b, Schematic of mammalian cell library screening by FACS. By using FACS, we can analyze the ECFP/FRET ratio of the FRET biosensor variants expressed in single cells.
c, Different control groups in FACS experiment. From left to right: only ECFP-expressing cells, only YPet-expressing cells, co-expression of ECFP- and YPet-expressing cells, mixture of only ECFP- or YPet-expressing cells, cells with KD FRET biosensor, cells with KA FRET biosensor. The top panel shows the relation between YPet intensity (y-axis) and ECFP intensity (x-axis); The bottom panel shows the relation between FRET intensity (y-axis) and ECFP intensity (x-axis).
d, Illustration of FACS experiment. After gate setting using the control biosensors in c, we analyzed and sorted the cells from different libraries. After single-cell gating, the cells with medium expression of FRET biosensor (as represented by YPet expression intensity) were gated and divided into High and Low ECFP/FRET ratio groups. Based on the ECFP/FRET ratio shown in the histogram plot, we can successfully separate the cells with different ratios (CFP/FRET).

### FIG. 8A-D, or Supplementary FIG. 2. The Positive Correlation of Biosensors Between the Improved Performance and the Product of Eᵥ(KAH) and Eᵥ(KDL)

a, The desired biosensors identified were verified to be not enriched in either KAL or KDH group.
b, Quantification of the dynamic ECFP/FRET ratio of the worse biosensor variants tested. The time course of ECFP/FRET ratio of the wild type biosensor before and after PP1 treatment was labeled as a black line (n is greater than or equal to (≥) 15 for each group). Error bars, Mean±SEM.
c. The relation between the dynamic range (%) and the product of Eᵥ(KAH) and Eᵥ(KDL). The dash lines represent the dynamic change (across y-axis) and the value of Eᵥ(KAH)×Eᵥ(KDL) (across x-axis) of wild-type biosensor.
d. The biosensors with different levels of Eᵥ(KAH)×Eᵥ(KDL) were divided into four groups and their time courses accordingly colored with red, pink, light blue, and blue.

### FIG. 9A-G, or Supplementary FIG. 3. The Improvement of Fyn FRET Biosensor Via Lib1

a, The 4D plot of the enrichment ratio of substrates in different groups for Lib1 (xxxY), in which the amino acid residues before the consensus tyrosine were mutated.
The enrichment ratio of the biosensors in the KAH group was color-coded. The substrates satisfying all four criteria were highlighted in color.
b, Representative time-lapse images of the parental biosensor and one of the selected biosensors after PP1 treatment. Scale bars, 10 μm. The color bar represents the ECFP/FRET ratio, with hot and cold colors representing the high and low ratios, respectively.
c, Quantification of the FRET dynamic change (%) of selected biosensor variants upon PP1 treatment (n is greater than or equal to (≥) 15 in each group). Error bars, mean±SD.
d-e, Quantification of the normalized dynamic ECFP/FRET ratio of the better (d) and worse (e) biosensor variants that have been tested. FRET ratio change of the parental biosensor was marked in black line (n is greater than or equal to (≥) 15 in each group). Error bars, Mean±SEM.
f-g, Scatter plot of the enrichment ratio of biosensor variants. Red and blue dots represent biosensor variants with better and worse performance than the parental biosensor, respectively.

### FIG. 10A-B, or Supplementary FIG. 4. The Combination of Two Improved Mutants from Lib 1 and Lib2

a. Comparison of the biosensors with combined sequences from both Lib 1 and Lib 2 vs their parental improved biosensors from either Lib 1 or Lib2. Star indicates the biosensors with combined substrate sequences (left columns). The middle columns are improved biosensors from Lib1 and the right columns from Lib2. The dashed line indicates the mean FRET change of original WT (EGTYGVV) (SEQ ID NO:55) biosensor. (N is greater than or equal to (≥) 15 for each group, One-way ANOVA, ****P<0.0001, **P=0.0038, *P=0.0291, NS═Not significant).
b. Time courses of the ECFP/FRET ratio signals of the combined biosensors after PP1 treatment. Error bars, Mean±SD.

### FIG. 11A-D, or Supplementary FIG. 5. Examining Kinase Domains and Substrates for ZAP70 saFRET Biosensor

a, The effect of kinase domain on the biosensor phosphorylation. Kinase domain 1: ZAP70 327-619; and Kinase domain 2: ZAP70 327-601.
b, Quantification of the dynamic ECFP/FRET ratio changes of ZAP70 saFRET biosensors with different substrates and kinase domain, upon the treatment by TAK-659 (25 μM, n1 is greater than or equal to (≥) 6 for each group, black-arrow). Percentage indicates the reduction (red-arrow) of FRET ratio after TAK-659 treatment. Reduction of FRET ratio was observed in kinase dead biosensors with substrates from Vav2 and LATY175. (Paired two-tailed t test, ****P<0.0001, NS, Not significant).
c-d, Representative images (c) and time courses (d) of the ECFP/FRET ratio signals of the ZAP70 saFRET biosensor with different inhibitors. TAK-659 (n=27). PP2, a Src family kinase inhibitor (n=13). Error bars, mean±SEM. Scale bars, 10 μm.

### FIG. 12A-B, or Supplementary FIG. 6. Unbiased Library Generation for ZAP70 Biosensor

a, Sequencing results of library 1 (Lib1) with active (KA) or dead kinase (KD). TAC encodes for tyrosine.
b, Sequencing results of library 2 (Lib2) with active (KA) or dead kinase (KD).

### FIG. 13A-D, or Supplementary FIG. 7. The Mutation of Amino Acid Residues Upstream to the Consensus Tyrosine in the Substrate of the Biosensors

a, The 4D plot of the enrichment ratio of substrates from different groups. The enrichment ratio in the KAH group was color-coded. The substrates satisfying all four criteria were highlighted with color.
b, Scatter plot of biosensors with different substrates. The biosensor variants with the top 10 products of Eᵥ(KAH) and Eᵥ(KDL) from Lib1 were labeled in Red (better biosensors than the parental biosensor) or Blue (worse biosensors than the parental biosensor).
c, Quantification of the dynamic change of biosensor variants upon PP1 treatment (n is greater than or equal to (≥) 15 for each group). Error bars, mean±SD.
d, Quantification of the normalized dynamic ECFP/FRET ratio of the selected biosensor variants. FRET ratio change of the parental biosensor was marked in black line (n is greater than or equal to (≥) 15 for each group). Error bars, Mean±SEM.

### FIG. 14A-F, or Supplementary FIG. 8. Verification of the Improved Biosensors in Primary Human CD4+ T Cells

a, Dynamic ranges of the ZAP70 biosensors with different substrates. SREYVNV (SEQ ID NO:53) represents the parental biosensor (n is greater than or equal to (≥) 37 for each group). Jurkat, human T cell line. P116, ZAP70^{-/-} cell line derived from Jurkat cells. (Unpaired two-tailed Student's t-test, ****P<0.0001, NS p=0.446).
b,c, Time courses (b) and time-lapse images (c) and of the SREYYDM (SEQ ID NO: 45) biosensor before and after TCR activation induced by CD3/CD28 antibody stimulation (n is greater than or equal to (≥) 37 for each group). Error bars, mean±SD. Scale bars, 10 μm.
d, The design of the membrane-bound ZAP70 FRET biosensors and their membrane localization in HEK cells.
e, Representative time-lapse images of ZAP70 activity change in different membrane compartments after TCR activation in primary human T cells. Scale bars, 10 μm. The color bar indicates ECFP/FRET intensity ratio, with hot and cold colors representing the high and low ratios, respectively.
f, Time courses of normalized ECFP/FRET ratio of ZAP70 FRET biosensor in different membrane compartments (N=22 in each group). Error bars, mean±SEM.

### FIG. 15A-D, or Supplementary FIG. 9. Stable HEK 293T Cell Line with ZAP70 saFRET Biosensor for HTDS Assay Targeting ZAP70 Kinase

a. Scheme illustrates the advantage of imaging adherent cells compared to suspension cells in general imaging platforms. Suspension cells, such as immune cells, float freely in media, and the focus or the observation field can easily become lost over time, especially at high magnification scale during imaging.
b, Cell sorting of the stable HEK293T cell line with a similar expression level of ZAP70 saFRET biosensor. These sorted cells are used for HTDS assay.
c, The isolated stable HEK cell line expressing ZAP70 saFRET biosensor demonstrated approximately 25% change after a high-dose 25 μM TAK659 treatment (n=40 for each group, unpaired two-tailed Student's t test, ****P<0.0001).
d, Representative ECFP/FRET ratio images of ZAP70 saFRET biosensor after 25 μM TAK659 treatment.

### FIG. 16A-B, or Supplementary FIG. 10. HTDS Using ZAP70 saFRET Biosensors

a, Concentration-dependent response of saFRET biosensor to TAK-659. The 10 μM TAK659 treatment could not reduce the FRET ratio significantly. (n>20 for each group, unpaired two-tailed Student's t test, ****P<0.0001). Error bars, mean±SD.
b, Top panel: The design of the ZAP70 biosensor with kinase-dead domain (saFRETkd). Bottom panel: The FRET ratio changes of a saFRET biosensor with kinase-dead domain in counter screening. Small molecules which have non-specific effects on FRET signals are eliminated in this step. n is greater than or equal to (≥) 10 for each group, Error bars, mean±SD.

### FIG. 17A-B, or Supplementary FIG. 11. Staurosporine and AZD7762 are Potent Inhibitors of ZAP70 Signaling Pathway

a, Representative images of pZAP70 (Y493) in Jurkat T cells with different treatment. Scale bars, 10 μm.
b, Representative images of pZAP70 (Y493) in P116-ZAP70-R360P cells with different treatment. Scale bars, 10 μm.

### FIG. 18A-B, or Supplementary FIG. 12. Uncropped Western Blot Images

Dash line indicates the cropped regions in FIG. 1 (a) and Supplementary FIG. 3 (b), respectively.

### Example 2: Making and Using Exemplary Self-Activating FRET (saFRET) Biosensors

In one platform, firstly we developed a saFRET by linking an active kinase domain to a conventional FRET biosensor, then we utilized the directed evolution approach to optimize the substrate sequence in the sensing unit for the tyrosine kinases (FIG. 1, Step 1). The optimized biosensor could be used for high throughput drug screening (FIG. 1, Step 2).

For each kinase, we make saFRET biosensors by ligating a corresponding kinase domain to the FRET biosensors, as illustrated in FIG. 19.

A biosensor library with different substrate sequences is generated by site-saturated mutagenesis and introduced into HEK cells, with the substrate variants being phosphorylated by the intramolecular kinase domain. The phosphorylation-mediated FRET signals from every single cell expressing biosensors is then screened by FACS to select cells hosting biosensors with the biggest FRET changes. mRNAs from these selected cells are isolated, and the substrate sequences in biosensors amplified for NGS. The substrate sequences with the highest frequency in NGS results are identified as favorable substrates of the corresponding kinase and binding partners of the intramolecular Src Homology 2 (SH2) domain upon phosphorylation. We then verify the sensitivity/specificity of the biosensors based on these new substrates in live cells, as illustrates in FIG. 19.

We have conducted experiments to establish the directed evolution platform for saFRET biosensor optimization using Fyn biosensor for Fyn kinase and verified the universality of the system using Zap70 biosensor for Zap70 kinase and developed the high throughput screening platform using the optimized FRET biosensor.

Construction of saFRET Biosensor as the Optimization Template.

In order to test the feasibility of the platform and establish the selection criteria for saFRET biosensor optimization, we chose the Fyn kinase biosensor as the starting template. To construct the saFRET biosensor and identify the enhanced cyan fluorescent protein (CFP) (ECFP)/YPet emission ratio (FRET ratio) change of biosensor variants in HEK293 cells with low kinase background, we further engineered the conventional biosensor and fused it with a kinase-active (KA) domain to allow for intramolecular interaction between the kinase domain and the substrate peptide, as schematically illustrated in FIG. 20A, 20B.

The gene template for the protein tyrosine kinase biosensor was constructed by polymerase chain reaction (PCR) amplification of the complementary DNA of an enhanced CFP (ECFP), LacZ, YPet, EV linker (116 amino acids) (see for example, Komatsu, Mol Biol Cell. 2011 Dec. 1; 22 (23): 4647-4656), and either active or mutated kinase domains. The amplified elements were fused and inserted into the pSin lentiviral transfer vector (pSin-ELYK), between SpeI and EcoRI with T4 ligation (New England Biolabs) where ECFP is at N-terminal, and the kinase domain is at C-terminal. Several restriction sites were introduced, such as two of Esp3I sites at each end of LacZ for replacing to the sensing domain (including SH2 domain and substrate peptide) and XbaI/EcoRI for replacing to different kinase domains. To construct the biosensor, the cDNA of the sensing domain, which was amplified by PCR from the mutated c-Src SH2 domain (C185A) with a sense primer containing an Esp3I and a reverse primer containing the cDNA of a flexible linker (15 amino acids), a substrate peptide, and an Esp3I site, replaced the LacZ domain via the Golden Gate assembly (New England Biolabs). The substrate peptide sequence can be changed by Golden Gate assembly with different PCR products amplified using different reverse primers. The regulation of phosphorylation level in HEK293 cells depends on the interaction between the substrate and the kinase domain. To select the suitable kinase domain for biosensor template, we tested several kinase domains with different lengths for their activity in phosphorylating the substrate in biosensor using western blot. Kinase domains of Fyn kinase ranging from 265-526, 261-526, 261-537 were tested, and kinase domain 265-526 were selected. A K299M mutation was introduced into the Fyn kinase domain to generate the kinase-dead control. The well-established substrate (SREYYVNVSGEL (SEQ ID NO:106)) for Fyn biosensor was chosen for Fyn biosensor (FIG. 20 C-E). In this design, the FRET ratio change was mediated by the kinase domain, and if we replace the active kinase domain with a kinase-dead domain, the phosphorylation of the substrate (FIG. 20C) and the dynamic change of the FRET biosensor after PP2 treatment was disappeared (FIG. 20D and FIG. 20E). The biosensor with selected kinase domain and substrate was used as a starting template to generate biosensor variants.

### Plasmid Library Construction Using Site-Saturated Mutagenesis

Biosensor libraries were created by site-saturated mutagenesis by using NNK degenerate primers (IDT), where N represents an equimolar distribution of A, T, G, and C; K represents an equimolar distribution of T and G; X represents any amino acid. Briefly, the cDNA of the substrate variants was generated by PCR with Q5 DNA polymerase (NEB, Cat. No. M0491) from the c-Src SH2 domain (C185A) with a sense primer containing an Esp3I and a reverse primer containing a flexible linker. NNK codons were included in the antisense primer for the substrate library (FIG. 21A). The PCR condition of annealing temperature was varied from 55-70° C., and thermocycling condition was 20 cycles. After generating the substrate mixture with NNK degenerated reverse primers, the fragments containing cDNA mixture were then extracted from agarose gel, inserted into pSIN-ELYK template vector between Esp3I restriction sites through the Golden Gate assembly (NEB) to generate the biosensor library. The product was purified and concentrated by using DNA Clean and Concentrator Kits (Zymo) and transformed into ELECTROMAX™ DH10B™ Cells (Invitrogen, Catalog number: 18290015) which are electrocompetent E. coli cells offering the highest transformation efficiencies of greater than (>) 1×10¹⁰ CFU/μg plasmid DNA. Then the plasmid libraries were purified with Qiagen HiSpeed Plasmid Maxi kit (Qiagen), and the mutation regions of each library was further verified using Sanger sequencing (FIG. 21B):

### Generation of Mammalian Cell Library for Biosensors

The plasmids of biosensor libraries were introduced into mammalian cells (HEK293T cells from ATCC) through virus infection with low MOI (0.1) to allow a low copy number of plasmids per a single cell. Lentiviruses were produced from Lenti-X 293T cells (Clontech Laboratories, #632180) co-transfected with a pSin containing biosensor variants and the viral packaging plasmids pCMV-A8.9 and pCMV-VSVG using the PROFECTION MAMMALIAN TRANSFECTION SYSTEM™ (Promega, Cat. No. E1200). Viral medium/supernatant was collected 48 h after transfection, filtered with 0.45 μm filter (Sigma-Millipore), and concentrated using PEG-it virus precipitation solution (System Biosciences, Cat. #LV825A-1). The virus titer was measured by flow cytometry. To generate the mammalian cells library, we then added the concentrated virus with MOI=0.1 directly into HEK293T cells, which were seeded 2×10⁶ cells in 10-cm dish a day before transfection. Cells were then cultured in DMEM medium containing puromycin (2 μg/mL) after 48 h of transfection until screening by fluorescence activated cell sorting (FACS).

### Mammalian Cell Library Screening by FACS

HEK 293T cells containing biosensor variants were screened by FACS (BD FACS Aria II Cell Sorter)-based FRET ratio (ECFP/FRET ratio), which was calculated from the emission of ECFP divided by that of FRET (FIG. 22A). Several controls were used to gate the cell population, including: wild type HEK 293T cells were used to gate for the live cells; cells with ECFP or YPet (a basic, constitutively fluorescent, yellow fluorescent protein) were used to gate for the cells that expressed ECFP only (Ex 405 nm, Em 450/50 nm) or YPet only (Ex 488 nm, Em 545/35 nm); the mixture of cells that express either ECFP or YPet was used as a negative gating of FRET signal (Ex 405 nm, Em 545/35 nm); cells co-transfected with both ECFP and YPet were also used to gate for the intermolecular FRET signal (FIG. 22B). The conformations of intramolecular FRET biosensors can be measured based on the FRET ratio. Thus, cells expressing biosensors fused with active kinase domain (KA) were used to gate for the active conformation of FRET biosensor (high FRET ratio), while, those expressing biosensors with kinase-dead domain (KM) were used to gate for the inactive conformation of FRET biosensor (low FRET ratio) (FIG. 22B). In addition to all gating, only cells expressing the medium intensity of biosensor (by YPet filter; Ex 488 nm, Em 545/35 nm) were selected for sorting to avoid the abnormal expression of biosensors in cells. After gate setting, cells containing biosensor libraries were sorted into high and low FRET ratio, where the median FRET ratio was used as a threshold for sorting. Top 5% of the cells expressing biosensors that had a higher FRET ratio than the median FRET ratio were selected for the high-FRET ratio and vice versa (FIG. 22C). Based on the FRET ratio, the cells were sorted into KAH (High FRET ratio with Active Kinase, KA), KML (Low FRET ratio with Mutated Kinase, KM), KAL (Low FRET ratio with Active Kinase, KA) and KMH (High FRET ratio with Mutated Kinase, KM) groups for each library. At the same time, the cells before sorting in each library were kept as input control. The mRNA of the sorted cells in each group will be collected and generate the sequencing library.

### The Sequence-Function Analysis of Biosensor Library

Substrate libraries were sequenced by Illumina HiSeq 4000 sequencing system. The total RNA of each pool of sorted cells were extracted by RNeasy Mini Kit (Qiagen, Cat #74104). During column purification, the genomic DNA was removed by RQ1 RNase-Free DNase (Promega, Cat #M6101). This allows only RNA that can be encoded to the biosensor proteins to be purified. The RNA was quantified by Nanodrop and gel electrophoresis. The purified total RNA (approximately 500 ng) was used as a template for cDNA synthesis via the SUPERSCRIPT IV™ reverse transcriptase (ThermoFisher Scientific, Cat #18090010) with gene-specific primer. Adaptor sequences with different indexes for Illumina sequencing were added into cDNA by PCR using Q5 DNA polymerase (NEB, Cat #M0491S) with PCR cycles (<16 cycles). Illumina sequencing fusion primers were synthesized from IDT. Take Fyn-Library as an example, the forward primer for sequencing library contains the flow cell binding sequence, sequencing primer sites and constant regions specific to library insert and the reverse primer contains the flow cell binding sequence, sequencing primer sites, adaptor and constant regions specific to library insert. The individual pool of the library was labeled with a different barcode. The amplicon containing all adaptors was confirmed by gel electrophoresis (2% agarose gel) and purified by ZYMOCLEAN™ gel DNA recovery kit (Zymo Research, Cat #D4008). The purified amplicon libraries were sequenced by Sanger sequencing (Genewiz) to verify the success of library preparation and quantified by Qubit prior to being sequenced by ILLUMINA HISEQ4000™ with 50-bp single-end sequencing (for the entire libraries).

### Sequencing Data Analysis and Better Biosensor Predication

Sequencing data were analyzed using the Matlab software. Only the sequences with phred score greater than (>) 20 at all positions, contained the constant regions, TAC region and had the correct length of the insert were selected and converted from nucleotide sequence to amino acid sequence. Then the amino acid sequence from each group was normalized to Counts Per Million (CPM). The sequence with CPM greater than (>) 10 was considered positive and selected for further analysis. Because different libraries had different total sequencing reads, to avoid the bias due to sequencing depths, the frequency of unique sequences was computed by normalizing the variant count in each library to the total number of sequencing reads for that library (FIG. 2A, see Example 1). The frequency of a unique sequence can be compared across different libraries. Since enriching cells containing functional protein variants while depleting nonfunctional variants was achieved by FACS, the change in frequency of each variant from input to selection served as a measure of its function. The frequency for a given variant (Fᵥ) was

$${f}_{v} = \frac{{c}_{v}}{{\sum }_{k = 1}^{n} {c}_{k}}$$

The frequency data was later used to compute variant enrichment ratios, which allowed us to find the fold enrichment of that variant before and after sorting. The enrichment ratio for a given variant (Eᵥ), was

$${E}_{v} = \frac{{f}_{v , sorted}}{{f}_{v , input}} .$$

A better biosensor accurately responding to kinase should be enriched in KAH (High FRET ratio with Active Kinase, KA) and KML (Low FRET ratio with Mutated Kinase, KM) groups, and at the same time should not be enriched in KAL (Low FRET ratio with Active Kinase, KA) or KMH (High FRET ratio with Mutated Kinase, KM) group. Therefore, the variants with Eᵥ above one in KAH and KML group and blow one in KAL and KMH group were further selected and verified. The data for each substrate sequence were visualized in the 4D plot using Matlab software (FIG. 2B, see Example 1):

### Verification of the Selected Biosensor Using Live-Cell Imaging.

Cells expressing the exogenous biosensor proteins were starved with 0.5% FBS DMEM for 12 h before being subjected to PP1 (10 μg/mL) stimulation. Images were taken with a Nikon Eclipse Ti inverted microscope with a cooled charge-coupled device (CCD) camera with a 420DF20 excitation filter, a 450DRLP dichroic mirror, and two emission filters controlled by a filter changer (480DF30 for ECFP and 535DF35 for YPet). The time-lapse fluorescence images were acquired by METAMORPH 7.8™ software (Molecular Devices). The ECFP/FRET ratio images were calculated and visualized with the intensity modified display (IMD) method by Fluocell software (Lu, Kim et al. 2011) (Github http://github.com/lu6007/fluocell). For data presentation, the normalized values were shown to compare the differences among the experimental groups and to minimize the cell-cell heterogeneity. The pre-stimulation baseline for each cell was established by averaging the FRET ratio of each cell before stimulation. We have tested 40 substrates, including the original substrate, to find the criteria for biosensor ranking, and indeed we have successfully identified several better biosensors compared to the original one using live-cell imaging (FIG. 2C-E, see Example 1). Through coupling the sequencing data, we found there was a much higher possibility of identifying a better biosensor when the product of Ev(KAH) and Ev(KML) was larger (FIG. 2F, see Example 1). Therefore, the product of Ev(KAH) and Ev(KML) could be used as ranking criteria.

### Optimization of Zap70 FRET Biosensor Using the Directed Evolution Platform.

We have established the directed evolution platform by using Fyn biosensor as an example. To verify the broad application of this platform, we constructed the saFRET biosensor for Zap70 kinase and optimized the Zap70 biosensor through directed-evolution platform. For ZAP70 kinase saFRET biosensor optimization, similar as Fyn biosensor, we first constructed a screening template by ligating a corresponding kinase domain to the FRET biosensors (see FIG. 1B, and FIG. 3A, Example 1). Zap70 kinase domains of different lengths and substrate peptides from Vav2 (Li, Xiang et al. 2016), LATY175 (Randriamampita, Mouchacca et al. 2008) and LATY191 (Cadra, Gucciardi et al. 2015) were tested. Zap70 biosensor with LATY191 (SREYVNVSGEL (SEQ ID NO:107)) (Cadra, Gucciardi et al. 2015) substrate showed a higher phosphorylation level than biosensors with substrate LATY175 (SCEDYVNVPES (SEQ ID NO:108)) (Randriamampita, Mouchacca et al. 2008) or the Vav2 substrate when coupled with kinase domain 327-619 (FIG. 23A). A significant FRET ratio change can be observed in response to TAK-659, which is a selective inhibitor for Zap70 (Purroy, Abrisqueta et al. 2014) rather than SRC family kinase inhibitor PP2 (FIG. 3B, FIG. 3C, FIG. 3D, Example 1; and FIG. 23B, FIG. 23C). In contrast, the biosensor with mutated kinase domain had no response to TAK659 (FIG. 3B, FIG. 23B, FIG. 23C). All these results indicate that the substrate LATY191 can specifically respond to intramolecular ZAP70 kinase domain. Consequently, Zap70 saFRET biosensor which consisted of LATY191 substrate and kinase 327-619 (both KA and KM) was chosen for generating the Zap70 biosensor substrate libraries (FIG. 3A).

We further optimized the FRET biosensor. Same as the optimization process of Fyn biosensor, a biosensor library with varying substrate sequences was generated by site saturated mutagenesis and introduced into HEK cells, with the substrate variants being phosphorylated by the intramolecular KD. The randomness of the mutation region was verified by the Sanger sequencing that showed equimolar ratios of A, T, C, and G, suggesting the unbiased library generation. Each of these four libraries was introduced to HEK293T cells using lentivirus transduction with MOI=0.1 to generate the mammalian cell library containing Zap70 FRET biosensor variants. Following counter sorting strategy, all libraries were screened based on their FRET ratio and verified by using microscope before further sequenced. To assess sequence-function relationships of the substrate peptide, several parameters were computed and filters were applied (Fowler, Araya et al. 2010). The frequency of unique sequences (variants) was computed by normalizing the variant count in each library to the total number of sequencing reads for that library.

To increase the specificity of the selected substrates, library with mutated kinase were included. A good biosensor specifically respond to ZAP70 activity should be enriched in KAH and KML groups, and at the same time will not be enriched in KAL or KMH group. Therefore, the selected set of variants were further filtered by the following conditions: Ev of KAH and KML should above one while Ev of KAL and KML should blow one (FIG. 6g). The variants which satisfied all conditions were selected (FIG. 3D, Example 1), and their FRET ratio change upon TAK-659 treatment were measured by fluorescence microscope. About 55% of selected variants had shown higher FRET ratio changes than the original wide-type substrates (FIG. 3E, FIG. 3H, FIG. 3G, FIG. 3F). Just after one round of screening, the FRET ratio dynamic range of the optimized biosensors was significantly increased compared to the original one (FIG. 3H, FIG. 3G). At the same time, the sensitivity of the biosensor in response to ZAP70 inhibitor increases dramatically compared to the original substrate (FIG. 3H). Thus, by combining counter sorting and amplicon sequencing, the platform we developed was demonstrated to be a promising high-throughput approach to search for the optimal variants.

Optimized Biosensors have Shown Improved Sensitivity and Specificity in Live T Cells.

To test the specificity and dynamic range of our selected biosensors in immune cells, we transfected the selected biosensors after removing the kinase domain into Jurkat T cells and P116 cells (Jurkat cells with undetectable ZAP70 expression) (FIG. 4A). The cells were stimulated with CD3/CD28-antibody clusters for specifically activating TCR-ZAP70 signaling pathway. The selected biosensors could detect the change of ZAP70 activity with higher sensitivity and dynamic ranges (dynamic range for the biosensors with substrates SREYACI (SEQ ID NO:44) and SREYYDM (SEQ ID NO:45) was 25±10 and 17.6±6 respectively) as indicated by the significant increase of FRET ratio upon CD3/CD28-antibody stimulation while not in P116 cells compared to the original one (dynamic range for the original substrate is 5.8±9) (FIG. 24A), see FIG. 24B-G). These results demonstrate that the optimized version of our biosensors was quite sensitive and specific for further studies in T cell/Tumor cell interaction.

High Throughput Screening Platform Using Optimized saFRET Biosensor

After the optimization of the saFRET biosensor, we established a high throughput screening platform to achieve efficient drug screening for Zap70 kinase. Firstly, we transfected HEK293 cells with optimized saFRET biosensor and established a stable cell line. By using this design, we could screen drugs target the kinase, which only expressed in floating immune cells in adherent HEK293 cells. HEK293 stable cell line was then cultured in 96 glass bottom wells for approximately 24 hours and then should be ready for high throughput screening (see FIG. 5A, Example 1). We tested this concept by screening an in house kinase inhibitor library (Cayman chemical inc.) and screened a 156-member kinase inhibitor library to identify efficient inhibitors of ZAP70 kinase. During the screening, library inhibitors at 10 μM were used as the screening dosage to identify the inhibitors more potent than TAK-659, which was relatively ineffective in suppressing ZAP70 activity at 10 μM. After 40 minutes of incubation with inhibitors, the cells were imaged, and the FRET ratio changes of individual inhibitors compared to the solvent control were calculated to identify promising candidates after this primary endpoint screening (see FIG. 5B-G, Example 1). We further exploited a control biosensor with a mutated-kinase dead domain (saFRETkd) for counter screening to eliminate false-positive hits due to auto-fluorescence or other non-specific effects (Simeonov and Davis 2004). Among the candidates identified, PHA665752, Pi3Kα inhibitor 2, and sunitinib showed a nonspecific reduction of FRET ratio with saFRETkd, which were eliminated from further analysis (FIG. 5E). Dynamic tracking of the changes in FRET ratio of the remaining biosensor candidates after inhibitor treatment verified the endpoint screening results (FIG. 5F-G). This HTDS assay revealed that Staurosporine, AZD7762, and Tie2 kinase inhibitor from the compound library can effectively inhibit the ZAP70 activity. These results demonstrate that our optimized saFRET biosensor could be used in high throughput drug screening to identify potent and specific drugs.

High-Throughput Drug Screening Platform Using saFRET Biosensor:

FIG. 5A: Schematics of the high throughput drug screening platform. First, the cells cultured in 96-well glass bottom plate were treated either with DMSO or inhibitors from the kinase inhibitor library. After 40 minutes of incubation, the cells were imaged, and the FRET ratio change compared to the control cell was calculated. This platform can also allow dynamic tracking of the FRET ratio change after inhibitor treatment in single cells. FIG. 5B, FRET-Ratio images of the cells with different inhibitors. Scale bars, 10 μm. FIG. 5C, Summary of screening results. Some of the inhibitors have shown high efficiency in inhibiting ZAP70 kinase. FIG. 5D, Top 10 selected inhibitors (n>25 for each group). Error bars, mean±SD. FIG. 5E, Counter screening using a mutant biosensor with a kinase-dead domain to subtract the noise engendered from non-specific fluorescence. The Scatter plot illustrates the FRET ratio changes in the positive and negative screenings using the saFRET biosensor fused with an active kinase or a kinase-dead domain, respectively. FIG. 5F: FRET ratio images of live-cell imaging with different inhibitors. The TAK-659 (10 μM) was used as the negative control, which cannot sufficiently inhibit the ZAP70 kinase. Scale bars, 10 μm. FIG. 5G: Time courses of the FRET ratio before and after inhibitor treatment (n>8 for each group). Error bars, mean±SEM.

### Verify the Inhibitor in Inhibiting Clinical-Relevant T Cell Activation

We tested the phosphorylation of LAT, a downstream substrate of ZAP70 kinase, and the activation of T cells with the top two identified inhibitors, staurosporine and AZD7762. Significant reductions of phosphorylated LAT (Y191) (FIG. 6A-C) and ZAP70 (FIG. 6D) were observed after treatments with staurosporine or AZD7762, verifying the accuracy of the biosensor screening results. These two small molecules also suppressed the expression of CD69 (FIG. 6E-F), a surface marker for activated T cells.

To examine the efficacy of staurosporine and AZD7762 in inhibiting pathological T cell activation, we used a disease model where T cell activation is mediated by ZAP70-R360P mutation, a main cause of a severe human autoimmune syndrome (Chan, Punwani et al. 2016). We introduced wild type ZAP70 or the R360P-mutant of ZAP70 into ZAP70-deficient P116 T cells (FIG. 6G). ZAP70-R360P led to enhanced T cell auto-activation compared to wild type ZAP70 (FIG. 6H). Staurosporine or AZD7762 significantly inhibited the phosphorylation of ZAP70 and LAT (Y191) (FIG. 6I-K), and the subsequently activation of T cells marked by CD69 (FIG. 6I). Hence, staurosporine or AZD7762 may be applicable to mitigate ZAP70R360P-mediated autoimmune disease. As such, the inhibitors identified through the saFRET-HTDS assay can efficiently inhibit the ZAP70 kinase signaling pathway and its subsequent T cell activation, which can have therapeutic potentials in treating diseases involving abnormal ZAP70 kinase or T cell activation.

In summary, we rationally designed the saFRET biosensor for drug screening and a novel and provide herein a systematic method to directly optimize saFRET biosensors in mammalian cells by a directed evolution approach. This method was proved to work well for Fyn- and Zap70-FRET biosensor optimization and can also be used for optimizing the sensitivity and specificity of FRET biosensors capable of monitoring tyrosine kinase signals crucial for the activation of immune cell.

FIG. 6: illustrates inhibition of T cell activation by the HTDS-identified ZAP70 inhibitors: Staurosporine and AZD7762. FIG. 6A, Experimental scheme and timeline for experiments in FIG. 6B-D. The Jurkat T cells were pre-treated with inhibitors for 30 minutes before anti-TCR stimulation by anti-CD3/CD28 antibodies for 5 minutes. FIG. 6B, Immunostaining images of pLAT (Y191) in Jurkat T cells with different inhibitor pre-treatments. Scale bars, 10 μm. FIG. 6D, Quantification of pLAT (Y191) intensity of single cells in different groups. (n>150 for each group, One-way ANOVA, ****P<0.0001). Error bars, mean±SD. FIG. 6D, Quantification of pZAP70 (Y493) intensity of single cells in different groups. (n>200 for each group, One-way ANOVA, ****P<0.0001). Error bars, mean±SD. FIG. 6E, Experimental scheme and timeline for CD69 staining experiment. FIG. 6F, Flow-cytometric analysis of CD69 expression in T cells after anti-TCR stimulation, with different inhibitor pre-treatments. FIG. 6G, Experimental scheme and timeline of P116 cells reconstituted with ZAP70. Full length ZAP70-WT or R360P were expressed with YPet via a cleavable P2A linker. P116 cells with similar ZAP70-WT or ZAP70-R360P expressions were sorted and isolated for further analysis based on YPet intensity. FIG. 6H, CD69 expression in P116 cells with or without the expression of ZAP70 (WT) and its mutant (R360P). FIG. 6I, Quantification of pZAP70 (Y493) intensity of single cells in different P116 groups. (n>100 for each group, One-way ANOVA, ****P<0.0001). Error bars, mean±SD. FIG. 6J, Images of pLAT (Y191) in P116-ZAP70 R360P cells with different inhibitor pre-treatments. Scale bars, 10 μm. FIG. 6K, Quantification of pLAT (Y191) intensity of single cells in P116-ZAP70 R360P cells with different inhibitor pre-treatment. (n>150 for each group, One-way ANOVA, ****P<0.0001). Error bars, mean±SD. 1, Flow-cytometric analysis of CD69 expression in P116-ZAP70-R360P cells with different inhibitor pre-treatment. ZAP70-WT or ZAP70-R360P expression levels were indicated by YPet intensity.

### Example 3: Making and Using Optimized ZAP70 Biosensors

We further applied the optimized ZAP70 biosensor to study how different CAR designs influence CAR-T cell functions. Since both ZAP70 and ERK kinases can be regulated by the CAR cytoplasmic tail and serve as key effectors for CAR signaling and T cell activation 19, 20 20, we examined the role of ITAM motif in regulating the ZAP70 and ERK kinases in response to CAR activation, utilizing our optimized ZAP70 biosensor and an ERK biosensor with high sensitivity 2. These FRET biosensors were co-expressed with the wild type CAR (19282, WT-CAR) or its mutated version (19282, XX3-CAR), which had inferior anti-tumor efficacy than its wild-type counterpart (the tyrosine sites of the first two ITAM motifs in the CAR cytoplasmic tail mutated to phenylalanines) 18. The kinase activity was tracked by live-cell imaging after the CAR-T cells expressing either WT-CAR or XX3-CAR were stimulated with antigen-presenting CD19⁺ 3T3 cells (FIG. 25A). A large FRET ratio change of ERK and ZAP70 biosensors in both types of CAR-T cells was observed when engaging with the CD19⁺ 3T3 cells; this indicates that ZAP70 and ERK kinases are both responsive to antigen stimulation (FIG. 25B-G). The ERK signals increased within 5 min after antigen engagement and quickly reached a plateau (FIG. 25B-C). However, no significant difference in ERK activity was observed between CAR-T cells expressing WT-CAR or XX3-CAR (FIG. 25D). In contrast, a significantly delayed response and a reduced activation level of ZAP70 kinase activity were observed in XX3-CAR T cells comparing to WT-CAR T cells (FIG. 25B-G), suggesting a significant ZAP70 defect in XX3-CAR-T cells. To verify the results of single-cell imaging, we further evaluated ZAP70 activity in cells expressing different CARs in a large-scale, high-throughput manner using flow cytometer with the optimized ZAP70 biosensor. Consistent with imaging experiments, a remarkable increase in the percentage of the ZAP70-active CAR T cells (as indicated by high-FRET ratios) was observed after CD19+ Raji stimulation (FIG. 25H-I). There was a significantly higher proportion of ZAP70-active cells in WT-CAR T cells than that of XX3-CAR T cells (FIG. 25J-L), confirming that WT-CAR is more effective in activating ZAP70 than XX3-CAR. These results suggest that ITAM motifs may affect ZAP70 to modulate CAR T cell functions. Our results also demonstrate that our optimized ZAP70 biosensor is applicable to monitoring and evaluating the signaling of different synthetic CAR molecules and enabling the high-throughput screening of functional/improved CARs by FACS.

We further verified that the FRET change induced by the identified inhibitors was specifically mediated by the change of the ZAP70 kinase domain. In fact, saFRET-HTDS enables the screening of inhibitors directly targeting ZAP70 Kinase with high specificity compared to conventional assays.

We have performed additional experiments to verify that our saFRET-HTDS platform can differentiate inhibitors of ZAP70 upstream molecules from those directly targeting ZAP70 itself, overcoming the drawback of the conventional FRET assays. We tested the inhibitors of signaling molecules acting upstream to ZAP70, for example, Src, Fyn, and Lck kinases, see FIG. 26A-D. Our results indicate that these non-specific inhibitors targeting kinases upstream to ZAP70 can be falsely selected by the conventional FRET assay), but not by the saFRET-HTDS assay, demonstrating the high specificity of our saFRET-HTDS screening approach.

### REFERENCES EXAMPLE 1

1. Zhang, J., et al. Creating new fluorescent probes for cell biology. Nature reviews Molecular cell biology 3, 906 (2002).
2. Komatsu, N. et al. Development of an optimized backbone of FRET biosensors for kinases and GTPases. Molecular biology of the cell 22, 4647-4656 (2011).
3. Nguyen, A.W. & Daugherty, P.S. Evolutionary optimization of fluorescent proteins for intracellular FRET. Nature biotechnology 23, 355-360 (2005).
4. Chan, A. Y. et al. A novel human autoimmune syndrome caused by combined hypomorphic and activating mutations in ZAP-70. J Exp Med 213, 155-165 (2016).
5. Hochreiter, B., Garcia, A.P. & Schmid, J.A. Fluorescent proteins as genetically encoded FRET biosensors in life sciences. Sensors (Basel) 15, 26281-26314 (2015).
6. Ibraheem, A., et al. A bacteria colony-based screen for optimal linker combinations in genetically encoded biosensors. BMC Biotechnology 11, 105 (2011).
7. Thestrup, T. et al. Optimized ratiometric calcium sensors for functional in vivo imaging of neurons and T lymphocytes. Nature Methods 11, 175 (2014).
8. Limsakul, P. et al. Directed Evolution to Engineer Monobody for FRET Biosensor Assembly and Imaging at Live-Cell Surface. Cell Chem Biol (2018).
9. Wang, P. Z. et al. Visualizing Spatiotemporal Dynamics of Intercellular Mechanotransmission upon Wounding. Acs Photonics 5, 3565-3574 (2018).
10. English, J. G. et al. VEGAS as a Platform for Facile Directed Evolution in Mammalian Cells. Cell 178, 748-761.e717 (2019).
11. Piatkevich, K. D. et al. A robotic multidimensional directed evolution approach applied to fluorescent voltage reporters. Nature Chemical Biology 14, 352-360 (2018).
12. Fritz, R. D. et al. A Versatile Toolkit to Produce Sensitive FRET Biosensors to Visualize Signaling in Time and Space. Science Signaling 6 (2013).
13. Um, J. W. et al. Alzheimer amyloid-β oligomer bound to postsynaptic prion protein activates Fyn to impair neurons. Nature Neuroscience 15, 1227-1235 (2012).
14. Weber, E. W. et al. Pharmacologic control of CAR-T cell function using dasatinib. Blood Advances 3, 711-717 (2019).
15. Au-Yeung, B. B., et al. ZAP-70 in Signaling, Biology, and Disease. Annual review of immunology 36, 127-156 (2018).
16. Rassenti, L. Z. et al. Relative value of ZAP-70, CD38, and immunoglobulin mutation status in predicting aggressive disease in chronic lymphocytic leukemia. Blood 112, 1923-1930 (2008).
17. Visperas, P. R. et al. Identification of Inhibitors of the Association of ZAP-70 with the T Cell Receptor by High-Throughput Screen. SLAS DISCOVERY: Advancing the Science of Drug Discovery 22, 324-331 (2016).
18. Wang, H. et al. ZAP-70: an essential kinase in T-cell signaling. Cold Spring Harb Perspect Biol 2, a002279 (2010).
19. Marine, S. et al. A miniaturized cell-based fluorescence resonance energy transfer assay for insulin-receptor activation. Analytical Biochemistry 355, 267-277 (2006).
20. Rothman, D. M., Shults, M. D. & Imperiali, B. Chemical approaches for investigating phosphorylation in signal transduction networks. Trends in Cell Biology 15, 502-510 (2005).
21. Lu, S. & Wang, Y. Fluorescence resonance energy transfer biosensors for cancer detection and evaluation of drug efficacy. Clin Cancer Res 16, 3822-3824 (2010).
22. Mizutani, T. et al. A novel FRET-based biosensor for the measurement of BCR-ABL activity and its response to drugs in living cells. Clin Cancer Res 16, 3964-3975 (2010).
23 Stroik, D. R. et al. Targeting protein-protein interactions for therapeutic discovery via FRET-based high-throughput screening in living cells. Scientific Reports 8, 12560 (2018).
24. Allen, M.D. et al. Reading Dynamic Kinase Activity in Living Cells for High-Throughput Screening. ACS Chemical Biology 1, 371-376 (2006).
25. Inglese, J. et al. High-throughput screening assays for the identification of chemical probes. Nature Chemical Biology 3, 466-479 (2007).
26. Lun, X.-K. & Bodenmiller, B. Profiling Cell Signaling Networks at Single-cell Resolution. Mol Cell Proteomics 19, 744-756 (2020).
27. Ouyang, M. et al. A sensitive FRET biosensor reveals Fyn kinase regulation by sub-membrane localization. ACS sensors (2018).
28. Songyang, Z. et al. Catalytic Specificity of Protein-Tyrosine Kinases Is Critical for Selective Signaling. Nature 373, 536-539 (1995).
29 Nair, S. A. et al. Identification of efficient pentapeptide substrates for the tyrosine kinase pp60c-src. Journal of medicinal chemistry 38, 4276-4283 (1995).
30. Songyang, Z. & Cantley, L.C. SH2 domain specificity determination using oriented phosphopeptide library. Methods in enzymology 254, 523-535 (1995).
31. Zheng, L., Baumann, U. & Reymond, J.-L. An efficient one-step site-directed and site-saturation mutagenesis protocol. Nucleic acids research 32, e115-e115 (2004).
32 Twamley-Stein, G. M., et al. The Src family tyrosine kinases are required for platelet-derived growth factor-mediated signal transduction in NIH 3T3 cells. Proceedings of the National Academy of Sciences 90, 7696-7700 (1993).
33. Fowler, D.M. & Fields, S. Deep mutational scanning: a new style of protein science. Nature methods 11, 801 (2014).
34 Fowler, D. M. et al. High-resolution mapping of protein sequence-function relationships. Nature methods 7, 741 (2010).
35. Ouyang, M. et al. Simultaneous visualization of protumorigenic Src and MT1-MMP activities with fluorescence resonance energy transfer. Cancer Res 70, 2204-2212 (2010).
36. Li, K. et al. Imaging Spatiotemporal Activities of ZAP-70 in Live T Cells Using a FRET-Based Biosensor. Ann Biomed Eng 44, 3510-3521 (2016).
37 Randriamampita, C. et al. A Novel ZAP-70 Dependent FRET Based Biosensor Reveals Kinase Activity at both the Immunological Synapse and the Antisynapse. PLOS ONE 3, e1521 (2008).
38. Cadra, S. et al. ROZA-XL, an improved FRET based biosensor with an increased dynamic range for visualizing zeta associated protein 70 kD (ZAP-70) tyrosine kinase activity in live T cells. Biochem Biophys Res Commun 459, 405-410 (2015).
39. Lam, B. et al. Discovery of TAK-659 an orally available investigational inhibitor of Spleen Tyrosine Kinase (SYK). Bioorganic & Medicinal Chemistry Letters 26, 5947-5950 (2016).
40. Brandvold, K. R., et al. Development of a highly selective c-Src kinase inhibitor. ACS chemical biology 7, 1393-1398 (2012).
41. Huby, R. D., et al. ZAP-70 protein tyrosine kinase is constitutively targeted to the T cell cortex independently of its SH2 domains. J Cell Biol 137, 1639-1649 (1997).
42. Wan, R. et al. Biophysical basis underlying dynamic Lck activation visualized by ZapLck FRET biosensor. Science Advances 5, eaau2001 (2019).
43. Filipp, D., Ballek, O. & Manning, J. Lck, Membrane Microdomains, and TCR Triggering Machinery: Defining the New Rules of Engagement. Frontiers in Immunology 3 (2012).
44. Kabouridis, P.S. Lipid rafts in T cell receptor signalling (Review). Molecular Membrane Biology 23, 49-57 (2006).
45. Kovacs, B. et al. Human CD8+ T cells do not require the polarization of lipid rafts for activation and proliferation. Proceedings of the National Academy of Sciences 99, 15006-15011 (2002).
46. Seong, J. et al. Visualization of Src activity at different compartments of the plasma membrane by FRET imaging. Chemistry & biology 16, 48-57 (2009).
47 Lo, W.-L. et al. Lck promotes Zap70-dependent LAT phosphorylation by bridging Zap70 to LAT. Nature immunology 19, 733-741 (2018).
48. Simeonov, A. & Davis, M. I. in Assay Guidance Manual. (eds. S. Markossian et al.) (Eli Lilly & Company and the National Center for Advancing Translational Sciences, Bethesda (MD); 2004).
49. Wu, Z., Kan, S. B. J., et al. Proceedings of the National Academy of Sciences 116, 8852-8858 (2019).
50. Shah, N.H. et al. An electrostatic selection mechanism controls sequential kinase signaling downstream of the T cell receptor. eLife 5, e20105 (2016).
51. Lo, W.-L. et al. Nature Immunology 20, 1481-1493 (2019).
52. Moffat, J. G., Rudolph, J. & Bailey, D. Phenotypic screening in cancer drug discovery-past, present and future. Nature reviews. Drug discovery 13, 588-602 (2014).
53. Zhao, H. et al., Lab on a Chip 15, 3481-3494 (2015).
54. Wade, M., et al. in Assay Guidance Manual. (eds. G.S. Sittampalam et al.) (Bethesda (MD); 2004).
55. Mócsai, A., et al. The SYK tyrosine kinase: a crucial player in diverse biological functions. Nature Reviews Immunology 10, 387-402 (2010).
56. MacGlashan Jr., D. Stability of Syk protein and mRNA in human peripheral blood basophils. Journal of Leukocyte Biology 100, 535-543 (2016).
57. Heynen-Genel, S., et al. Functional genomic and high-content screening for target discovery and deconvolution. Expert Opin Drug Discov 7, 955-968 (2012).

A number of embodiments of the invention have been described. Nevertheless, it can be understood that various modifications may be made without departing from the spirit and scope of the invention. Accordingly, other embodiments are within the scope of the following claims.

1: A chimeric, synthetic polypeptide comprising:
a first chimeric peptide module comprising an enhanced cyan fluorescent protein (CFP) (ECFP) domain amino terminal to a Src Homology 2 (SH2) domain or equivalent;
a second chimeric peptide module, attached to the carboxy-terminal of the SH2 domain or equivalent of the first peptide module by a peptide linker, comprising: a kinase substrate domain capable of being phosphorylate by the kinase; and, a fluorescent protein domain ;
a third chimeric peptide module, attached to the carboxy-terminal of the YPet domain of the second peptide module by a peptide linker, comprising a polypeptide having a kinase activity,
wherein the chimeric polypeptide acts as a self-activating Förster resonance energy transfer (saFRET) biosensor,
and the chimeric peptide has a general structure:
ECFP-SH2-linker-substrate-YPet-Kinase domain.

2: The chimeric, synthetic polypeptide of claim 1, wherein the polypeptide having a kinase activity is a tyrosine kinase, or a Fyn or a ZAP70 kinase.

3: A nucleic acid encoding the chimeric, synthetic polypeptide of claim 1.

4: An expression vector comprising or having contained therein a nucleic acid of claim 3.

5: A cell comprising or having contained therein a nucleic acid of claim 3.

6: A method for identifying a kinase inhibitor in a cell, comprising:
(a) providing a cell expressing a synthetic polypeptide encoded by a nucleic acid of claim 3,
(b) providing a test molecule;
(c) contacting the test molecule with the cell and measuring or detecting a change in a detectable signal generated by the synthetic polypeptide, as compared to a cell expressing the synthetic polypeptide , that has not been contacted with the test molecule, wherein a change in detectable signal identifies the test molecule as an inhibitor of the polypeptide having the kinase activity in the third chimeric peptide module.

7: The chimeric, synthetic polypeptide of claim 1, wherein the second chimeric peptide module, attached to the carboxy-terminal of the SH2 domain or equivalent of the first peptide module by a flexible peptide linker.

8: The chimeric, synthetic polypeptide of claim 1, wherein the fluorescent protein domain comprises a basic, constitutively fluorescent, yellow fluorescent protein-comprising domain, or a YPet domain; or equivalents.

9: The chimeric, synthetic polypeptide of claim 1, wherein the third chimeric peptide module is attached to the carboxy-terminal of the YPet domain of the second peptide module by a flexible peptide linker.

10: The cell of claim 5, wherein the cell comprises or has contained therein a chimeric polypeptide of claim 1.

11: The cell of claim 5, wherein cell is a human cell.

12: The cell of claim 5, wherein the cell is a lymphocyte or a T cell, or a CAR T cell.

13: The cell of claim 6, wherein the cell is a human cell, or a human lymphocyte or human T cell, or a human CAR T cell.

14: The method of claim 6, wherein the cell is a human cell.

15: The method of claim 6, wherein the cell is a lymphocyte or a T cell, or a CAR T cell.

16: The method of claim 6, wherein the cell is a human cell, or a human lymphocyte or human T cell, or a human CAR T cell.

17: The method of claim 6, wherein the test molecule is a synthetic molecule or a molecule from a kinase inhibitor library.
